SEQUENCE LISTING (75 sequences)

SEQ ID NO: 1 - protein, 5 aa, synthetic construct
  REGION 1..5: note = VH CDR1 of anti-CLL1 antibody
GYYMH

SEQ ID NO: 2 - protein, 5 aa, synthetic construct
  REGION 1..5: note = VH CDR1 of anti-CLL1 antibody
GYAMN

SEQ ID NO: 3 - protein, 5 aa, synthetic construct
  REGION 1..5: note = VH CDR1 of anti-CLL1 antibody
SYVIH

SEQ ID NO: 4 - protein, 17 aa, synthetic construct
  REGION 1..17: note = VH CDR2 of anti-CLL1 antibody
HVNPYNGATSFNRNFKD

SEQ ID NO: 5 - protein, 17 aa, synthetic construct
  REGION 1..17: note = VH CDR2 of anti-CLL1 antibody
LINPYNGGAMYNQKFKG

SEQ ID NO: 6 - protein, 17 aa, synthetic construct
  REGION 1..17: note = VH CDR2 of anti-CLL1 antibody
LFNPYNDDVNYNEKFKG

SEQ ID NO: 7 - protein, 12 aa, synthetic construct
  REGION 1..12: note = VH CDR3 of anti-CLL1 antibody
SADGYYERHFDY

SEQ ID NO: 8 - protein, 10 aa, synthetic construct
  REGION 1..10: note = VH CDR3 of anti-CLL1 antibody
DYRYDGHLDY

SEQ ID NO: 9 - protein, 12 aa, synthetic construct
  REGION 1..12: note = VH CDR3 of anti-CLL1 antibody
EGVHYGRPWFGY

SEQ ID NO: 10 - protein, 12 aa, synthetic construct
  REGION 1..12: note = VH CDR3 of anti-CLL1 antibody
SAYGYYERHFDY

SEQ ID NO: 11 - protein, 10 aa, synthetic construct
  REGION 1..10: note = VH CDR3 of anti-CLL1 antibody
DYRYEGHLDY

SEQ ID NO: 12 - protein, 15 aa, synthetic construct
  REGION 1..15: note = VL CDR1 of anti-CLL1 antibody
KASQSVDYDGDSYMN

SEQ ID NO: 13 - protein, 10 aa, synthetic construct
  REGION 1..10: note = VL CDR1 of anti-CLL1 antibody
SATSSVSYMH

SEQ ID NO: 14 - protein, 11 aa, synthetic construct
  REGION 1..11: note = VL CDR1 of anti-CLL1 antibody
KASQDIKSYLN

SEQ ID NO: 15 - protein, 15 aa, synthetic construct
  REGION 1..15: note = VL CDR1 of anti-CLL1 antibody
KASQSVDYDADSYMN

SEQ ID NO: 16 - protein, 7 aa, synthetic construct
  REGION 1..7: note = VL CDR2 of anti-CLL1 antibody
AASNLQS

SEQ ID NO: 17 - protein, 7 aa, synthetic construct
  REGION 1..7: note = VL CDR2 of anti-CLL1 antibody
DTSKLAS

SEQ ID NO: 18 - protein, 7 aa, synthetic construct
  REGION 1..7: note = VL CDR2 of anti-CLL1 antibody
RASRLVD

SEQ ID NO: 19 - protein, 9 aa, synthetic construct
  REGION 1..9: note = VL CDR3 of anti-CLL1 antibody
QQSDKDPLT

SEQ ID NO: 20 - protein, 9 aa, synthetic construct
  REGION 1..9: note = VL CDR3 of anti-CLL1 antibody
QQWSSDSPT

SEQ ID NO: 21 - protein, 9 aa, synthetic construct
  REGION 1..9: note = VL CDR3 of anti-CLL1 antibody
LQYDEFPLT

SEQ ID NO: 22 - protein, 9 aa, synthetic construct
  REGION 1..9: note = VL CDR3 of anti-CLL1 antibody
QQSDRDPLT

SEQ ID NO: 23 - protein, 330 aa, synthetic construct
  REGION 1..330: note = Heavy chain constant region of anti-CLL1 antibody
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 24 - protein, 330 aa, synthetic construct
  REGION 1..330: note = Heavy chain constant region N297A mutant of anti-CLL1 antibody
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 25 - protein, 121 aa, synthetic construct
  REGION 1..121: note = Heavy chain variable region of anti-CLL1 antibody
EVQLQQSGPELVKPGASVKISCKASGYSFTGYYMHWVKQSHVKSLEWIGHVNPYNGATSFNRNFKDKASLTVDKSSSTAYMELHSLTSEDSAVYYCGRSADGYYERHFDYWGQGTTLTVSS

SEQ ID NO: 26 - protein, 119 aa, synthetic construct
  REGION 1..119: note = Heavy chain variable region of anti-CLL1 antibody
EVQLQQSGPELVKPGASMKISCKASGYSFTGYAMNWVKQSHGKNLEWIGLINPYNGGAMYNQKFKGKATLTVDKSTSTAYMELLSLTSEDSAVYYCARDYRYDGHLDYWGQGTTLTVSS

SEQ ID NO: 27 - protein, 121 aa, synthetic construct
  REGION 1..121: note = Heavy chain variable region of anti-CLL1 antibody
EVQLQQSGPELVKSGASVKMSCKASGYTFTSYVIHWVKQMPGQGLEWIGLFNPYNDDVNYNEKFKGKATLTSDKYSSTAYLDLSSLTSEDSAVYYCAREGVHYGRPWFGYWGQGTLVTVSA

SEQ ID NO: 28 - protein, 121 aa, synthetic construct
  REGION 1..121: note = Heavy chain variable region of anti-CLL1 antibody
EVQLQQSGPELVKPGASVKISCKASGYSFTGYYMHWVKQSHVKSLEWIGHVNPYNGATSFNRNFKDKASLTVDKSSSTAYMELHSLTSEDSAVYYCGRSAYGYYERHFDYWGQGTTLTVSS

SEQ ID NO: 29 - protein, 119 aa, synthetic construct
  REGION 1..119: note = Heavy chain variable region of anti-CLL1 antibody
EVQLQQSGPELVKPGASMKISCKASGYSFTGYAMNWVKQSHGKNLEWIGLINPYNGGAMYNQKFKGKATLTVDKSTSTAYMELLSLTSEDSAVYYCARDYRYEGHLDYWGQGTTLTVSS

SEQ ID NO: 30 - protein, 121 aa, synthetic construct
  REGION 1..121: note = Heavy chain variable region of anti-CLL1 antibody
QVQLVQSGAEVKKPGASVKVSCKASGYSFTGYYMHWVRQAPGQGLEWMGHVNPYNGATSFNRNFKDRVTMTRDTSTSTVYMELSSLRSEDTAVYYCARSADGYYERHFDYWGQGTLVTVSS

SEQ ID NO: 31 - protein, 121 aa, synthetic construct
  REGION 1..121: note = Heavy chain variable region of anti-CLL1 antibody
QVQLVQSGAEVKKPGASVKVSCKASGYSFTGYYMHWVRQAPGQGLEWIGHVNPYNGATSFNRNFKDRVTMTVDTSTSTVYMELSSLRSEDTAVYYCGRSADGYYERHFDYWGQGTLVTVSS

SEQ ID NO: 32 - protein, 121 aa, synthetic construct
  REGION 1..121: note = Heavy chain variable region of anti-CLL1 antibody
QVQLVQSGAEVKKPGASVKISCKASGYSFTGYYMHWVKQSHGQGLEWIGHVNPYNGATSFNRNFKDRASLTVDTSTSTVYMELSSLRSEDSAVYYCGRSADGYYERHFDYWGQGTLVTVSS

SEQ ID NO: 33 - protein, 119 aa, synthetic construct
  REGION 1..119: note = Heavy chain variable region of anti-CLL1 antibody
QVQLVQSGAEVKKPGASVKVSCKASGYSFTGYAMNWVRQAPGQRLEWIGLINPYNGGAMYNQKFKGRATLTVDTSASTAYMELSSLRSEDTAVYYCARDYRYDGHLDYWGQGTLVTVSS

SEQ ID NO: 34 - protein, 119 aa, synthetic construct
  REGION 1..119: note = Heavy chain variable region of anti-CLL1 antibody
QVQLVQSGAEVKKPGASVKVSCKASGYSFTGYAMNWVRQAPGQRLEWIGLINPYNGGAMYNQKFKGRATLTVDTSASTAYMELSSLRSEDTAVYYCARDYRYEGHLDYWGQGTLVTVSS

SEQ ID NO: 35 - protein, 119 aa, synthetic construct
  REGION 1..119: note = Heavy chain variable region of anti-CLL1 antibody
QVQLVQSGAEVKKPGASVKISCKASGYSFTGYAMNWVKQAPGQRLEWIGLINPYNGGAMYNQKFKGRATLTVDTSASTAYMELSSLRSEDTAVYYCARDYRYDGHLDYWGQGTLVTVSS

SEQ ID NO: 36 - protein, 111 aa, synthetic construct
  REGION 1..111: note = Light chain variable region of anti-CLL1 antibody
DIVLTQSPASLAVSLGQRATISCKASQSVDYDGDSYMNWYQQKPGQPPKLLIYAASNLQSGIPARFSGSGSGTDFTLNIHPVEEEDAATYYCQQSDKDPLTFGAGTKLELK

SEQ ID NO: 37 - protein, 106 aa, synthetic construct
  REGION 1..106: note = Light chain variable region of anti-CLL1 antibody
QIVLTQSPVIMSASPGEKVTMTCSATSSVSYMHWYQQKSGTSPKRWIYDTSKLASGVPVRFSGSGSGTSYSLTISRMEAEDAATYYCQQWSSDSPTFGVGTKLELK

SEQ ID NO: 38 - protein, 107 aa, synthetic construct
  REGION 1..107: note = Light chain variable region of anti-CLL1 antibody
DIKMTQSPSSMNASLGERVTITCKASQDIKSYLNWFQQKPGKSPKTLIYRASRLVDGVPARFSGSESGQDYSLTISSLEFEDMGIYYCLQYDEFPLTFGAGTKLELK

SEQ ID NO: 39 - protein, 111 aa, synthetic construct
  REGION 1..111: note = Light chain variable region of anti-CLL1 antibody
DIVLTQSPASLAVSLGQRATISCKASQSVDYDADSYMNWYQQKPGQPPKLLIYAASNLQSGIPARFSGSGSGTDFTLNIHPVEEEDAATYYCQQSDRDPLTFGAGTKLELK

SEQ ID NO: 40 - protein, 107 aa, synthetic construct
  REGION 1..107: note = Light chain variable region N12S mutant of anti-CLL1 antibody
DIKMTQSPSSMSASLGERVTITCKASQDIKSYLNWFQQKPGKSPKTLIYRASRLVDGVPARFSGSESGQDYSLTISSLEFEDMGIYYCLQYDEFPLTFGAGTKLELK

SEQ ID NO: 41 - protein, 111 aa, synthetic construct
  REGION 1..111: note = Light chain variable region of anti-CLL1 antibody
DIVMTQSPDSLAVSLGERATINCKASQSVDYDGDSYMNWYQQKPGQPPKLLIYAASNLQSGVPDRFSGSGSGTDFTLTISSLQAEDVAVYYCQQSDKDPLTFGGGTKLEIK

SEQ ID NO: 42 - protein, 106 aa, synthetic construct
  REGION 1..106: note = Light chain variable region of anti-CLL1 antibody
EIVLTQSPATLSLSPGERATLSCSATSSVSYMHWYQQKPGQAPRRWIYDTSKLASGIPARFSGSGSGTSYTLTISSLEPEDFAVYYCQQWSSDSPTFGQGTKLEIK

SEQ ID NO: 43 - protein, 106 aa, synthetic construct
  REGION 1..106: note = Light chain variable region of anti-CLL1 antibody
EIVLTQSPATLSLSPGERATMSCSATSSVSYMHWYQQKPGTAPRRWIYDTSKLASGIPARFSGSGSGTDYTLTISSLEPEDFAVYYCQQWSSDSPTFGQGTKLEIK

SEQ ID NO: 44 - protein, 106 aa, synthetic construct
  REGION 1..106: note = Light chain variable region of anti-CLL1 antibody
EIVLTQSPATLSLSPGERATLSCSATSSVSYMHWYQQKSGTAPRRWIYDTSKLASGIPARFSGSGSGTDYTLTISSLEPEDAAVYYCQQWSSDSPTFGQGTKLEIK

SEQ ID NO: 45 - protein, 222 aa, synthetic construct
  REGION 1..222: note = Heavy chain of anti-CLL1 antibody
QVQLVQSGAEVKKPGASVKVSCKASGYSFTGYAMNWVRQAPGQRLEWIGLINPYNGGAMYNQKFKGRATLTVDTSASTAYMELSSLRSEDTAVYYCARDYRYEGHLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 46 - protein, 213 aa, synthetic construct
  REGION 1..213: note = Light chain of anti-CLL1 antibody
EIVLTQSPATLSLSPGERATLSCSATSSVSYMHWYQQKPGQAPRRWIYDTSKLASGIPARFSGSGSGTSYTLTISSLEPEDFAVYYCQQWSSDSPTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 47 - protein, 10 aa, synthetic construct
  REGION 1..10: note = VH CDR1 of anti-CD3 antibody
GFAFTDYYIH

SEQ ID NO: 48 - protein, 17 aa, synthetic construct
  REGION 1..17: note = VH CDR2 of anti-CD3 antibody
WISPGNVNTKYNENFKG

SEQ ID NO: 49 - protein, 10 aa, synthetic construct
  REGION 1..10: note = VH CDR3 of anti-CD3 antibody
DGYSLYYFDY

SEQ ID NO: 50 - protein, 17 aa, synthetic construct
  REGION 1..17: note = VL CDR1 of anti-CD3 antibody
KSSQSLLNSRTRKNYLA

SEQ ID NO: 51 - protein, 7 aa, synthetic construct
  REGION 1..7: note = VL CDR2 of anti-CD3 antibody
WASTRQS

SEQ ID NO: 52 - protein, 8 aa, synthetic construct
  REGION 1..8: note = VL CDR3 of anti-CD3 antibody
TQSHTLRT

SEQ ID NO: 53 - protein, 246 aa, synthetic construct
  REGION 1..246: note = Heavy chain of anti-CD3 antibody
QVQLVQSGAEVKKPGSSVKVSCKASGFAFTDYYIHWVRQAPGQGLEWMGWISPGNVNTKYNENFKGRVTITADKSTSTAYMELSSLRSEDTAVYYCARDGYSLYYFDYWGQGTLVTVLEDLKNVFPPEVAVFEPSEAEISHTQKATLVCLATGFYPDHVELSWWVNGKEVHSGVCTDPQPLKEQPALQDSRYALSSRLRVSATFWQNPRNHFRCQVQFYGLSENDEWTQDRAKPVTQIVSAEAWGR

SEQ ID NO: 54 - protein, 207 aa, synthetic construct
  REGION 1..207: note = Light chain of anti-CD3 antibody
DIVMTQSPDSLAVSLGERATINCKSSQSLLNSRTRKNYLAWYQQKPGQPPKLLIYWASTRQSGVPDRFSGSGSGTDFTLTISSLQAEDVAVYYCTQSHTLRTFGGGTKVEIKPDIQNPDPAVYQLRDSKSSDKSVCLFTDFDSQTQVSQSKDSDVYITDKCVLDMRSMDFKSNSAVAWSQKSDFACANAFQNSIIPEDTFFPSPESS

SEQ ID NO: 55 - protein, 107 aa, synthetic construct [sequence omitted]

SEQ ID NO: 56 - protein, 229 aa, synthetic construct
  REGION 1..229: note = Fc of anti-CLL1/anti-CD3 bispecific antibody
ASDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 57 - protein, 227 aa, synthetic construct
  REGION 1..227: note = Fc of anti-CLL1/anti-CD3 bispecific antibody
DKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 58 - protein, 5 aa, synthetic construct
  REGION 1..5: note = peptide linker
GGGGS

SEQ ID NO: 59 - protein, 10 aa, synthetic construct
  REGION 1..10: note = peptide linker
GGGGSGGGGS

SEQ ID NO: 60 - protein, 10 aa, synthetic construct
  REGION 1..10: note = peptide linker
SGGGGSGGGG

SEQ ID NO: 61 - protein, 14 aa, synthetic construct
  REGION 1..14: note = peptide linker
GGGGSGGGGSGGGG

SEQ ID NO: 62 - protein, 10 aa, synthetic construct
  REGION 1..10: note = peptide linker
GSPGSSSSGS

SEQ ID NO: 63 - protein, 15 aa, synthetic construct
  REGION 1..15: note = peptide linker
GGGGSGGGGSGGGGS

SEQ ID NO: 64 - protein, 8 aa, synthetic construct
  REGION 1..8: note = peptide linker
GSGSGSGS

SEQ ID NO: 65 - protein, 8 aa, synthetic construct
  REGION 1..8: note = peptide linker
GSGSGNGS

SEQ ID NO: 66 - protein, 8 aa, synthetic construct
  REGION 1..8: note = peptide linker
GGSGSGSG

SEQ ID NO: 67 - protein, 6 aa, synthetic construct
  REGION 1..6: note = peptide linker
GGSGSG

SEQ ID NO: 68 - protein, 4 aa, synthetic construct
  REGION 1..4: note = peptide linker
GGSG

SEQ ID NO: 69 - protein, 8 aa, synthetic construct
  REGION 1..8: note = peptide linker
GGSGNGSG

SEQ ID NO: 70 - protein, 8 aa, synthetic construct
  REGION 1..8: note = peptide linker
GGNGSGSG

SEQ ID NO: 71 - protein, 6 aa, synthetic construct
  REGION 1..6: note = peptide linker
GGNGSG

SEQ ID NO: 72 - protein, 468 aa, synthetic construct
  REGION 1..468: note = hFc-human CLL1
MYRMQLLSCIALSLALVTNSAPLEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGARTGGGGSGGGGSHVTLKIEMKKMNKLQNISEELQRNISLQLMSNMNISNKIRNLSTTLQTIATKLCRELYSKEQEHKCKPCPRRWIWHKDSCYFLSDDVQTWQESKMACAAQNASLLKINNKNALEFIKSQSRSYDYWLGLSPEEDSTRGMRVDNIINSSAWVIRNAPDLNNMYCGYINRLYVQYYHCTYKKRMICEKMANPVQLGSTYFREA

SEQ ID NO: 73 - protein, 468 aa, synthetic construct
  REGION 1..468: note = hFc-cynomolgus CLL1
MYRMQLLSCIALSLALVTNSAPLEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGARTGGGGSGGGGSHITLKTAMKKMNKLQNINEELQRNVSLQLMSNMNSSNKIRNLSTTLQTIATRLCRELYSKEQEHKCKPCPRRWIWHKDSCYFLSDDVRTWQESRMACAAQNASLLKINNKNALEFIKSQSTSYPYWLGLSPEKDYSYGTSVDDIINSSAWVTRNASDLNNMFCGYINRIYVHYDYCIYRKKMICEKMANPVQLGFIHFREA

SEQ ID NO: 74 - protein, 121 aa, synthetic construct
  REGION 1..121: note = Heavy chain variable region of anti-CLL1 antibody
QVQLVQSGAEVKKPGASVKISCKASGYSFTGYYMHWVKQSHGQGLEWIGHVNPYNGATSFNRNFKDRASLTVDTSTSTVYMELSSLRSEDSAVYYCGRSAYGYYERHFDYWGQGTLVTVSS

SEQ ID NO: 75 - protein, 111 aa, synthetic construct
  REGION 1..111: note = Light chain variable region of anti-CLL1 antibody
DIVMTQSPDSLAVSLGERATINCKASQSVDYDADSYMNWYQQKPGQPPKLLIYAASNLQSGVPDRFSGSGSGTDFTLTISSLQAEDVAVYYCQQSDRDPLTFGGGTKLEIK

(19) United States
(12) Patent Application Publication
(10) Pub. No.: US 2025/0084158 A1
(43) Pub. Date: Mar. 13, 2025

(54) ANTI-CLL-1 ANTIBODIES AND USES THEREOF

(71) Applicant: ABL BIO INC., Seongnam-si Gyeonggi-do (KR)

(72) Inventors: Eunsil SUNG, Seongnam-si Gyeonggi-do (KR); Junga KWON, Seongnam-si Gyeonggi-do (KR); Youngkwang KIM, Seongnam-si Gyeonggi-do (KR); Juhee KIM, Seongnam-si Gyeonggi-do (KR); Kyungjin PARK, Seongnam-si Gyeonggi-do (KR); Sumyeong PARK, Seongnam-si Gyeonggi-do (KR); Byungje SUNG, Seongnam-si Gyeonggi-do (KR); Byeongmin YOO, Seongnam-si Gyeonggi-do (KR); Bora LEE, Seongnam-si Gyeonggi-do (KR); Suyoun LEE, Seongnam-si Gyeonggi-do (KR); Shinai LEE, Seongnam-si Gyeonggi-do (KR); Yangsoon LEE, Seongnam-si Gyeonggi-do (KR); Eunhee LEE, Seongnam-si Gyeonggi-do (KR); Yangmi LIM, Seongnam-si Gyeonggi-do (KR); Jaehyoung JEON, Seongnam-si Gyeonggi-do (KR); Jinwon JUNG, Seongnam-si Gyeonggi-do (KR)

(73) Assignee: ABL BIO INC., Seongnam-si Gyeonggi-do (KR)

(21) Appl. No.: 18/580,552

(22) PCT Filed: Jul. 20, 2022
(86) PCT No.: PCT/IB2022/056676

§ 371 Date: Jan. 18, 2024

(30) Foreign Application Priority Data
Jul. 20, 2021 (KR) 10-2021-0095142
Dec. 10, 2021 (KR) 10-2021-0176638

(51) Int. Cl. C07K 16/24; A61K 39/00; A61P 35/00; C07K 16/28

(52) U.S. Cl. CPC C07K 16/24; A61P 35/00; C07K 16/2809; A61K 2039/505; C07K 2317/24; C07K 2317/31; C07K 2317/33; C07K 2317/732; C07K 2317/92

(57) ABSTRACT

Provided are anti-CLL-1 antibodies and uses thereof, according to the anti-CLL-1 antibody of one aspect, it can bind to CLL-1 with high binding affinity, and can cause activation of T cells, thus it can be used effectively for preventing or treating the cancer expressing CLL-1.

### CROSS-REFERENCE TO RELATED APPLICATION

This application is based on and claims priority to Korean Provisional Patent Application No. 10-2021-0095142, filed on Jul. 20, 2021, and Korean Provisional Patent Application No. 10-2021-0176638, filed on Dec. 10, 2021, in the Korean Intellectual Property Office, the disclosure of which is incorporated by reference herein in its entirety.

Provided are anti-CLL-1 antibodies and uses thereof.

### TECHNICAL FIELD

### Background Art

Acute Myeloid leukemia (AML) is the most common and fatal hematological malignancy in adult patients where the majority have a poor prognosis. AML remains difficult to treat due to the heterogeneity of the disease, lack of specific target antigens, and presence of leukemic stem cells (LSCs) that mediate relapse after conventional treatments.

CLEC12A (C-type lectin domain family 12 member A; also known as C-type lectin-like molecule-1 [CLL-1], CD371, dendritic cell-associated lectin 2 [DCAL2], myeloid inhibitory C-type lectin-like receptor [MICL] and killer cell lectin like receptor-1 [KLRL1]) is a myeloid differentiation antigen expressed on ˜90% of newly diagnosed and relapsed AML. It is a type II transmembrane glycoprotein comprising an extracellular C-terminal lectin domain, transmembrane region, and the N-terminal cytoplasmic tail.

Monoclonal antibody (mAb)-based therapy has become an important treatment modality for cancer. Leukemia is well suited to this approach because of the accessibility of malignant cells in the blood, bone marrow, spleen, and lymph nodes and the well-defined immunophenotypes of the various lineages and stages of hematopoietic differentiation that permit identification of antigenic targets. Most studies for acute myeloid leukemia (AML) have focused on CD33. However, responses with the unconjugated anti-CD33 mAb lintuzumab have had modest single agent and activity against AML and failed to improve patient outcomes in two randomized trials when combined with conventional chemotherapy.

There is a need in the art for safe and effective agents that target AML including CLL-1 for the diagnosis and treatment of CLL-1-associated conditions, such as cancer. The invention fulfills that need and provides other benefits.

### DISCLOSURE

### Technical Problem

An aspect of the present disclosure provides an isolated anti-CLL-1 or an antigen-binding fragment thereof.

Another aspect of the present disclosure provides an isolated nucleic acid encoding the anti-CLL-1 antibody.

Another aspect of the present disclosure provides a vector comprising the isolated nucleic acid.

Another aspect of the present disclosure provides a host cell comprising the vector.

Another aspect of the present disclosure provides a pharmaceutical composition comprising the anti-CLL-1 antibody.

Another aspect of the present disclosure provides a method for treating or preventing a cancer in a patient in need thereof, comprising administering to the patient an effective amount of the anti-CLL-1 antibody.

Another aspect of the present disclosure provides a use of the anti-CLL-1 antibody in the manufacture of medicament for treating or preventing a cancer.

Another aspect of the present disclosure provides a use of the anti-CLL-1 antibody for treating or preventing a cancer.

### Technical Solution

An aspect of the present disclosure provides an isolated anti-CLL-1 antibody or an antigen-binding fragment thereof comprising: (a) a VH CDR1 comprising an amino acid sequence selected from the group consisting of SEQ ID NOs: 1, 2 and 3; (b) a VH CDR2 comprising an amino acid sequence selected from the group consisting of SEQ ID NOs: 4, 5 and 6; (c) a VH CDR3 comprising an amino acid sequence selected from the group consisting of SEQ ID NOs: 7, 8, 9, 10 and 11; (d) a VL CDR1 comprising an amino acid sequence selected from the group consisting of SEQ ID NOs: 12, 13, 14 and 15; (e) a VL CDR2 comprising an amino acid sequence selected from the group consisting of SEQ ID NOs: 16, 17 and 18; and (f) a VL CDR3 comprising an amino acid sequence selected from the group consisting of SEQ ID NOs: 19, 20, 21 and 22.

In an embodiment, the anti-CLL-1 antibody or fragment thereof may comprise a heavy chain constant region comprising an amino acid sequence selected from the group consisting of SEQ ID NOs: 23 and 24.

In an embodiment, the anti-CLL-1 antibody or fragment thereof may comprise a light chain constant region comprising an amino acid sequence consisting of SEQ ID NO: 55.

In an embodiment, the anti-CLL-1 antibody or fragment thereof may comprise a heavy chain constant region comprising an amino acid sequence selected from the group consisting of SEQ ID NOs: 23 and 24; and a light chain constant region comprising an amino acid sequence consisting of SEQ ID NO: 55.

In an embodiment, the anti-CLL-1 antibody or fragment thereof may comprise a heavy chain variable region comprising an amino acid sequence selected from the group consisting of SEQ ID NOs: 25, 26, 27, 28, 29, 30, 31, 32, 33, 34, 35 and 74.

In an embodiment, the anti-CLL-1 antibody or fragment thereof may comprise a light chain variable region comprising an amino acid sequence selected from the group consisting of SEQ ID NOs: 36, 37, 38, 39, 40, 41, 42, 43, 44 and 75.

In an embodiment, the anti-CLL-1 antibody or fragment thereof may comprise a heavy chain variable region comprising an amino acid sequence selected from the group consisting of SEQ ID NOs: 25, 26, 27, 28, 29, 30, 31, 32, 33, 34, 35 and 74; and a light chain variable region comprising an amino acid sequence selected from the group consisting of SEQ ID NOs: 36, 37, 38, 39, 40, 41, 42, 43, 44 and 75.

In an embodiment, the anti-CLL-1 antibody or fragment thereof may comprise a heavy chain comprising an amino acid sequence consisting of SEQ ID NO: 45.

In an embodiment, the anti-CLL-1 antibody or fragment thereof may comprise a light chain comprising an amino acid sequence consisting of SEQ ID NO: 46.

In an embodiment, the anti-CLL-1 antibody or fragment thereof may comprise a heavy chain comprising an amino acid sequence consisting of SEQ ID NO: 45; and a light chain comprising an amino acid sequence consisting of SEQ ID NO: 46.

In an embodiment, the anti-CLL-1 antibody or fragment thereof may comprise the sequence of CDRH1, CDRH2 and CDRH3 of the heavy chain variable region and CDRL1, CDRL2 and CDRL3 of the light chain variable region is any one of the followings: (a) CDRH1, CDRH2 and CDRH3 are SEQ ID NOs: 1, 4, and 7, respectively, and CDRL1, CDRL2, and CDRL3 are SEQ ID NOs: 12, 16 and 19, respectively; (b) CDRH1, CDRH2 and CDRH3 are SEQ ID NOs: 2, 5, and 8, respectively, and CDRL1, CDRL2, and CDRL3 are SEQ ID NOs: 13, 17 and 20, respectively; (c) CDRH1, CDRH2 and CDRH3 are SEQ ID NOs: 3, 6, and 9, respectively, and CDRL1, CDRL2, and CDRL3 are SEQ ID NOs: 14, 18 and 21, respectively; (d) CDRH1, CDRH2 and CDRH3 are SEQ ID NOs: 1, 4, and 10, respectively, and CDRL1, CDRL2, and CDRL3 are SEQ ID NOs: 15, 16 and 22, respectively; and (e) CDRH1, CDRH2 and CDRH3 are SEQ ID NOs: 2, 5, and 11, respectively, and CDRL1, CDRL2, and CDRL3 are SEQ ID NOs: 13, 17 and 20, respectively.

In an embodiment, the anti-CLL-1 antibody or antigen-binding fragment thereof may be a mouse antibody, a chimeric antibody, a humanized antibody, or a fully human antibody.

In an embodiment, the anti-CLL-1 antibody or antigen-binding fragment thereof may be selected from a group consisting of a whole IgG, Fab, Fab′, F(ab′)2, xFab, scFab, dsFv, Fv, scFv, scFv-Fc, scFab-Fc, diabody, minibody, scAb, dAb, half-IgG and combinations thereof.

In an embodiment, the anti-CLL-1 antibody or antigen-binding fragment thereof may be in the form of IgG, preferably IgG1.

In an embodiment, the anti-CLL-1 antibody or antigen-binding fragment thereof may be Fab molecule.

In an embodiment, the anti-CLL-1 antibody or antigen-binding fragment thereof may have one or more of the following characteristics: (a) binds to human CLL-1; (b) binds to cynomolgus monkey CLL-1; (c) binds to CLL-1 on the surface of human peripheral blood mononucleocytes (PBMCs); (d) binds to CLL-1 on the surface of cynomolgus monkey PBMCs; and (e) binds to CLL-1 on the surface of a cancer cell.

In an embodiment, a cytotoxic agent may be conjugated to at least portion of the anti-CLL-1 antibody or antigen-binding fragment.

In an embodiment, the cytotoxic agent may be attached to the anti-CLL-1 antibody or antigen-binding fragment through the linker.

In an embodiment, the linker may be cleavable by a protease.

In an embodiment, the anti-CLL-1 antibody may be for use as a medicament.

In an embodiment, the anti-CLL-1 antibody may be for use in treating or preventing a cancer.

Another aspect of the present disclosure provides an immunoconjugate comprising a formula Ab-(L-D)p, wherein: (a) Ab is the anti-CLL-1 antibody of claim 1; (b) L is a linker; (c) D is a cytotoxic agent; and (d) p ranges from 1 to 8.

Another aspect of the present disclosure provides an isolated nucleic acid encoding the anti-CLL-1 antibody.

Another aspect of the present disclosure provides a vector comprising the isolated nucleic acid.

Another aspect of the present disclosure provides a host cell comprising the vector.

Another aspect of the present disclosure provides a pharmaceutical composition comprising the anti-CLL-1 antibody.

Another aspect of the present disclosure provides a method for treating or preventing a cancer in a patient in need thereof, comprising administering to the patient an effective amount of the anti-CLL-1 antibody.

Another aspect of the present disclosure provides a use of the anti-CLL-1 antibody in the manufacture of medicament for treating or preventing a cancer.

Another aspect of the present disclosure provides a use of the anti-CLL-1 antibody for treating or preventing a cancer.

In an embodiment, the pharmaceutical composition may be for treating or preventing a cancer.

In an embodiment, the cancer may be a solid cancer or a blood cancer.

In an embodiment, the cancer may be selected from the group consisting of leukemia, rectal cancer, endometrial cancer, nephroblastoma, basal cell carcinoma, nasopharyngeal cancer, bone tumor, esophageal cancer, lymphoma, Hodgkin's lymphoma, non-Hodgkin's lymphoma, follicular thyroid cancer, hepatocellular carcinoma, oral cancer, renal cell carcinoma, multiple myeloma, mesothelioma, osteosarcoma, myelodysplastic syndrome, mesenchymal tumor, soft tissue sarcoma, liposarcoma, gastrointestinal stromal tumor, malignant peripheral nerve sheath tumor (MPNST), ewing sarcoma, leiomyosarcoma, mesenchymal chondrosarcoma, lymphosarcoma, fibrosarcoma, rhabdomyosarcoma, teratoma, neuroblastoma, medulloblastoma, glioma, benign skin tumor, Burkitt's lymphoma, mantle cell lymphoma, diffuse large B cell lymphoma (DLBCL), follicular lymphoma, marginal zone lymphoma, neuroectodermal tumor, epithelial tumor, cutaneous T-cell lymphoma (CTCL), peripheral T cell lymphoma (PTCL), pancreas cancer, haematological malignancies, kidney cancer, tumor vasculature, breast cancer, renal cancer, ovarian cancer, epithelial ovarian cancer, gastric cancer, liver cancer, lung cancer, colorectal cancer, pancreatic cancer, skin cancer, bladder cancer, testicular cancer, uterine cancer, prostate cancer, small cell lung cancer (SCLC), non-small cell lung cancer (NSCLC), neuroblastoma, brain cancer, colon cancer, squamous cell carcinoma, melanoma, myeloma, cervical cancer, thyroid cancer, head and neck cancer and adrenal cancer.

In an embodiment, the leukemia may be selected from the group consisting of acute lymphoblastic leukemia (ALL), chronic lymphocytic leukemia (CLL), hairy-cell leukemia, myelodysplastic syndrome (MDS), chronic myelogenous leukemia (CML) and acute myeloid leukemia (AML), preferably AML.

In an embodiment, the cancer may be a cancer expressing CLL-1.

### Advantageous Effects

According to the anti-CLL-1 antibody of one aspect, it can bind to CLL-1 with high binding affinity, and can cause activation of T cells, thus it can be used effectively for preventing or treating the cancer expressing CLL-1.

### DESCRIPTION OF DRAWINGS

FIG. 1 is a graph showing ligand binding activity of three chimeric antibodies according to an embodiment using huCLL1-His as a ligand.

FIG. 2 is a graph showing ligand binding activity of three chimeric antibodies according to an embodiment using hFc-huCLL1 as a ligand.

FIG. 3 is a graph showing ligand binding activity of three chimeric antibodies according to an embodiment using 50 ng/well of hFc-Cynomolgus CLL1 as a ligand.

FIG. 4 is a graph showing ligand binding activity of three chimeric antibodies according to an embodiment using 100 ng/well of hFc-Cynomolgus CLL1 as a ligand.

FIG. 5 is a graph showing ligand binding activity of chimeric antibody and humanized antibodies hu16C6 according to an embodiment.

FIG. 6 is a graph showing ligand binding activity of chimeric antibody and humanized antibody hu33C2 according to an embodiment.

FIG. 7 is a graph showing ligand binding activity of various humanized antibodies hu33C2 according to an embodiment.

FIG. 8 is a graph showing cell binding activity of the chimeric antibodies according to an embodiment in CLL1 negative cells and various CLL1-expressing cancer cells.

FIG. 9 is a graph showing cell binding activity of the chimeric antibodies according to an embodiment in HL60.

FIG. 10 is a graph showing cell binding activity of the chimeric antibodies according to an embodiment in U937.

FIG. 11 is a graph showing cell binding activity of the chimeric antibodies according to an embodiment in HEK293E overexpressing cynomolgus CLL-1.

FIG. 12 is a graph showing cell binding activity of the chimeric antibody and various humanized antibodies hu33C2 according to an embodiment.

FIG. 13 is a graph showing cell binding activity of the chimeric antibody and various humanized antibodies hu16C6 according to an embodiment.

FIG. 14 is a graph showing ADCC of ch84A2, hu16C6 and hu33C2 according to an embodiment.

FIG. 15 is a graph showing cell proliferation inhibition activity of the ADCs according to an embodiment in E0L-1.

FIG. 16 is a graph showing cell proliferation inhibition activity of the ADCs according to an embodiment in THP-1.

FIG. 17 is a graph showing cell binding activity of the bispecific antibodies according to an embodiment in CLL1 expressing cancer cells.

FIG. 18 is a graph showing cell binding activity of the bispecific antibodies according to an embodiment.

FIG. 19 is a graph showing T cell activation of the bispecific antibodies according to an embodiment.

FIG. 20 is a graph showing T cell activation and cell lysis activity of the bispecific antibodies according to an embodiment in HL-60.

FIG. 21 is a graph showing T cell activation and cell lysis activity of the bispecific antibodies according to an embodiment in U937.

FIG. 22 is a graph showing antigen-dependent cell lysis activity of the bispecific antibodies according to an embodiment.

FIG. 23 is a graph showing in vivo efficacy of the bipecific antibodies according to an embodiment in U937 xenograft model.

FIG. 24 is images showing BLI (Bioluminescence index) of the administration of the bispecific antibodies according to an embodiment in an HL60-Lu orthotopic AML model.

FIG. 25 is a graph showing results of a quantitative analysis of BLI of the administration of the bispecific antibodies according to an embodiment in HL60-Lu orthotopic AML model (Statistical analysis: Two-way ANOVA (Bonferroni's multiple comparisons test), *p<0.05, **p<0.01, ***p<0.001.).

FIG. 26 is images showing BLI (Bioluminescence index) of the administration of the bispecific antibodies according to an embodiment in an HL60-Lu orthotopic AML model.

FIG. 27 is a graph showing a quantitative analysis of BLI of an administration of the bispecific antibodies according to an embodiment in an HL60-Lu orthotopic AML model (***=P<0.005).

FIG. 28 is graphs showing results of measuring tumor cells in the bone marrow by FACS after the administration of the bispecific antibodies according to an embodiment.

FIG. 29 is images showing results of measuring tumor cells in the bone marrow by IHC staining after the administration of the bispecific antibodies according to an embodiment.

FIG. 30 is graphs showing the activity of the cell lysis of the bispecific antibodies according to an embodiment in AML blasts.

FIG. 31 is graphs showing the activity of the T cell activation of the bispecific antibodies according to an embodiment in AML blasts.

### MODE FOR INVENTION

### Definitions

Unless defined otherwise, technical and scientific terms used herein have the same meaning as generally used in the art to which this invention belongs. For purposes of interpreting this specification, the following definitions will apply and whenever appropriate, terms used in the singular will also include the plural and vice versa.

The term “antibody” herein is used in the broadest sense and encompasses various antibody structures, including but not limited to monoclonal antibodies, polyclonal antibodies, monospecific and multispecific antibodies (e.g., bispecific antibodies), and antibody fragments so long as they exhibit the desired antigen-binding activity.

The term “monoclonal antibody” as used herein refers to an antibody obtained from a population of substantially homogeneous antibodies, i.e., the individual antibodies comprising the population are identical and/or bind the same epitope, except for possible variant antibodies, e.g. containing naturally occurring mutations or arising during production of a monoclonal antibody preparation, such variants generally being present in minor amounts. In contrast to polyclonal antibody preparations, which typically include different antibodies directed against different determinants (epitopes), each monoclonal antibody of a monoclonal antibody preparation is directed against a single determinant on an antigen.

The term “monospecific” antibody as used herein denotes an antibody that has one or more binding sites each of which bind to the same epitope of the same antigen. The term “bispecific” means that the antibody is able to specifically bind to at least two distinct antigenic determinants, for example two binding sites each formed by a pair of an antibody heavy chain variable domain (VH) and an antibody light chain variable domain (VL) binding to different antigens or to different epitopes on the same antigen. Such a bispecific antibody is an 1+1 format. Other bispecific antibody formats are 2+1 or 1+2 formats (comprising two binding sites for a first antigen or epitope and one binding site for a second antigen or epitope) or 2+2 formats (comprising two binding sites for a first antigen or epitope and two binding sites for a second antigen or epitope). Typically, a bispecific antibody comprises two antigen binding sites, each of which is specific for a different antigenic determinant.

The term “valent” as used within the current application denotes the presence of a specified number of binding domains in an antibody or antibody fragment. As such, the terms “monovalent”, “bivalent”, “tetravalent”, and “hexavalent” denote the presence of one binding domain, two binding domains, four binding domains, and six binding domains, respectively, in an antibody.

The bispecific antibodies according to the invention are at least “bivalent” and may be “trivalent” or “multivalent” (e.g. “tetravalent” or “hexavalent”).

In a particular aspect, the antibodies of the present invention have two or more binding sites and are bispecific. That is, the antibodies may be bispecific even in cases where there are more than two binding sites (i.e. that the antibody is trivalent or multivalent).

The terms “full length antibody”, “intact antibody”, and “whole antibody” are used herein interchangeably to refer to an antibody having a structure substantially similar to a native antibody structure. “Native antibodies” refer to naturally occurring immunoglobulin molecules with varying structures. For example, native IgG-class antibodies are heterotetrameric glycoproteins of about 150,000 daltons, composed of two light chains and two heavy chains that are disulfide-bonded. From N- to C-terminus, each heavy chain has a variable region (VH), also called a variable heavy domain or a heavy chain variable domain, followed by three constant domains (CH1, CH2, and CH3), also called a heavy chain constant region. Similarly, from N- to C-terminus, each light chain has a variable region (VL), also called a variable light domain or a light chain variable domain, followed by a light chain constant domain (CL), also called a light chain constant region. The heavy chain of an antibody may be assigned to one of five types, called α (IgA), δ (IgD), ε (IgE), γ (IgG), or μ (IgM), some of which may be further divided into subtypes, e.g. γ1 (IgG1), γ2 (IgG2), γ3 (IgG3), γ4 (IgG4), α1 (IgA1) and α2 (IgA2). The light chain of an antibody may be assigned to one of two types, called kappa (κ) and lambda (λ), based on the amino acid sequence of its constant domain.

As indicated above, the variable region allows the antibody to selectively recognize and specifically bind epitopes on antigens. That is, the VL domain and VH domain, or subset of the complementarity determining regions (CDRs), of an antibody combine to form the variable region that defines a three dimensional antigen-binding site. This quaternary antibody structure forms the antigen-binding site present at the end of each arm of the Y. More specifically, the antigen-binding site is defined by three CDRs on each of the VH and VL chains (i.e. CDR-H1, CDR-H2, CDR-H3, CDR-L1, CDR-L2 and CDR-L3). In some instances, e.g., certain immunoglobulin molecules derived from camelid species or engineered based on camelid immunoglobulins, a complete immunoglobulin molecule may consist of heavy chains only, with no light chains. See, e.g., Hamers-Casterman et al., Nature 363: 446-448 (1993).

The terms “CDR-H” “HCDR” and “CDRH” are used herein interchangeably to refer to a VH chain of the CDR (e.g., CDR-H1, HCDR1 and CDRH1 are refer to a VH1 of the CDR). The terms “CDR-L” “LCDR” and “CDRL” are used herein interchangeably to refer to a VL chain of the CDR (e.g., CDR-L1, LCDR1 and CDRL1 are a refer to a VL1 of the CDR).

In naturally occurring antibodies, the six “complementarity determining regions” or “CDRs” present in each antigen-binding domain are short, non-contiguous sequences of amino acids that are specifically positioned to form the antigen-binding domain as the antibody assumes its three dimensional configuration in an aqueous environment. The remainder of the amino acids in the antigen-binding domains, referred to as “framework” regions, show less inter-molecular variability. The framework regions largely adopt a f-sheet conformation and the CDRs form loops which connect, and in some cases form part of, the f-sheet structure. Thus, framework regions act to form a scaffold that provides for positioning the CDRs in correct orientation by inter-chain, non-covalent interactions. The antigen-binding domain formed by the positioned CDRs defines a surface complementary to the epitope on the immunoreactive antigen. This complementary surface promotes the non-covalent binding of the antibody to its cognate epitope. The amino acids comprising the CDRs and the framework regions, respectively, can be readily identified for any given heavy or light chain variable region by one of ordinary skill in the art, since they have been precisely defined (see www.bioinf.org.uk: Dr. Andrew C.R. Martin's Group; “Sequences of Proteins of Immunological Interest,” Kabat, E., et al., U.S. Department of Health and Human Services, (1983); and Chothia and Lesk, J. Mol. Biol., 196: 901-917 (1987)).

In the case where there are two or more definitions of a term which is used and/or accepted within the art, the definition of the term as used herein is intended to include all such meanings unless explicitly stated to the contrary. A specific example is the use of the term “complementarity determining region” (“CDR”) to describe the non-contiguous antigen combining sites found within the variable region of both heavy and light chain polypeptides. This particular region has been described by Kabat et al., U.S. Dept. of Health and Human Services, “Sequences of Proteins of Immunological Interest” (1983) and by Chothia et al., J. MoI. Biol. 196: 901-917 (1987), which are incorporated herein by reference in their entireties. The CDR definitions according to Kabat and Chothia include overlapping or subsets of amino acid residues when compared against each other. Nevertheless, application of either definition to refer to a CDR of an antibody or variants thereof is intended to be within the scope of the term as defined and used herein. The appropriate amino acid residues which encompass the CDRs as defined by each of the above cited references are set forth in the table below(Table 1) as a comparison. The exact residue numbers which encompass a particular CDR will vary depending on the sequence and size of the CDR. Those skilled in the art can routinely determine which residues comprise a particular CDR given the variable region amino acid sequence of the antibody.

TABLE 1

| | Kabat | Chothia |
|---|---|---|
| CDR-H1 | 31-35 | 26-32 |
| CDR-H2 | 50-65 | 52-58 |
| CDR-H3 | 95-102 | 95-102 |
| CDR-L1 | 24-34 | 26-32 |
| CDR-L2 | 50-56 | 50-52 |
| CDR-L3 | 89-97 | 91-96 |

Kabat et al. also defined a numbering system for variable domain sequences that is applicable to any antibody. One of ordinary skill in the art can unambiguously assign this system of “Kabat numbering” to any variable domain sequence, without reliance on any experimental data beyond the sequence itself. As used herein, “Kabat numbering” refers to the numbering system set forth by Kabat et al., U.S. Dept. of Health and Human Services, “Sequence of Proteins of Immunological Interest” (1983).

Antibodies disclosed herein may be from any animal origin including birds and mammals. Preferably, the antibodies are human, murine, donkey, rabbit, goat, guinea pig, camel, llama, horse, or chicken antibodies.

As used herein, the term “heavy chain constant region” includes amino acid sequences derived from an immunoglobulin heavy chain. As set forth above, it will be understood by one of ordinary skill in the art that the heavy chain constant region may be modified such that they vary in amino acid sequence from the naturally occurring immunoglobulin molecule.

The heavy chain constant region of an antibody disclosed herein may be derived from different immunoglobulin molecules. For example, a heavy chain constant region of a polypeptide may comprise a CH1 domain derived from an IgG1 molecule and a hinge region derived from an IgG3 molecule. In another example, a heavy chain constant region can comprise a hinge region derived, in part, from an IgG1 molecule and, in part, from an IgG3 molecule. In another example, a heavy chain portion can comprise a chimeric hinge derived, in part, from an IgG1 molecule and, in part, from an IgG4 molecule.

As used herein, the term “light chain constant region” includes amino acid sequences derived from antibody light chain. Preferably, the light chain constant region comprises at least one of a constant kappa domain or constant lambda domain.

A “light chain-heavy chain pair” refers to the collection of a light chain and heavy chain that can form a dimer through a disulfide bond between the CL domain of the light chain and the CH1 domain of the heavy chain.

An “antibody fragment” or “antigen-binding fragment” refers to a molecule other than an intact antibody that comprises a portion of an intact antibody that binds the antigen to which the intact antibody binds. An immunologically functional immunoglobulin fragment includes Fab, Fab′, F(ab′)₂, xFab, scFab, dsFv, Fv, scFv, scFv-Fc, scFab-Fc, diabody, minibody, scAb, dAb, half-IgG or combinations thereof, but not limited thereto. The term “Fab” used in Fab, Fab′, F(ab′)₂, xFab and scFab may include a traditional Fab fragment and the chimeric Fab-like domain described in PCT/CN2018/106766 (Wuxibody). In addition, it may be derived from any mammal including human, mouse, rat, camelid or rabbit, but not limited thereto. The functional part of the antibody such as one or more CDRs described herein may be linked with a secondary protein or small molecular compound by a covalent bond, thereby being used as a target therapeutic agent to a specific target. The term “antibody fragment” includes aptamers, spiegelmers, and diabodies. The term “antibody fragment” also includes any synthetic or genetically engineered protein that acts like an antibody by binding to a specific antigen to form a complex.

Antibody fragments can be made by various techniques, including but not limited to proteolytic digestion of an intact antibody as well as production by recombinant host cells (e.g. E. coli or phage), as described herein.

Papain digestion of intact antibodies produces two identical antigen-binding fragments, called “Fab” fragments containing each the heavy- and light-chain variable domains and also the constant domain of the light chain and the first constant domain (CH1) of the heavy chain. As used herein, Thus, the term “Fab fragment” refers to an antibody fragment comprising a light chain fragment comprising a VL domain and a constant domain of a light chain (CL), and a VH domain and a first constant domain (CH1) of a heavy chain. Fab′ fragments differ from Fab fragments by the addition of a few residues at the carboxy terminus of the heavy chain CH1 domain including one or more cysteins from the antibody hinge region. Fab′-SH are Fab′ fragments wherein the cysteine residue(s) of the constant domains bear a free thiol group. Pepsin treatment yields an F(ab′)₂ fragment that has two antigen-combining sites (two Fab fragments) and a part of the Fc region. Herein, “F(ab′)₂ fragment” comprises two light chains, and two heavy chains comprising a variable region, CH1 and a part of a constant region between CH1 and CH2 domains, as aforementioned, and thereby an intrachain disulfide bond between 2 heavy chains is formed. Thus, the F(ab′)₂ fragment consists of two Fab′ fragments, and the two Fab′ fragments are meeting each other by the disulfide bond between them.

The term “cross-Fab fragment” or “xFab fragment” or “crossover Fab fragment” refers to a Fab fragment, wherein either the variable regions or the constant regions of the heavy and light chain are exchanged. Two different chain compositions of a crossover Fab molecule are possible and comprised in the bispecific antibodies of the invention: On the one hand, the variable regions of the Fab heavy and light chain are exchanged, i.e. the crossover Fab molecule comprises a peptide chain composed of the light chain variable region (VL) and the heavy chain constant region (CH1), and a peptide chain composed of the heavy chain variable region (VH) and the light chain constant region (CL). This crossover Fab molecule is also referred to as CrossFab_{(VLVH)}. On the other hand, when the constant regions of the Fab heavy and light chain are exchanged, the crossover Fab molecule comprises a peptide chain composed of the heavy chain variable region (VH) and the light chain constant region (CL), and a peptide chain composed of the light chain variable region (VL) and the heavy chain constant region (CH1). This crossover Fab molecule is also referred to as CrossFab_{(CLCH1)}.

A “single chain Fab fragment” or “scFab” is a polypeptide consisting of an antibody heavy chain variable domain (VH), an antibody constant domain 1 (CH1), an antibody light chain variable domain (VL), an antibody light chain constant domain (CL) and a linker, wherein said antibody domains and said linker have one of the following orders in N-terminal to C-terminal direction: a) VH-CH1-linker-VL-CL, b) VL-CL-linker-VH-CH1, c) VH-CL-linker-VL-CH1 or d) VL-CH1-linker-VH-CL; and wherein said linker is a polypeptide of at least 30 amino acids, preferably between 32 and 50 amino acids. Said single chain Fab fragments are stabilized via the natural disulfide bond between the CL domain and the CH1 domain. In addition, these single chain Fab molecules might be further stabilized by generation of interchain disulfide bonds via insertion of cysteine residues (e.g. position 44 in the variable heavy chain and position 100 in the variable light chain according to Kabat numbering).

A “crossover single chain Fab fragment” or “x-scFab” is a is a polypeptide consisting of an antibody heavy chain variable domain (VH), an antibody constant domain 1 (CH1), an antibody light chain variable domain (VL), an antibody light chain constant domain (CL) and a linker, wherein said antibody domains and said linker have one of the following orders in N-terminal to C-terminal direction: a) VH-CL-linker-VL-CH1 and b) VL-CH1-linker-VH-CL; wherein VH and VL form together an antigen binding domain which binds specifically to an antigen and wherein said linker is a polypeptide of at least 30 amino acids. In addition, these x-scFab molecules might be further stabilized by generation of interchain disulfide bonds via insertion of cysteine residues (e.g. position 44 in the variable heavy chain and position 100 in the variable light chain according to Kabat numbering).

A “Fv region” is an antibody which comprises each variable region of a heavy chain and a light chain, but does not comprise a constant region. scFv is one that Fv is linked by a flexible linker. scFv-Fc is one that Fc is linked to scFv. The minibody is one that CH3 is linked to scFv. The diabody comprises two molecules of scFv. A “single-chain variable fragment” or “scFv” refers to a fusion protein of the variable regions of the heavy (VH) and light chains (VL) of immunoglobulins. In some aspects, the regions are connected with a short linker peptide of ten to about 25 amino acids. The linker can be rich in glycine for flexibility, as well as serine or threonine for solubility, and can either connect the N-terminus of the VH with the C-terminus of the VL, or vice versa. This protein retains the specificity of the original immunoglobulin, despite removal of the constant regions and the introduction of the linker. ScFv molecules are known in the art and are described, e.g., in U.S. Pat. No. 5,892,019.

A “short-chain antibody (scAb)” is a single polypeptide chain comprising one variable region of a heavy chain or a light chain constant region in which a heavy chain and light chain variable region is linked by a flexible linker. The short-chain antibody may refer to for example, U.S. Pat. No. 5,260,203, and this is disclosed herein by reference.

A “domain antibody (dAb)” is an immunologically functional immunoglobulin fragment comprising a variable region of heavy chain or a variable region of light chain only. In one embodiment, two or more of VH regions are linked by a covalent bond by a peptide linker, to form a bivalent domain antibody. Two VH regions of this bivalent domain antibody may target the same or different antigen.

The term “full length IgG” according to the invention is defined as comprising an essentially complete IgG, which however does not necessarily have all functions of an intact IgG. For the avoidance of doubt, a full length IgG contains two heavy and two light chains. Each chain contains constant (C) and variable (V) regions, which can be broken down into domains designated CH1, CH2, CH3, VH, and CL, VL. An IgG antibody binds to antigen via the variable region domains contained in the Fab portion, and after binding can interact with molecules and cells of the immune system through the constant domains, mostly through the Fc portion. The terms ‘variable region domain’, ‘variable region’, ‘variable domain’, ‘VH/VL pair’, ‘VH/VL’, ‘Fab portion’, ‘Fab arm’, ‘Fab′ or ‘arm’ are used herein interchangeably. Full length antibodies according to the invention encompass IgG molecules wherein mutations may be present that provide desired characteristics. Such mutations should not be deletions of substantial portions of any of the regions. However, IgG molecules wherein one or several amino acid residues are deleted, without essentially altering the binding characteristics of the resulting IgG molecule, are embraced within the term “full length IgG”. For instance, such IgG molecules can have one or more deletions of between 1 and 10 amino acid residues, preferably in non-CDR regions, wherein the deleted amino acids are not essential for the binding specificity of the IgG.

Full length IgG antibodies are preferred because of their favourable half life and the need to stay as close to fully autologous (human) molecules for reasons of immunogenicity. According to the invention, bispecific IgG antibodies are used. In a preferred embodiment, bispecific full length IgG1 antibodies are used. IgG1 is favoured based on its long circulatory half life in man. In order to prevent any immunogenicity in humans it is preferred that the bispecific IgG antibody according to the invention is a human IgG1. The term ‘bispecific’ (bs) means that one arm of the antibody binds to a first antigen whereas the second arm binds to a second antigen, wherein said first and second antigens are not identical. According to the present invention, said first and second antigens are in fact two different molecules that are located on two different cell types. The term ‘one arm [of the antibody]’ preferably means one Fab portion of the full length IgG antibody. Bispecific antibodies that mediate cytotoxicity by recruiting and activating endogenous immune cells are an emerging class of next-generation antibody therapeutics. This can be achieved by combining antigen binding specificities for target cells (i.e., tumor cells) and effector cells (i.e., T cells, NK cells, and macrophages) in one molecule (Cui et al. JBC 2012 (287) 28206-28214; Kontermann, MABS 2012 (4) 182-197; Chames and Baty, MABS 2009 (1) 539-547; Moore et al. Blood 2011 (117) 4542-4551; Loffler et al. 2000 Blood 95:2098; Zeidler et al. 1999 J. Immunol. 163:1246). According to the invention, bispecific antibodies are provided wherein one arm binds the CLL-1 antigen on aberrant (tumor) cells whereas the second arm binds an antigen on immune effector cells.

As used herein, the term “antigen binding domain” or “antigen-binding site” refers to the part of the antibody or antibody fragment that specifically binds to an antigenic determinant. More particularly, the term “antigen-binding domain” refers the part of an antibody that comprises the area which specifically binds to and is complementary to part or all of an antigen. Where an antigen is large, an antibody or antibody fragment may only bind to a particular part of the antigen, which part is termed an epitope. An antigen binding domain may be provided by, for example, one or more variable domains (also called variable regions). Preferably, an antigen binding domain comprises an antibody light chain variable region (VL) and an antibody heavy chain variable region (VH). In one aspect, the antigen binding domain is able to bind to its antigen and block or partly block its function. Antigen binding domains that specifically bind to CCL-1 or to CD3 include antibodies and fragments thereof as further defined herein. In addition, antigen binding domains may include scaffold antigen binding proteins, e.g. binding domains which are based on designed repeat proteins or designed repeat domains (see e.g. WO 2002/020565).

As used herein, the term “antigenic determinant” is synonymous with “antigen” and “epitope” and refers to a site (e.g. a contiguous stretch of amino acids or a conformational configuration made up of different regions of non-contiguous amino acids) on a polypeptide macromolecule to which an antigen binding moiety binds, forming an antigen binding moiety-antigen complex. Useful antigenic determinants can be found, for example, on the surfaces of tumor cells, on the surfaces of virus-infected cells, on the surfaces of other diseased cells, on the surface of immune cells, free in blood serum, and/or in the extracellular matrix (ECM). The proteins useful as antigens herein can be any native form the proteins from any vertebrate source, including mammals such as primates (e.g. humans) and rodents (e.g. mice and rats), unless otherwise indicated. In a particular embodiment the antigen is a human protein. Where reference is made to a specific protein herein, the term encompasses the “full-length”, unprocessed protein as well as any form of the protein that results from processing in the cell. The term also encompasses naturally occurring variants of the protein, e.g. splice variants or allelic variants.

By “specific binding” is meant that the binding is selective for the antigen and can be discriminated from unwanted or non-specific interactions. The ability of an antibody or antibody fragment to bind to a specific antigen can be measured either through an enzyme-linked immunosorbent assay (ELISA) or other techniques familiar to one of skill in the art, e.g. Surface Plasmon Resonance (SPR) technique (analyzed on a BIAcore instrument) (Liljeblad et al., Glyco J 17, 323-329 (2000)), and traditional binding assays (Heeley, Endocr Res 28, 217-229 (2002)).

“Affinity” or “binding affinity” refers to the strength of the sum total of non-covalent interactions between a single binding site of a molecule (e.g. an antibody) and its binding partner (e.g. an antigen). Unless indicated otherwise, as used herein, “binding affinity” refers to intrinsic binding affinity which reflects a 1:1 interaction between members of a binding pair (e.g. antibody and antigen). The affinity of a molecule X for its partner Y can generally be represented by the dissociation constant (Kd), which is the ratio of dissociation and association rate constants (koff and kon, respectively). Thus, equivalent affinities may comprise different rate constants, as long as the ratio of the rate constants remains the same. Affinity can be measured by common methods known in the art, including those described herein. A particular method for measuring affinity is Surface Plasmon Resonance (SPR).

As used herein, the term “high affinity” of an antibody refers to an antibody having a Kd of 10-9 M or less and even more particularly 10-1° M or less for a target antigen. The term “low affinity” of an antibody refers to an antibody having a Kd of 10-8 or higher.

An “affinity matured” antibody refers to an antibody with one or more alterations in one or more hypervariable regions (HVRs), compared to a parent antibody which does not possess such alterations, such alterations resulting in an improvement in the affinity of the antibody for antigen.

The term “a bispecific antibody that specifically binds CLL-1 and CD3” “bispecific antibody specific for CLL-1 and CD3” or an “anti-CLL-1/anti-CD3 antibody” are used interchangeably herein and refer to a bispecific antibody that is capable of binding CLL-1 and CD3 with sufficient affinity such that the antibody is useful as a diagnostic and/or therapeutic agent in targeting CLL-1 and CD3.

The term “C-type lectin-like molecule-1 (CLL-1)”, also known as MICL or CLEC12A, is a type II transmembrane glycoprotein and member of the large family of C-type lectin-like receptors involved in immune regulation. CLL-1 has previously been identified from myeloid-derived cells. The intracellular domain of CLL-1 contains an immunotyrosine-based inhibition motif (ITIM) and a YXXM motif. Phosphorylation of ITIM-containing receptors on a variety of cells results in inhibition of activation pathways through recruitment of protein tyrosine phosphatases SHP-1, SHP-2 and SHIP. The YXXM motif has a potential SH2 domain-binding site for the p85 sub-unit of PI-3 kinase, 13 which has been implicated in cellular activation pathways, revealing a potential dual role of CLL-1 as an inhibitory and activating molecule on myeloid cells. Indeed, association of CLL-1 with SHP-1 and SHP-2 has been demonstrated experimentally in transfected and myeloid-derived cell lines.

The pattern of expression of CLL-1 in hematopoietic cells is restricted. It is found in particular in myeloid cells derived from peripheral blood and bone marrow, as well as in the majority of AML blasts. A recent study indicated that CLL-1 is also present on the majority of leukemic stem cells in the CD34+/CD38− compartment in AML but absent from CD34+/CD38- cells in normal and in regenerating bone marrow controls, which aids the discrimination between normal and leukemic stem cells. (See, e.g., Zhao et al., Haematologica 95:71-78 (2010); Bakker et al., Cancer Res. 64:8443-8450 (2004)). The nucleotide and protein sequences of CLL-1 are known for many species. For example, the human sequences can be found at Genbank accession number AF247788.1 and Uniprot accession number Q5QGZ9.

The terms “anti-CCL-1 antibody”, “an antibody that binds to CCL-1” and “an antibody comprising an antigen binding domain that binds to CCL-1” refer to an antibody that is capable of binding CCL-1, especially a CCL-1 polypeptide expressed on a cell surface, with sufficient affinity such that the antibody is useful as a diagnostic and/or therapeutic agent in targeting CCL-1. In one aspect, the extent of binding of an anti-CCL-1 antibody to an unrelated, non-CCL-1 protein is less than about 10% of the binding of the antibody to CCL-1 as measured, e.g., by radioimmunoassay (RIA) or flow cytometry (FACS) or by a Surface Plasmon Resonance assay using a biosensor system such as a Biacore) system. In certain aspects, an antigen binding protein that binds to human CCL-1 has a K_{D} value of the binding affinity for binding to human PD1 of ≤1 μM, ≤100 nM, ≤10 nM, ≤1 nM, ≤0.1 nM, ≤0.01 nM, or ≤0.001 nM (e.g. 10⁻⁸ M or less, e.g. from 10⁻⁸ M to 10⁻¹³ M, e.g., from 10⁻⁹M to 10-¹³M). The term “anti-CCL-1 antibody” also encompasses bispecific antibodies that are capable of binding CCL-1 and a different antigen.

The terms “CLL-1” and “CLL1” are used herein interchangeably.

The term “immune effector cell” or “effector cell” as used herein refers to a cell within the natural repertoire of cells in the mammalian immune system which can be activated to affect the viability of a target cell. Immune effector cells include cells of the lymphoid lineage such as natural killer (NK) cells, T cells including cytotoxic T cells, or B cells, but also cells of the myeloid lineage can be regarded as immune effector cells, such as monocytes or macrophages, dendritic cells and neutrophilic granulocytes. Hence, said effector cell is preferably an NK cell, a T cell, a B cell, a monocyte, a macrophage, a dendritic cell or a neutrophilic granulocyte. According to the invention, recruitment of effector cells to aberrant cells means that immune effector cells are brought in close vicinity to the aberrant target cells cells such that the effector cells can directly kill, or indirectly initiate the killing of the aberrant cells that they are recruited to. In order to avoid non specific interactions it is preferred that the bispecific antibodies of the invention specifically recognize antigens on immune effector cells that are at least over-expressed by these immune effector cells compared to other cells in the body. Target antigens present on immune effector cells may include CD3, CD16, CD25, CD28, CD64, CD89, NKG2D and NKp46, Preferably, the antigen on immune effector cells is CD3 expressed on T cells, or a functional equivalent thereof (a functional equivalent would be a CD3-like molecule with a similar distribution on T-cells and a similar function (in kind, not necessarily in amount)). As used herein, the term “CD3” also encompasses functional equivalents of CD3. The most preferred antigen on an immune effector cell is the CD3 C chain. This antigen has been shown to be very effective in recruiting T cells to aberrant cells. Hence, a bispecific IgG antibody according to the present invention preferably contains one arm that specifically recognizes CD3ε.

The term “CD3” refers to any native CD3 from any vertebrate source, including mammals such as primates (e.g. humans), non-human primates (e.g. cynomolgus monkeys) and rodents (e.g. mice and rats), unless otherwise indicated.

The term encompasses “full-length,” unprocessed CD3 as well as any form of CD3 that results from processing in the cell. The term also encompasses naturally occurring variants of CD3, e.g., splice variants or allelic variants. In one embodiment, CD3 is human CD3, particularly the epsilon sub-unit of human CD3 (CD3c). The amino acid sequence of human CD3c is shown in UniProt (www.uniprot.org) accession no. P07766 (version 189), or NCBI (www.ncbi.nlm.nih.gov/) RefSeq NP_000724.1. The amino acid sequence of cynomolgus [Macaca fascicularis]CD3ε is shown in NCBI GenBank no. BAB71849.1.

The terms “anti-CD3 antibody”, “an antibody that binds to CD3” and “an antibody comprising an antigen binding domain that binds to CD3” refer to an antibody that is capable of binding CD3 with sufficient affinity such that the antibody is useful as a diagnostic and/or therapeutic agent in targeting CD3. In one aspect, the extent of binding of an anti-CD3 antibody to an unrelated, non-CD3 protein is less than about 10% of the binding of the antibody to CD3 as measured, e.g., by a radioimmunoassay (RIA). In certain embodiments, an antibody that binds to CD3 has a dissociation constant (Kd) of ≤1 μM, ≤100 nM, ≤10 nM, ≤1 nM, ≤0.1 nM, ≤0.01 nM, or ≤0.001 nM (e.g. 10⁻⁸ M or less, e.g. from 10⁻⁸ M to 10⁻¹³ M, e.g., from 10⁻⁹ M to 10⁻¹³ M). In certain aspects, an anti-CD3 antibody binds to an epitope of CD3 that is conserved among CD3 from different species. The term “anti-CD3 antibody” also encompasses bispecific antibodies that are capable of binding CD3 and a different antigen.

The term “mouse” antibody is intended to include antibodies having variable regions in which both the framework and CDR regions are derived from mouse germline immunoglobulin sequences. Furthermore, if the antibody contains a constant region, the constant region also is derived from mouse germline immunoglobulin sequences. The mouse antibodies of the disclosure can include amino acid residues not encoded by mouse germline immunoglobulin sequences (e.g., mutations introduced by random or site-specific mutagenesis in vitro or by somatic mutation in vivo).

The term “chimeric” antibody refers to an antibody in which a portion of the heavy and/or light chain is derived from a particular source or species, while the remainder of the heavy and/or light chain is derived from a different source or species.

The “class” of an antibody refers to the type of constant domain or constant region possessed by its heavy chain. There are five major classes of antibodies: IgA, IgD, IgE, IgG, and IgM, and several of these may be further divided into subclasses (isotypes), e.g. IgG₁, IgG₂, IgG₃, IgG₄, IgA₁, and IgA₂. The heavy chain constant domains that correspond to the different classes of immunoglobulins are called α, δ, ε, γ, and u respectively.

A “humanized” antibody refers to a chimeric antibody comprising amino acid residues from non-human HVRs and amino acid residues from human FRs. In certain embodiments, a humanized antibody will comprise substantially all of at least one, and typically two, variable domains, in which all or substantially all of the HVRs (e.g., CDRs) correspond to those of a non-human antibody, and all or substantially all of the FRs correspond to those of a human antibody.

A humanized antibody optionally may comprise at least a portion of an antibody constant region derived from a human antibody. A “humanized form” of an antibody, e.g., a non-human antibody, refers to an antibody that has undergone humanization. Other forms of “humanized antibodies” encompassed by the present invention are those in which the constant region has been additionally modified or changed from that of the original antibody to generate the properties according to the invention, especially in regard to C1q binding and/or Fc receptor (FcR) binding.

A “human” antibody is one which possesses an amino acid sequence which corresponds to that of an antibody produced by a human or a human cell or derived from a non-human source that utilizes human antibody repertoires or other human antibody-encoding sequences. This definition of a human antibody specifically excludes a humanized antibody comprising non-human antigen-binding residues.

The term “Fc domain” or “Fc region” herein is used to define a C-terminal region of an immunoglobulin heavy chain that contains at least a portion of the constant region. The term includes native sequence Fc regions and variant Fc regions. Although the boundaries of the Fc region of an IgG heavy chain might vary slightly, the human IgG heavy chain Fc region is usually defined to extend from Cys226, or from Pro230, to the carboxyl-terminus of the heavy chain. However, antibodies produced by host cells may undergo post-translational cleavage of one or more, particularly one or two, amino acids from the C-terminus of the heavy chain. Therefore an antibody produced by a host cell by expression of a specific nucleic acid molecule encoding a full-length heavy chain may include the full-length heavy chain, or it may include a cleaved variant of the full-length heavy chain (also referred to herein as a “cleaved variant heavy chain”). This may be the case where the final two C-terminal amino acids of the heavy chain are glycine (G446) and lysine (K447, numbering according to Kabat EU index). Therefore, the C-terminal lysine (Lys447), or the C-terminal glycine (Gly446) and lysine (K447), of the Fc region may or may not be present. Amino acid sequences of heavy chains including Fc domains (or a sub-unit of an Fc domain as defined herein) are denoted herein without C-terminal glycine-lysine dipeptide if not indicated otherwise. In one embodiment of the invention, a heavy chain including a sub-unit of an Fc domain as specified herein, comprised in an antibody or bispecific antibody according to the invention, comprises an additional C-terminal glycine-lysine dipeptide (G446 and K447, numbering according to EU index of Kabat). In one embodiment of the invention, a heavy chain including a sub-unit of an Fc domain as specified herein, comprised in an antibody or bispecific antibody according to the invention, comprises an additional C-terminal glycine residue (G446, numbering according to EU index of Kabat). Compositions of the invention, such as the pharmaceutical compositions described herein, comprise a population of antibodies or bispecific antibodies of the invention. The population of antibodies or bispecific antibodies may comprise molecules having a full-length heavy chain and molecules having a cleaved variant heavy chain. The population of antibodies or bispecific antibodies may consist of a mixture of molecules having a full-length heavy chain and molecules having a cleaved variant heavy chain, wherein at least 50%, at least 60%, at least 70%, at least 80% or at least 90% of the antibodies or bispecific antibodies have a cleaved variant heavy chain. In one embodiment of the invention a composition comprising a population of antibodies or bispecific antibodies of the invention comprises an antibody or bispecific antibody comprising a heavy chain including a sub-unit of an Fc domain as specified herein with an additional C-terminal glycine-lysine dipeptide (G446 and K447, numbering according to EU index of Kabat). In one embodiment of the invention a composition comprising a population of antibodies or bispecific antibodies of the invention comprises an antibody or bispecific antibody comprising a heavy chain including a sub-unit of an Fc domain as specified herein with an additional C-terminal glycine residue (G446, numbering according to EU index of Kabat). In one embodiment of the invention such a composition comprises a population of antibodies or bispecific antibodies comprised of molecules comprising a heavy chain including a sub-unit of an Fc domain as specified herein; molecules comprising a heavy chain including a sub-unit of a Fc domain as specified herein with an additional C-terminal glycine residue (G446, numbering according to EU index of Kabat); and molecules comprising a heavy chain including a sub-unit of an Fc domain as specified herein with an additional C-terminal glycine-lysine dipeptide (G446 and K447, numbering according to EU index of Kabat). Unless otherwise specified herein, numbering of amino acid residues in the Fc region or constant region is according to the EU numbering system, also called the EU index, as described in Kabat et al., Sequences of Proteins of Immunological Interest, 5th Ed. Public Health Service, National Institutes of Health, Bethesda, Md., 1991 (see also above). A “sub-unit” of an Fc domain as used herein refers to one of the two polypeptides forming the dimeric Fc domain, i.e. a polypeptide comprising C-terminal constant regions of an immunoglobulin heavy chain, capable of stable self-association. For example, a sub-unit of an IgG Fc domain comprises an IgG CH2 and an IgG CH3 constant domain.

A “modification promoting the association of the first and the second sub-unit of the Fc domain” is a manipulation of the peptide backbone or the post-translational modifications of an Fc domain sub-unit that reduces or prevents the association of a polypeptide comprising the Fc domain sub-unit with an identical polypeptide to form a homodimer. A modification promoting association as used herein particularly includes separate modifications made to each of the two Fc domain sub-units desired to associate (i.e. the first and the second sub-unit of the Fc domain), wherein the modifications are complementary to each other so as to promote association of the two Fc domain sub-units. For example, a modification promoting association may alter the structure or charge of one or both of the Fc domain sub-units so as to make their association sterically or electrostatically favorable, respectively. Thus, (hetero)dimerization occurs between a polypeptide comprising the first Fc domain sub-unit and a polypeptide comprising the second Fc domain sub-unit, which might be non-identical in the sense that further components fused to each of the sub-units (e.g. antigen binding moieties) are not the same. In some embodiments the modification promoting association comprises an amino acid mutation in the Fc domain, specifically an amino acid substitution. In a particular embodiment, the modification promoting association comprises a separate amino acid mutation, specifically an amino acid substitution, in each of the two sub-units of the Fc domain.

The term “peptide linker” refers to a peptide comprising one or more amino acids, typically about 2 to 20 amino acids. Peptide linkers are known in the art or are described herein. Suitable, non-immunogenic linker peptides are, for example, (G₄S)ₙ, (SG₄)ₙ or G₄(SG₄)ₙ peptide linkers, wherein “n” is generally a number between 1 and 10, typically between 2 and 4, in particular 2, i.e. the peptides selected from the group consisting of GGGGS (SEQ ID NO: 58) GGGGSGGGGS (SEQ ID NO: 59), SGGGGSGGGG (SEQ ID NO: 60) and GGGGSGGGGSGGGG (SEQ ID NO: 61), but also include the sequences GSPGSSSSGS (SEQ ID NO: 62), GGGGSGGGGSGGGGS (SEQ ID NO: 63), GSGSGSGS (SEQ ID NO: 64), GSGSGNGS (SEQ ID NO: 65), GGSGSGSG (SEQ ID NO: 66), GGSGSG (SEQ ID NO: 67), GGSG (SEQ ID NO: 68), GGSGNGSG (SEQ ID NO: 69), GGNGSGSG (SEQ ID NO: 70) and GGNGSG (SEQ ID NO: 71).

The term “effector functions” refers to those biological activities attributable to the Fc region of an antibody, which vary with the antibody isotype. Examples of antibody effector functions include: C1q binding and complement dependent cytotoxicity (CDC), Fc receptor binding, antibody-dependent cell-mediated cytotoxicity (ADCC), antibody-dependent cellular phagocytosis (ADCP), cytokine secretion, immune complex-mediated antigen uptake by antigen presenting cells, down regulation of cell surface receptors (e.g. B cell receptor), and B cell activation.

As used herein, the terms “engineer, engineered, engineering” are considered to include any manipulation of the peptide backbone or the post-translational modifications of a naturally occurring or recombinant polypeptide or fragment thereof. Engineering includes modifications of the amino acid sequence, of the glycosylation pattern, or of the side chain group of individual amino acids, as well as combinations of these approaches.

The term “amino acid mutation” as used herein is meant to encompass amino acid substitutions, deletions, insertions, and modifications. Any combination of substitution, deletion, insertion, and modification can be made to arrive at the final construct, provided that the final construct possesses the desired characteristics, e.g., reduced binding to an Fc receptor, or increased association with another peptide. Amino acid sequence deletions and insertions include amino- and/or carboxy-terminal deletions and insertions of amino acids. Particular amino acid mutations are amino acid substitutions. For the purpose of altering e.g. the binding characteristics of an Fc region, non-conservative amino acid substitutions, i.e. replacing one amino acid with another amino acid having different structural and/or chemical properties, are particularly preferred. Amino acid substitutions include replacement by non-naturally occurring amino acids or by naturally occurring amino acid derivatives of the twenty standard amino acids (e.g. 4-hydroxyproline, 3-methylhistidine, ornithine, homoserine, 5-hydroxylysine). Amino acid mutations can be generated using genetic or chemical methods well known in the art. Genetic methods may include site-directed mutagenesis, PCR, gene synthesis and the like. It is contemplated that methods of altering the side chain group of an amino acid by methods other than genetic engineering, such as chemical modification, may also be useful. Various designations may be used herein to indicate the same amino acid mutation. For example, a substitution from proline at position 329 of the Fc domain to glycine can be indicated as 329G, G329, G329, P329G, or Pro329Gly.

“Percent (%) amino acid sequence identity” with respect to a reference polypeptide sequence is defined as the percentage of amino acid residues in a candidate sequence that are identical with the amino acid residues in the reference polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, Clustal W, Megalign (DNASTAR) software or the FASTA program package. Those skilled in the art can determine appropriate parameters for aligning sequences, including any algorithms needed to achieve maximal alignment over the full length of the sequences being compared. For purposes herein, however, % amino acid sequence identity values are generated using the ggsearch program of the FASTA package version 36.3.8c or later with a BLOSUM50 comparison matrix. The FASTA program package was authored by W. R. Pearson and D. J. Lipman (1988), “Improved Tools for Biological Sequence Analysis”, PNAS 85:2444-2448; W. R. Pearson (1996) “Effective protein sequence comparison” Meth. Enzymol. 266:227-258; and Pearson et. al. (1997) Genomics 46:24-36, and is publicly available from http://fasta.bioch.virginia.edu/fasta_www2/fasta_down.shtml. Alternatively, a public server accessible at http://fasta.bioch.virginia.edu/fasta_www2/index.cgi can be used to compare the sequences, using the ggsearch (global protein:protein) program and default options (BLOSUM50; open: −10; ext: −2; Ktup=2) to ensure a global, rather than local, alignment is performed. Percent amino acid identity is given in the output alignment header.

An “immunoconjugate” is an antibody conjugated to one or more heterologous molecule(s), including but not limited to a cytotoxic agent.

As used herein, the term “polypeptide” is intended to encompass a singular “polypeptide” as well as plural “polypeptides,” and refers to a molecule composed of monomers (amino acids) linearly linked by amide bonds (also known as peptide bonds). The term “polypeptide” refers to any chain or chains of two or more amino acids, and does not refer to a specific length of the product. Thus, peptides, dipeptides, tripeptides, oligopeptides, “protein,” “amino acid chain,” or any other term used to refer to a chain or chains of two or more amino acids, are included within the definition of “polypeptide,” and the term “polypeptide” may be used instead of, or interchangeably with any of these terms. The term “polypeptide” is also intended to refer to the products of post-expression modifications of the polypeptide, including without limitation glycosylation, acetylation, phosphorylation, amidation, derivatization by known protecting/blocking groups, proteolytic cleavage, or modification by non-naturally occurring amino acids. A polypeptide may be derived from a natural biological source or produced by recombinant technology, but is not necessarily translated from a designated nucleic acid sequence. It may be generated in any manner, including by chemical synthesis. The term “polypeptide” also encompass a variant of a polypeptide and derivative of a polypeptide. Moreover, “polypeptide fragment” means a polypeptide having deletion of an amino acid sequence of an amino terminal, deletion of an amino acid sequence of a carboxyl terminal and/or an internal deletion, compared to a full-length protein. This fragment may also include modified amino acids compared to a full-length protein. In one embodiment, the fragment may be about 5 to 900 amino acids in length, for example, at least 5, 6, 8, 10, 14, 20, 50, 70, 100, 110, 150, 200, 250, 300, 350, 400, 450, 500, 550, 600, 650, 700, 750, 800, 850 or more amino acids in length. Considering the purpose of the present invention, the useful polypeptide fragment includes an immunological functional fragment of an antibody comprising an antigen-binding domain. In case of CLL-1 or CD3 binding antibody, such a useful fragment includes a CDR sequence comprising 1, 2, or 3 of heavy chains or light chains, or all or a portion of antibody chain comprising a variable region or constant region of a heavy chain or light chain, but not limited thereto.

As used herein, “variant” of a polypeptide such as for example, an antigen-binding fragment, a protein or an antibody is a polypeptide in which one or more amino acid residues are inserted, deleted, added and/or substituted, as compared to another polypeptide sequence, and includes a fusion polypeptide. In addition, a protein variant includes one modified by protein enzyme cutting, phosphorylation or other posttranslational modification, but maintaining biological activity of the antibody disclosed herein, for example, specific binding to CLL-1 and biological activity. The variant may be about 99%, 98%, 97%, 96%, 95%, 94%, 93%, 92%, 91%, 90%, 89%, 88%, 87%, 86%, 85%, 84%, 83%, 82%, 81%, or 80% identical to the sequence of the antibody or its antigen-binding fragment disclosed herein.

As used herein, the term “derivative” of the polypeptide means a polypeptide chemically modified through conjugation with other chemical moiety, which is different from an insertion, deletion, addition or substitution variant.

As used herein, the term “recombinant” as it pertains to polypeptides or polynucleotides intends a form of the polypeptide or polynucleotide that does not exist naturally, a non-limiting example of which can be created by combining polynucleotides or polypeptides that would not normally occur together.

“Homology” or “identity” or “similarity” refers to sequence similarity between two peptides or between two nucleic acid molecules. Homology can be determined by comparing a position in each sequence which may be aligned for purposes of comparison. When a position in the compared sequence is occupied by the same base or amino acid, then the molecules are homologous at that position. A degree of homology between sequences is a function of the number of matching or homologous positions shared by the sequences. An “unrelated” or “non-homologous” sequence shares less than 40% identity, though preferably less than 25% identity, with one of the sequences of the present disclosure.

A polynucleotide or polynucleotide region (or a polypeptide or polypeptide region) has a certain percentage (for example, 60%, 65%, 70%, 75%, 80%, 85%, 90%, 95%, 98% or 99%) of “sequence identity” to another sequence means that, when aligned, that percentage of bases (or amino acids) are the same in comparing the two sequences.

The term “polynucleotide” refers to an isolated nucleic acid molecule or construct, e.g. messenger RNA (mRNA), virally-derived RNA, or plasmid DNA (pDNA). A polynucleotide may comprise a conventional phosphodiester bond or a non-conventional bond (e.g. an amide bond, such as found in peptide nucleic acids (PNA). The term “nucleic acid molecule” refers to any one or more nucleic acid segments, e.g. DNA or RNA fragments, present in a polynucleotide.

By “isolated” nucleic acid molecule or polynucleotide is intended a nucleic acid molecule, DNA or RNA, which has been removed from its native environment. For example, a recombinant polynucleotide encoding a polypeptide contained in a vector is considered isolated for the purposes of the present invention. Further examples of an isolated polynucleotide include recombinant polynucleotides maintained in heterologous host cells or purified (partially or substantially) polynucleotides in solution. An isolated polynucleotide includes a polynucleotide molecule contained in cells that ordinarily contain the polynucleotide molecule, but the polynucleotide molecule is present extrachromosomally or at a chromosomal location that is different from its natural chromosomal location. Isolated RNA molecules include in vivo or in vitro RNA transcripts of the present invention, as well as positive and negative strand forms, and double-stranded forms. Isolated polynucleotides or nucleic acids according to the present invention further include such molecules produced synthetically. In addition, a polynucleotide or a nucleic acid may be or may include a regulatory element such as a promoter, ribosome binding site, or a transcription terminator. The term “isolated” as used herein also refers to a nucleic acid or peptide that is substantially free of cellular material, viral material, or culture medium when produced by recombinant DNA techniques, or chemical precursors or other chemicals when chemically synthesized. The term “isolated” is also used herein to refer to cells or polypeptides which are isolated from other cellular proteins or tissues. Isolated polypeptides are meant to encompass both purified and recombinant polypeptides. “Isolated polynucleotide (or nucleic acid) encoding [e.g. an antibody or bispecific antibody of the invention]” refers to one or more polynucleotide molecules encoding antibody heavy and light chains (or fragments thereof), including such polynucleotide molecule(s) in a single vector or separate vectors, and such nucleic acid molecule(s) present at one or more locations in a host cell.

The term “expression cassette” refers to a polynucleotide generated recombinantly or synthetically, with a series of specified nucleic acid elements that permit transcription of a particular nucleic acid in a target cell. The recombinant expression cassette can be incorporated into a plasmid, chromosome, mitochondrial DNA, plastid DNA, virus, or nucleic acid fragment. Typically, the recombinant expression cassette portion of an expression vector includes, among other sequences, a nucleic acid sequence to be transcribed and a promoter. In certain embodiments, the expression cassette comprises polynucleotide sequences that encode antibodies or bispecific antibodies of the invention or fragments thereof.

The term “vector” or “expression vector” refers to a DNA molecule that is used to introduce and direct the expression of a specific gene to which it is operably associated in a cell. The term includes the vector as a self-replicating nucleic acid structure as well as the vector incorporated into the genome of a host cell into which it has been introduced. The expression vector of the present invention comprises an expression cassette. Expression vectors allow transcription of large amounts of stable mRNA. Once the expression vector is inside the cell, the ribonucleic acid molecule or protein that is encoded by the gene is produced by the cellular transcription and/or translation machinery. In one embodiment, the expression vector of the invention comprises an expression cassette that comprises polynucleotide sequences that encode antibodies or bispecific antibodies of the invention or fragments thereof.

The terms “host cell” “host cell line” and “host cell culture” are used interchangeably and refer to cells into which exogenous nucleic acid has been introduced, including the progeny of such cells. Host cells include “transformants” and “transformed cells” which include the primary transformed cell and progeny derived therefrom without regard to the number of passages. Progeny may not be completely identical in nucleic acid content to a parent cell, but may contain mutations. Mutant progeny that have the same function or biological activity as screened or selected for in the originally transformed cell are included herein. A host cell is any type of cellular system that can be used to generate the antibodies or bispecific antibodies of the present invention. Host cells include cultured cells, e.g. mammalian cultured cells, such as HEK cells, CH0 cells, BHK cells, NS0 cells, SP2/0 cells, Y0 myeloma cells, P3X63 mouse myeloma cells, PER cells, PER.C6 cells or hybridoma cells, yeast cells, insect cells, and plant cells, to name only a few, but also cells comprised within a transgenic animal, transgenic plant or cultured plant or animal tissue. An “activating Fc receptor” is an Fc receptor that following engagement by an Fc domain of an antibody elicits signaling events that stimulate the receptor-bearing cell to perform effector functions. Human activating Fc receptors include FcγRIIIa (CD16a), FcγRI (CD64), FcγRIIa (CD32), and FcαRI (CD89).

Antibody-dependent cell-mediated cytotoxicity (ADCC) is an immune mechanism leading to the lysis of antibody-coated target cells by immune effector cells. The target cells are cells to which antibodies or derivatives thereof comprising an Fc region specifically bind, generally via the protein part that is N-terminal to the Fc region. As used herein, the term “reduced ADCC” is defined as either a reduction in the number of target cells that are lysed in a given time, at a given concentration of antibody in the medium surrounding the target cells, by the mechanism of ADCC defined above, and/or an increase in the concentration of antibody in the medium surrounding the target cells, required to achieve the lysis of a given number of target cells in a given time, by the mechanism of ADCC. The reduction in ADCC is relative to the ADCC mediated by the same antibody produced by the same type of host cells, using the same standard production, purification, formulation and storage methods (which are known to those skilled in the art), but that has not been engineered. For example the reduction in ADCC mediated by an antibody comprising in its Fc domain an amino acid substitution that reduces ADCC, is relative to the ADCC mediated by the same antibody without this amino acid substitution in the Fc domain. Suitable assays to measure ADCC are well known in the art (see e.g. PCT publication no. WO 2006/082515 or PCT publication no. WO 2012/130831).

An “effective amount” of an agent refers to the amount that is necessary to result in a physiological change in the cell or tissue to which it is administered.

A “therapeutically effective amount” of an agent, e.g. a pharmaceutical composition, refers to an amount effective, at dosages and for periods of time necessary, to achieve the desired therapeutic or prophylactic result. A therapeutically effective amount of an agent for example eliminates, decreases, delays, minimizes or prevents adverse effects of a disease.

An “individual”, “subject” or “patient” is a mammal. Mammals include, but are not limited to, domesticated animals (e.g. cows, sheep, cats, dogs, and horses), primates (e.g. humans and non-human primates such as monkeys), rabbits, and rodents (e.g. mice and rats). Particularly, the individual, subject or patient is a human.

The term “pharmaceutical composition” refers to a preparation which is in such form as to permit the biological activity of an active ingredient contained therein to be effective, and which contains no additional components which are unacceptably toxic to a subject to which the composition would be administered.

A “pharmaceutically acceptable carrier” refers to an ingredient in a pharmaceutical composition, other than an active ingredient, which is nontoxic to a subject. A pharmaceutically acceptable carrier includes, but is not limited to, a buffer, excipient, stabilizer, or preservative.

As used herein, “treatment” (and grammatical variations thereof such as “treat” or “treating”) refers to clinical intervention in an attempt to alter the natural course of a disease in the individual being treated, and can be performed either for prophylaxis or during the course of clinical pathology.

Desirable effects of treatment include, but are not limited to, preventing occurrence or recurrence of disease, alleviation of symptoms, diminishment of any direct or indirect pathological consequences of the disease, preventing metastasis, decreasing the rate of disease progression, amelioration or palliation of the disease state, and remission or improved prognosis. In some embodiments, antibodies or bispecific antibodies of the invention are used to delay development of a disease or to slow the progression of a disease.

The term “package insert” is used to refer to instructions customarily included in commercial packages of therapeutic products, that contain information about the indications, usage, dosage, administration, combination therapy, contraindications and/or warnings concerning the use of such therapeutic products.

Ant i-CLL-1 Antibody

An anti-CLL-1 antibody may comprise an anti-CLL-1 antibody or an antigen-binding fragment thereof as a CCL-1 targeting moiety. The anti-CCL-1 antibody or antigen-binding fragment thereof may exhibit potent binding and inhibitory activities to CCL-1, and be useful for therapeutic and diagnostics uses.

In one embodiment, the anti-CLL-1 antibody or fragment thereof may be capable of specificity to a human CLL-1 protein.

In one embodiment, the anti-CLL-1 antibody or fragment thereof may comprise (a) a VH CDR1 comprising an amino acid sequence selected from the group consisting of SEQ ID NOs: 1, 2 and 3; (b) a VH CDR2 comprising an amino acid sequence selected from the group consisting of SEQ ID NOs: 4, 5 and 6; (c) a VH CDR3 comprising an amino acid sequence selected from the group consisting of SEQ ID NOs: 7, 8, 9, 10 and 11; (d) a VL CDR1 comprising an amino acid sequence selected from the group consisting of SEQ ID NOs: 12, 13, 14 and 15; (e) a VL CDR2 comprising an amino acid sequence selected from the group consisting of SEQ ID NOs: 16, 17 and 18; and (f) a VL CDR3 comprising an amino acid sequence selected from the group consisting of SEQ ID NOs: 19, 20, 21 and 22.

The CDR sequences of anti-CLL-1 to be comprised in heavy chain and light chain variable regions of the antibody or antigen-binding fragment according to one embodiment of the present invention are shown in table 2 below.

TABLE 2

| | | SEQ ID |
|---|---|---|
| Name | Sequence | NO: |
| VH CDR1 | GYYMH | 1 |
| | GYAMN | 2 |
| | SYVIH | 3 |
| VH CDR2 | HVNPYNGATSFNRNFKD | 4 |
| | LINPYNGGAMYNQKFKG | 5 |
| | LFNPYNDDVNYNEKFKG | 6 |
| VH CDR3 | SADGYYERHFDY | 7 |
| | DYRYDGHLDY | 8 |
| | EGVHYGRPWFGY | 9 |
| | SAYGYYERHFDY | 10 |
| | DYRYEGHLDY | 11 |
| VL CDR1 | KASQSVDYDGDSYMN | 12 |
| | SATSSVSYMH | 13 |
| | KASQDIKSYLN | 14 |
| | KASQSVDYDADSYMN | 15 |
| VL CDR2 | AASNLQS | 16 |
| | DTSKLAS | 17 |
| | RASRLVD | 18 |
| VL CDR3 | QQSDKDPLT | 19 |
| | QQWSSDSPT | 20 |
| | LQYDEFPLT | 21 |
| | QQSDRDPLT | 22 |

In one embodiment, CDRs of each variable region of light chain and CDRs of each variable region of heavy chain disclosed in Table above(Table 2) can be combined freely.

In some embodiments, an antibody or fragment thereof may include no more than one, no more than two, or no more than three of substitutions.

In one embodiment, the anti-CLL-1 antibody or fragment thereof may comprise a heavy chain constant region comprising an amino acid sequence selected from the group consisting of SEQ ID NOs: 23 and 24; or a peptide having at least about 90%, 91%, 92%, 93%, 94%, 95%, 96%, 97%, 98% or 99% sequence identity to an amino acid sequence selected from the group consisting of SEQ ID NOs: 23 and 24.

In one embodiment, the anti-CLL-1 antibody or fragment thereof may comprise a light chain constant region comprising an amino acid sequence consisting of SEQ ID NO: 55.

In one embodiment, the anti-CLL-1 antibody or fragment thereof may comprise a heavy chain constant region comprising an amino acid sequence selected from the group consisting of SEQ ID NOs: 23 and 24; and a light chain constant region comprising an amino acid sequence consisting of SEQ ID NO: 55.

In one embodiment, the anti-CLL-1 antibody or fragment thereof may comprise a heavy chain variable region comprising an amino acid sequence selected from the group consisting of SEQ ID NOs: 25, 26, 27, 28, 29, 30, 31, 32, 33, 34, 35 and 74; or a peptide having at least about 90%, 91%, 92%, 93%, 94%, 95%, 96%, 97%, 98% or 99% sequence identity to an amino acid sequence selected from the group consisting of SEQ ID NOs: 25, 26, 27, 28, 29, 30, 31, 32, 33, 34, 35 and 74.

In one embodiment, the anti-CLL-1 antibody or fragment thereof may comprise a light chain variable region comprising an amino acid sequence selected from the group consisting of SEQ ID NOs: 36, 37, 38, 39, 40, 41, 42, 43, 44 and 75; or a peptide having at least about 90%, 91%, 92%, 93%, 94%, 95%, 96%, 97%, 98% or 99% sequence identity to an amino acid sequence selected from the group consisting of SEQ ID NOs: 36, 37, 38, 39, 40, 41, 42, 43, 44 and 75.

In one embodiment, the anti-CLL-1 antibody or fragment thereof may comprise a heavy chain variable region comprising an amino acid sequence selected from the group consisting of SEQ ID NOs: 25, 26, 27, 28, 29, 30, 31, 32, 33, 34, 35 and 74; and a light chain variable region comprising an amino acid sequence selected from the group consisting of SEQ ID NOs: 36, 37, 38, 39, 40, 41, 42, 43, 44 and 75.

In one embodiment, the anti-CLL-1 antibody or fragment thereof may comprise a heavy chain comprising an amino acid sequence consisting of SEQ ID NO: 45; or a peptide having at least about 90%, 91%, 92%, 93%, 94%, 95%, 96%, 97%, 98% or 99% sequence identity to an amino acid sequence consisting of SEQ ID NO: 45.

In one embodiment, the anti-CLL-1 antibody or fragment thereof may comprise a light chain comprising an amino acid sequence consisting of SEQ ID NO: 46; or a peptide having at least about 90%, 91%, 92%, 93%, 94%, 95%, 96%, 97%, 98% or 99% sequence identity to an amino acid sequence consisting of SEQ ID NO: 46.

In one embodiment, the anti-CLL-1 antibody or fragment thereof may comprise a heavy chain comprising an amino acid sequence consisting of SEQ ID NO: 45; and a light chain comprising an amino acid sequence consisting of SEQ ID NO: 46.

In one embodiment, the heavy chain constant region, heavy chain and light chain variable regions, the heavy chain and the light chain of the antibody or antigen-binding fragment may be exemplified in the following Table below(Table 3).

TABLE 3

| | SEQ |
|---|---|
| | ID |
| | NO |
| Heavy Chain Constant Region (CH) Sequence | |
| ASTKGPSVFPLAPSSKSTSGGTAALGCLVKDYFPEPVTVSWNSGALTSGVHTFPAVLQSSGLYSLSSVVTVPSSSLGTQT | 23 |
| YICNVNHKPSNTKVDKKVEPKSCDKTHTCPPCPAPELLGGPSVFLFPPKPKDTLMISRTPEVTCVVVDVSHEDPEVKFNW | |
| YVDGVEVHNAKTKPREEQYNSTYRVVSVLTVLHQDWLNGKEYKCKVSNKALPAPIEKTISKAKGQPREPQVYTLPPSREE | |
| MTKNQVSLTCLVKGFYPSDIAVEWESNGQPENNYKTTPPVLDSDGSFFLYSKLTVDKSRWQQGNVFSCSVMHEALHNHYT | |
| QKSLSLSPGK | |
| ASTKGPSVFPLAPSSKSTSGGTAALGCLVKDYFPEPVTVSWNSGALTSGVHTFPAVLQSSGLYSLSSVVTVPSSSLGTQT | 24 |
| YICNVNHKPSNTKVDKKVEPKSCDKTHTCPPCPAPELLGGPSVFLFPPKPKDTLMISRTPEVTCVVVDVSHEDPEVKFNW | |
| YVDGVEVHNAKTKPREEQYASTYRVVSVLTVLHQDWLNGKEYKCKVSNKALPAPIEKTISKAKGQPREPQVYTLPPSREE | |
| MTKNQVSLTCLVKGFYPSDIAVEWESNGQPENNYKTTPPVLDSDGSFFLYSKLTVDKSRWQQGNVFSCSVMHEALHNHYT | |
| QKSLSLSPGK | |
| Heavy Chain Variable Region (VH) Sequence | |
| EVQLQQSGPELVKPGASVKISCKASGYSFTGYYMHWVKQSHVKSLEWIGHVNPYNGATSFNRNFKDKASLTVDKSSSTAY | 25 |
| MELHSLTSEDSAVYYCGRSADGYYERHFDYWGQGTTLTVSS | |
| EVQLQQSGPELVKPGASMKISCKASGYSFTGYAMNWVKQSHGKNLEWIGLINPYNGGAMYNQKFKGKATLTVDKSTSTAY | 26 |
| MELLSLTSEDSAVYYCARDYRYDGHLDYWGQGTTLTVSS | |
| EVQLQQSGPELVKSGASVKMSCKASGYTFTSYVIHWVKQMPGQGLEWIGLFNPYNDDVNYNEKFKGKATLTSDKYSSTAY | 27 |
| LDLSSLTSEDSAVYYCAREGVHYGRPWFGYWGQGTLVTVSA | |
| EVQLQQSGPELVKPGASVKISCKASGYSFTGYYMHWVKQSHVKSLEWIGHVNPYNGATSFNRNFKDKASLTVDKSSSTAY | 28 |
| MELHSLTSEDSAVYYCGRSAYGYYERHFDYWGQGTTLTVSS | |
| EVQLQQSGPELVKPGASMKISCKASGYSFTGYAMNWVKQSHGKNLEWIGLINPYNGGAMYNQKFKGKATLTVDKSTSTAY | 29 |
| MELLSLTSEDSAVYYCARDYRYEGHLDYWGQGTTLTVSS | |
| QVQLVQSGAEVKKPGASVKVSCKASGYSFTGYYMHWVRQAPGQGLEWMGHVNPYNGATSFNRNFKDRVTMTRDTSTSTVY | 30 |
| MELSSLRSEDTAVYYCARSADGYYERHFDYWGQGTLVTVSS | |
| QVQLVQSGAEVKKPGASVKVSCKASGYSFTGYYMHWVRQAPGQGLEWIGHVNPYNGATSFNRNFKDRVTMTVDTSTSTVY | 31 |
| MELSSLRSEDTAVYYCGRSADGYYERHFDYWGQGTLVTVSS | |
| QVQLVQSGAEVKKPGASVKISCKASGYSFTGYYMHWVKQSHGQGLEWIGHVNPYNGATSFNRNFKDRASLTVDTSTSTVY | 32 |
| MELSSLRSEDSAVYYCGRSADGYYERHFDYWGQGTLVTVSS | |
| QVQLVQSGAEVKKPGASVKVSCKASGYSFTGYAMNWVRQAPGQRLEWIGLINPYNGGAMYNQKFKGRATLTVDTSASTAY | 33 |
| MELSSLRSEDTAVYYCARDYRYDGHLDYWGQGTLVTVSS | |
| QVQLVQSGAEVKKPGASVKVSCKASGYSFTGYAMNWVRQAPGQRLEWIGLINPYNGGAMYNQKFKGRATLTVDTSASTAY | 34 |
| MELSSLRSEDTAVYYCARDYRYEGHLDYWGQGTLVTVSS | |
| QVQLVQSGAEVKKPGASVKISCKASGYSFTGYAMNWVKQAPGQRLEWIGLINPYNGGAMYNQKFKGRATLTVDTSASTAY | 35 |
| MELSSLRSEDTAVYYCARDYRYDGHLDYWGQGTLVTVSS | |
| QVQLVQSGAEVKKPGASVKISCKASGYSFTGYYMHWVKQSHGQGLEWIGHVNPYNGATSFNRNFKDRASLTVDTSTSTVY | 74 |
| MELSSLRSEDSAVYYCGRSAYGYYERHFDYWGQGTLVTVSS | |
| Light Chain Variable Region (VL) Sequence | |
| DIVLTQSPASLAVSLGQRATISCKASQSVDYDGDSYMNWYQQKPGQPPKLLIYAASNLQSGIPARFSGSGSGTDFTLNIH | 36 |
| PVEEEDAATYYCQQSDKDPLTFGAGTKLELK | |
| QIVLTQSPVIMSASPGEKVTMTCSATSSVSYMHWYQQKSGTSPKRWIYDTSKLASGVPVRFSGSGSGTSYSLTISRMEAE | 37 |
| DAATYYCQQWSSDSPTFGVGTKLELK | |
| DIKMTQSPSSMNASLGERVTITCKASQDIKSYLNWFQQKPGKSPKTLIYRASRLVDGVPARFSGSESGQDYSLTISSLEF | 38 |
| EDMGIYYCLQYDEFPLTFGAGTKLELK | |
| DIVLTQSPASLAVSLGQRATISCKASQSVDYDADSYMNWYQQKPGQPPKLLIYAASNLQSGIPARFSGSGSGTDFTLNIH | 39 |
| PVEEEDAATYYCQQSDRDPLTFGAGTKLELK | |
| DIKMTQSPSSMsASLGERVTITCKASQDIKSYLNWFQQKPGKSPKTLIYRASRLVDGVPARFSGSESGQDYSLTISSLEF | 40 |
| EDMGIYYCLQYDEFPLTFGAGTKLELK | |
| DIVMTQSPDSLAVSLGERATINCKASQSVDYDGDSYMNWYQQKPGQPPKLLIYAASNLQSGVPDRFSGSGSGTDFTLTIS | 41 |
| SLQAEDVAVYYCQQSDKDPLTFGGGTKLEIK | |
| EIVLTQSPATLSLSPGERATLSCSATSSVSYMHWYQQKPGQAPRRWIYDTSKLASGIPARFSGSGSGTSYTLTISSLEPE | 42 |
| DFAVYYCQQWSSDSPTFGQGTKLEIK | |
| EIVLTQSPATLSLSPGERATMSCSATSSVSYMHWYQQKPGTAPRRWIYDTSKLASGIPARFSGSGSGTDYTLTISSLEPE | 43 |
| DFAVYYCQQWSSDSPTFGQGTKLEIK | |
| EIVLTQSPATLSLSPGERATLSCSATSSVSYMHWYQQKSGTAPRRWIYDTSKLASGIPARFSGSGSGTDYTLTISSLEPE | 44 |
| DAAVYYCQQWSSDSPTFGQGTKLEIK | |
| DIVMTQSPDSLAVSLGERATINCKASQSVDYDADSYMNWYQQKPGQPPKLLIYAASNLQSGVPDRFSGSGSGTDFTLTIS | 75 |
| SLQAEDVAVYYCQQSDRDPLTFGGGTKLEIK | |
| Heavy Chain | |
| QVQLVQSGAEVKKPGASVKVSCKASGYSFTGYAMNWVRQAPGQRLEWIGLINPYNGGAMYNQKFKGRATLTVDTSASTAY | 45 |
| MELSSLRSEDTAVYYCARDYRYEGHLDYWGQGTLVTVSSASTKGPSVFPLAPSSKSTSGGTAALGCLVKDYFPEPVTVSW | |
| NSGALTSGVHTFPAVLQSSGLYSLSSVVTVPSSSLGTQTYICNVNHKPSNTKVDKKVEPKSC | |
| Light Chain | |
| EIVLTQSPATLSLSPGERATLSCSATSSVSYMHWYQQKPGQAPRRWIYDTSKLASGIPARFSGSGSGTSYTLTISSLEPE | 46 |
| DFAVYYCQQWSSDSPTFGQGTKLEIKRTVAAPSVFIFPPSDEQLKSGTASVVCLLNNFYPREAKVQWKVDNALQSGNSQE | |
| SVTEQDSKDSTYSLSSTLTLSKADYEKHKVYACEVTHQGLSSPVTKSFNRGEC | |

In other embodiment, the variable regions of heavy chain and light chain disclosed in Table above(Table 3) can be combined freely for preparation of various forms of antibodies.

Each of heavy chain and light chain variable regions disclosed herein may bind to targeting various heavy chain and light chain constant regions to form heavy chain and light chain of an intact antibody, respectively. In addition, each of heavy chain and light chain sequences bound to constant regions like this may be also combined to form an intact antibody structure.

Any variable region of heavy chain and light chain of the antibody may be linked to at least a part of constant regions. The constant regions may be selected according to whether antibody-dependent cell-mediated cytotoxicity, antibody-dependent cell phagocytosis and/or complement-dependent cytotoxicity, etc. is required. For example, Human isotype IgG1 and IgG3 have complement-dependent cytotoxicity, and human isotype IgG2 and IgG4 do not have the cytotoxicity. Human IgG1 and IgG3 also induce a cell-mediated effector function stronger than human IgG2 and IgG4. For example, the heavy chain variable region may bind to a constant region of IgG, such as IgG1, IgG2, IgG2a, IgG2b, IgG3 and IgG4, and the light chain variable region may bind to a kappa or lambda constant region. For the constant region, one appropriate as desired can be used, and for example, a human or mouse-derived one can be used. In one embodiment, a human heavy chain constant region IgG1 is used. In other embodiment, as the light chain constant region, a human lambda region may be used.

Any variable region disclosed herein may be bound to a constant region, thereby forming heavy chain and light chain sequences. In one embodiment, the heavy chain variable region disclosed herein may be bound to a human IgG1 constant region, to form a heavy chain (full-length). In other embodiment, the light chain variable region disclosed herein may be bound to a human lambda constant region, to form and the light chain (full-length). The light chain and heavy chain can be combined as various combinations, thereby forming an intact antibody consisting of two light chains and two heavy chains.

In other embodiment, the antibody may comprise or consist essentially of a combination of a heavy chain and a light chain, which are represented by the following sequence: SEQ ID NOs: 45; and SEQ ID NOs: 46.

However, such constant region sequences to be combined with the variable regions disclosed herein are exemplary, and those skilled in the art will know that other constant regions including IgG1 heavy chain constant region, IgG3 or IgG4 heavy chain constant region, any kappa or lambda light chain constant region, constant regions modified for stability, expression, manufacturability or other targeting properties, etc. may be used.

In some embodiments, the antigen-binding fragment of the anti-CLL-1 antibody may be any fragment comprising heavy chain CDRs and/or light chain CDRs of the antibody, and for example, it may be selected from, but not limited to, the group consisting of Fab, Fab′, F(ab′)₂, xFab, Fd (comprising a heavy chain variable region and a CH1 domain), Fv (a heavy chain variable region and/or a light chain variable region), single-chain Fv (scFv; comprising or consisting essentially of a heavy chain variable region and a light chain variable region, in any order, and a peptide linker between the heavy chain variable region and the light chain variable region), single-chain antibodies, disulfide-linked Fvs (sdFv), scFab (single chain Fab), scFab-Fc (comprising scFab and Fc region), half-IgG (comprising one light chain and one heavy chain) and the like.

The present invention may comprise one or more amino acid sequences having substantial sequence identity with one or more amino acid sequences disclosed herein. The substantial identity means maintaining the effect disclosed herein in which the sequence variation is present.

In some embodiments, the anti-CLL-1 antibody or antigen-binding fragment thereof may be a mouse antibody, a chimeric antibody, a humanized antibody, or a fully human antibody.

In one embodiment, the anti-CLL-1 antibody or antigen-binding fragment thereof may be capable of fuse with polypeptide(s) to form a fusion protein. The polypeptide(s) may be an antibody or antigen. In one embodiment, the fusion protein may be in the form of multispecific antibody (e.g., bispecific antibody).

In one embodiment, the disclosure provides fusion proteins comprising (a) one or more single domain antibodies, or antigen-binding fragments thereof, described herein (e.g., one or more CDRs described herein), and (b) one or more additional polypeptides. For example, a fusion protein can include one or more single domain antibodies described herein and a constant region or Fc region described herein. In one embodiment, one or more single domain antibodies, or antigen-binding fragments thereof, described herein (e.g., one or more CDRs described herein) can be conjugated noncovalently or covalently, e.g., fused, to an antibody or antigen.

### Anti-CD3 Antibody

An anti-CD3 antibody may comprise an anti-CD3 antibody or an antigen-binding fragment thereof as a CD3 targeting moiety. The anti-CD3 antibody or antigen-binding fragment thereof may exhibit potent binding and inhibitory activities to CD3, and be useful for therapeutic and diagnostics uses.

In one embodiment, the anti-CD3 antibody or fragment thereof may be capable of specificity to a human CD3 protein, preferably human CD3E polypeptide.

In one embodiment, the anti-CD3 antibody or fragment thereof may comprise (a) a VH CDR1 comprising an amino acid sequence consisting of SEQ ID NO:47; (b) a VH CDR2 comprising an amino acid sequence consisting of SEQ ID NO: 48; (c) VH CDR3 comprising an amino acid sequence consisting of SEQ ID NO: 49; (d) a VL CDR1 comprising an amino acid sequence consisting of SEQ ID NO: 50; (e) a VL CDR2 comprising an amino acid sequence consisting of SEQ ID NO: 51; and (f) a VL CDR3 comprising an amino acid sequence consisting of SEQ ID NO: 52.

In one embodiment, CDRs of each variable region of light chain and CDRs of each variable region of heavy chain disclosed above can be combined freely.

In some embodiments, an antibody or fragment thereof may include no more than one, no more than two, or no more than three of substitutions.

In one embodiment, the anti-CD3 antibody or fragment thereof may comprise a heavy chain comprising an amino acid sequence consisting of SEQ ID NO: 53 or a peptide having at least about 90%, 91%, 92%, 93%, 94%, 95%, 96%, 97%, 98% or 99% sequence identity to an amino acid sequence consisting of SEQ ID NO: 53; and a light chain comprising an amino acid sequence consisting of SEQ ID NO: 54 or a peptide having at least about 90%, 91%, 92%, 93%, 94%, 95%, 96%, 97%, 98% or 99% sequence identity to an amino acid sequence consisting of SEQ ID NO: 54.

In other embodiment, the variable regions of heavy chain and light chain disclosed above can be combined freely for preparation of various forms of antibodies.

Each of heavy chain and light chain variable regions disclosed herein may bind to targeting various heavy chain and light chain constant regions to form heavy chain and light chain of an intact antibody, respectively. In addition, each of heavy chain and light chain sequences bound to constant regions like this may be also combined to form an intact antibody structure.

Any variable region of heavy chain and light chain of the antibody may be linked to at least a part of constant regions. The constant regions may be selected according to whether antibody-dependent cell-mediated cytotoxicity, antibody-dependent cell phagocytosis and/or complement-dependent cytotoxicity, etc. is required. For example, Human isotype IgG1 and IgG3 have complement-dependent cytotoxicity, and human isotype IgG2 and IgG4 do not have the cytotoxicity. Human IgG1 and IgG3 also induce a cell-mediated effector function stronger than human IgG2 and IgG4. For example, the heavy chain variable region may bind to a constant region of IgG, such as IgG1, IgG2, IgG2a, IgG2b, IgG3 and IgG4, and the light chain variable region may bind to a kappa or lambda constant region. For the constant region, one appropriate as desired can be used, and for example, a human or mouse-derived one can be used. In one embodiment, a human heavy chain constant region IgG1 is used. In other embodiment, as the light chain constant region, a human lambda region may be used.

Any variable region disclosed herein may be bound to a constant region, thereby forming heavy chain and light chain sequences. In one embodiment, the heavy chain variable region disclosed herein may be bound to a human IgG1 constant region, to form a heavy chain (full-length). In other embodiment, the light chain variable region disclosed herein may be bound to a human lambda constant region, to form and the light chain (full-length). The light chain and heavy chain can be combined as various combinations, thereby forming an intact antibody consisting of two light chains and two heavy chains.

In other embodiment, the antibody may comprise or consist essentially of a combination of a heavy chain and a light chain, which are represented by the following sequence: SEQ ID NO: 53; and SEQ ID NO: 54.

However, such constant region sequences to be combined with the variable regions disclosed herein are exemplary, and those skilled in the art will know that other constant regions including IgG1 heavy chain constant region, IgG3 or IgG4 heavy chain constant region, any kappa or lambda light chain constant region, constant regions modified for stability, expression, manufacturability or other targeting properties, etc. may be used.

In some embodiments, the antigen-binding fragment of the anti-CD3 antibody may be any fragment comprising heavy chain CDRs and/or light chain CDRs of the antibody, and for example, it may be selected from, but not limited to, the group consisting of Fab, Fab′, F(ab′)₂, xFab, Fd (comprising a heavy chain variable region and a CH1 domain), Fv (a heavy chain variable region and/or a light chain variable region), single-chain Fv (scFv; comprising or consisting essentially of a heavy chain variable region and a light chain variable region, in any order, and a peptide linker between the heavy chain variable region and the light chain variable region), single-chain antibodies, disulfide-linked Fvs (sdFv), scFab (single chain Fab), scFab-Fc (comprising scFab and Fc region), half-IgG (comprising one light chain and one heavy chain) and the like.

The present invention comprises one or more amino acid sequences having substantial sequence identity with one or more amino acid sequences disclosed herein. The substantial identity means maintaining the effect disclosed herein in which the sequence variation is present.

In one embodiment, the disclosure provides fusion proteins comprising (i) one or more single domain antibodies, or antigen-binding fragments thereof, described herein (e.g., one or more CDRs described herein), and (ii) one or more additional polypeptides. For example, a fusion protein can include one or more single domain antibodies described herein and a constant region or Fc region described herein. In one embodiment, one or more single domain antibodies, or antigen-binding fragments thereof, described herein (e.g., one or more CDRs described herein) can be conjugated noncovalently or covalently, e.g., fused, to an antibody or antigen.

### Anti-CLL-1/Anti-CD3 Bispecific Antibody

An anti-CLL-1 antibody/anti-CD3 bispecific antibody may comprise the anti-CLL-1 antibody or antigen-binding fragment thereof; and anti-CD3 antibody or antigen-binding fragment thereof. The anti-CLL-1 antibody/anti-CD3 bispecific antibody may be useful for therapeutic and diagnostics uses.

In one embodiment, in the bispecific antibody comprising the CLL-1 targeting moiety and the CD3 targeting moiety.

In one embodiment, the anti-CLL-1 antibody or antigen-binding fragment thereof and the anti-CD3 antibody or antigen-binding fragment thereof may be fused to each other, directly or via a peptide linker.

In one embodiment, each of the anti-CLL-1 antibody or antigen-binding fragment thereof and the anti-CD3 antibody or antigen-binding fragment thereof may be independently a Fab molecule.

In one embodiment, said Fab molecule may be a human Fab molecule or a chimeric Fab-like domain containing a TCR constant region. In one embodiment, said Fab molecule may be chimeric or humanized.

In one embodiment, (a) the anti-CLL-1 antibody or antigen-binding fragment thereof may be fused, at the C-terminus of the Fab heavy chain of the anti-CLL-1 antibody or antigen-binding fragment thereof, to the N-terminus of the Fab heavy chain of the anti-CD3 antibody or antigen-binding fragment thereof, or (b) the anti-CD3 antibody or antigen-binding fragment thereof may be fused, at the C-terminus of the Fab heavy chain of the anti-CD3 antibody or antigen-binding fragment thereof, to the N-terminus of the Fab heavy chain of the anti-CLL-1 antibody or antigen-binding fragment thereof.

In one embodiment, the anti-CLL-1/anti-CD3 bispecific antibody may comprise an Fc domain comprising a first sub-unit and a second sub-unit.

In one embodiment, the Fc domain may be a human Fc domain with or without additional mutations. Such additional mutations in a Fc domain may include N297A mutation to null ADCC activity or KIH (knob-into-hole) mutation to promote the correct antibody pairing, but it may not be limited thereto.

In one embodiment, the anti-CLL-1 antibody or antigen-binding fragment thereof and the anti-CD3 antibody or antigen-binding fragment thereof may be each a Fab molecule; and (a) the anti-CD3 antibody or antigen-binding fragment thereof may be fused, at the C-terminus of a Fab heavy chain of the anti-CD3 antibody or antigen-binding fragment thereof, to the N-terminus of the first sub-unit of the Fc domain, and (b) the anti-CLL-1 antibody or antigen-binding fragment thereof may be fused, at the C-terminus of a Fab heavy chain of the anti-CLL-1 antibody or antigen-binding fragment thereof, to the N-terminus of the second sub-unit of the Fc domain.

In one embodiment, the anti-CLL-1 antibody or antigen-binding fragment thereof may be a first anti-CLL-1 antibody or an antigen-binding fragment thereof, and further comprise a second anti-CLL-1 antibody or an antigen-binding fragment thereof.

In one embodiment, the first anti-CLL-1 antibody or antigen-binding fragment thereof, the anti-CD3 antibody or antigen-binding fragment thereof and, where present, the second anti-CLL-1 antibody or antigen-binding fragment thereof may be each a Fab molecule; either (a) the ant i-CD3 antibody or ant igen-binding fragment thereof may be fused, at the C-terminus of the Fab heavy chain of the anti-CD3 antibody or antigen-binding fragment thereof, to the N-terminus of the Fab heavy chain of the first anti-CLL-1 antibody or antigen-binding fragment thereof and the first anti-CLL-1 antibody or antigen-binding fragment thereof may be fused, at the C-terminus of the Fab heavy chain of the first anti-CLL-1 antibody or antigen-binding fragment thereof, to the N-terminus of the first sub-unit of the Fc domain, or (b) the first anti-CLL-1 antibody or antigen-binding fragment thereof may be fused, at the C-terminus of the Fab heavy chain of the first anti-CLL-1 antibody or antigen-binding fragment thereof, to the N-terminus of the Fab heavy chain of the anti-CD3 antibody or antigen-binding fragment thereof and the anti-CD3 antibody or antigen-binding fragment thereof may be fused, at the C-terminus of the Fab heavy chain of the anti-CD3 antibody or antigen-binding fragment thereof, to the N-terminus of the first sub-unit of the Fc domain; and the second anti-CLL-1 antibody or antigen-binding fragment thereof, where present, may be fused, at the C-terminus of the Fab heavy chain of the second anti-CLL-1 antibody or antigen-binding fragment thereof, to the N-terminus of the second sub-unit of the Fc domain.

In one embodiment, the first anti-CLL-1 antibody or antigen-binding fragment thereof, the anti-CD3 antibody or antigen-binding fragment thereof and the second anti-CLL-1 antibody or antigen-binding fragment thereof are each a Fab molecule; the first anti-CLL-1 antibody or antigen-binding fragment thereof is fused, at the C-terminus of the Fab heavy chain of the first anti-CLL-1 antibody or antigen-binding fragment thereof, to the N-terminus of the Fab heavy chain of the anti-CD3 antibody or antigen-binding fragment thereof and the anti-CD3 antibody or antigen-binding fragment thereof is fused, at the C-terminus of the Fab heavy chain of the anti-CD3 antibody or antigen-binding fragment thereof, to the N-terminus of the first sub-unit of the Fc domain; and the second anti-CLL-1 antibody or antigen-binding fragment thereof, where present, is fused, at the C-terminus of the Fab heavy chain of the second anti-CLL-1 antibody or antigen-binding fragment thereof, to the N-terminus of the second sub-unit of the Fc domain. Due to the masked CD3 binding site in this structure, the bispecific antibody can strongly induce T cell activation and cytokine expression of IFN-g and IL-2 but weakly induce CRS-related cytokines such as TNF-a and IL-6, thus shows less off-tumor toxicity.

In one embodiment, the first anti-CLL-1 antibody or antigen-binding fragment thereof, and the second anti-CLL-1 antibody or an antigen-binding fragment thereof may be identical to each other.

In one embodiment, the bispecific antibody may be capable of simultaneous binding to CLL-1 and CD3 which is an activating T cell antigen. In one embodiment, the bispecific antibody may be capable of crosslinking a T cell and a target cell by simultaneous binding the CLL-1 and CD3. In one embodiment, such simultaneous binding results in lysis of the target cell, particularly a CLL-1 expressing tumor cell. In one embodiment, such simultaneous binding results in activation of the T cell. In other embodiments, such simultaneous binding results in a cellular response of a T lymphocyte, particularly a cytotoxic T lymphocyte, selected from the group of: proliferation, differentiation, cytokine secretion, cytotoxic effector molecule release, cytotoxic activity, and expression of activation markers.

In one embodiment, the bispecific antibody may be capable of re-directing cytotoxic activity of a T cell to a target cell. In a particular embodiment, said re-direction is independent of MHC-mediated peptide antigen presentation by the target cell and and/or specificity of the T cell. Particularly, a T cell according to any of the embodiments of the invention is a cytotoxic T cell. In some embodiments the T cell is a CD4⁺ or a CD8⁺ T cell, particularly a CD8⁺ T cell.

In one embodiment, the bispecific antibodies according to an embodiment may induce cytokine expression, granzyme B and/or perforin in the presence of U937 and HL-60 cell lines.

### Conjugates

The invention provides immunoconjugates comprising an anti-CLL-1 antibody or anti-CLL-1/anti-CD3 bispecific antibody as described herein conjugated (chemically bonded) to one or more therapeutic agents such as cytotoxic agents, chemotherapeutic agents, drugs, growth inhibitory agents, toxins (e.g., protein toxins, enzymatically active toxins of bacterial, fungal, plant, or animal origin, or fragments thereof), or radioactive isotopes.

In one embodiment, an immunoconjugate is an antibody-drug conjugate (ADC) in which an antibody is conjugated to one or more of the therapeutic agents mentioned above. The antibody is typically connected to one or more of the therapeutic agents using linkers. An overview of ADC technology including examples of therapeutic agents and drugs and linkers is set forth in Pharmacol Review 68:3-19 (2016).

In another embodiment, an immunoconjugate may comprise an antibody as described herein conjugated to an enzymatically active toxin or fragment thereof, including but not limited to diphtheria A chain, nonbinding active fragments of diphtheria toxin, exotoxin A chain (from Pseudomonas aeruginosa), ricin A chain, abrin A chain, modeccin A chain, alpha-sarcin, Aleurites fordii proteins, dianthin proteins, Phytolaca americana proteins (PAPI, PAPII, and PAP-S), Momordica charantia inhibitor, curcin, crotin, Sapaonaria officinalis inhibitor, gelonin, mitogellin, restrictocin, phenomycin, enomycin, and the tricothecenes.

In another embodiment, an immunoconjugate may comprise an antibody as described herein conjugated to a radioactive atom to form a radioconjugate. A variety of radioactive isotopes are available for the production of radioconjugates. Examples include At²¹¹, I¹³¹, I¹²⁵, Y⁹⁰, Re¹⁸⁶, Re¹⁸⁸, Sm⁵³, Bi²¹², P³², Pb²¹² and radioactive isotopes of Lu. When the radioconjugate is used for detection, it may comprise a radioactive atom for scintigraphic studies, for example tc99m or I123, or a spin label for nuclear magnetic resonance (NMR) imaging (also known as magnetic resonance imaging, mri), such as iodine-123 again, iodine-131, indium-111, fluorine-19, carbon-13, nitrogen-15, oxygen-17, gadolinium, manganese or iron.

Conjugates of an antibody and cytotoxic agent may be made using a variety of bifunctional protein coupling agents such as N-succinimidyl-3-(2-pyridyldithio) propionate (SPDP), succinimidyl-4-(N-maleimidomethyl) cyclohexane-1-carboxylate (SMCC), iminothiolane (IT), bifunctional derivatives of imidoesters (such as dimethyl adipimidate HCl), active esters (such as disuccinimidyl suberate), aldehydes (such as glutaraldehyde), bis-azido compounds (such as bis (p-azidobenzoyl) hexanediamine), bis-diazonium derivatives (such as bis-(p-diazoniumbenzoyl)-ethylenediamine), diisocyanates (such as toluene 2,6-diisocyanate), and bis-active fluorine compounds (such as 1,5-difluoro-2,4-dinitrobenzene). For example, a ricin immunotoxin can be prepared as described in Vitetta et al., Science 238:1098 (1987). Carbon-14-labeled 1-isothiocyanatobenzyl-3-methyldiethylene triaminepentaacetic acid (MX-DTPA) is an exemplary chelating agent for conjugation of radionucleotide to the antibody. See WO94/11026. The linker may be a “cleavable linker” facilitating release of a cytotoxic drug in the cell. For example, an acid-labile linker, peptidase-sensitive linker, photolabile linker, dimethyl linker or disulfide-containing linker (Chari et al., Cancer Res. 52:127-131 (1992); U.S. Pat. No. 5,208,020) may be used.

The immunuoconjugates or ADCs herein expressly contemplate, but are not limited to such conjugates prepared with cross-linker reagents including, but not limited to, BMPS, EMCS, GMBS, HBVS, LC-SMCC, MBS, MPBH, SBAP, SIA, SIAB, SMCC, SMPB, SMPH, sulfo-EMCS, sulfo-GMBS, sulfo-KMUS, sulfo-MBS, sulfo-SIAB, sulfo-SMCC, and sulfo-SMPB, and SVSB (succinimidyl-(4-vinylsulfone)benzoate) which are commercially available (e.g., from Pierce Biotechnology, Inc., Rockford, Ill., U.S.A).

Cytotoxic agent that can be conjugated also include, for example, pyrrolobenzodiazepine (PBD), Monomethyl Auristatin E (MMAE), Monomethyl Auristatin F (MMAF), camptothecin, doxorubicin, cisplatin, verapamil, fluorouracil, oxaliplatin, daunorubicin, irinotecan, topotecan, paclitaxel, carboplatin, gemcitabine, methotrexalte, docetaxel, acivicin, aclarubicin, acodazole, acronycine, adozelesin, alanosine, aldesleukin, allopurinol sodium, altretamine, aminoglutethimide, amonafide, ampligen, amsacrine, androgens, anguidine, aphidicolin glycinate, asaley, asparaginase, 5-azacitidine, azathioprine, Bacillus Calmette-Guerin (BCG), Baker's Antifol, beta-2-deoxythioguanosine, bisantrene HCl, bleomycin sulfate, busulfan, buthionine sulfoximine, BWA 773U82, BW 502U83/HCl, BW 7U85 mesylate, ceracemide, carbetimer, carboplatin, carmustine, chlorambucil, chloroquinoxaline-sulfonamide, chlorozotocin, chromomycin A3, cisplatin, cladribine, corticosteroids, Corynebacterium parvum, CPT-11, crisnatol, cyclocytidine, cyclophosphamide, cytarabine, cytembena, dabis maleate, dacarbazine, dactinomycin, daunorubicin HCl, deazauridine, dexrazoxane, dianhydrogalactitol, diaziquone, dibromodulcitol, didemnin B, diethyldithiocarbamate, diglycoaldehyde, dihydro-5-azacytidine, echinomycin, edatrexate, edelfosine, eflomithine, Elliott's solution, elsamitrucin, epirubicin, esorubicin, estramustine phosphate, estrogens, etanidazole, ethiofos, etoposide, fadrazole, fazarabine, fenretinide, filgrastim, finasteride, flavone acetic acid, floxuridine, fludarabine phosphate, 5-fluorouracil, Fluosol™, flutamide, gallium nitrate, gemcitabine, goserelin acetate, hepsulfam, hexamethylene bisacetamide, homoharringtonine, hydrazine sulfate, 4-hydroxyandrostenedione, hydrozyurea, idarubicin HCl, ifosfamide, interferon alpha, interferon beta, interferon gamma, interleukin-1 alpha and beta, interleukin-3, interleukin-4, interleukin-6, 4-ipomeanol, iproplatin, isotretinoin, leucovorin calcium, leuprolide acetate, levamisole, liposomal daunorubicin, liposome encapsulated doxorubicin, lomustine, lonidamine, maytansine, mechlorethamine hydrochloride, melphalan, menogaril, merbarone, 6-mercaptopurine, mesna, a methanol extract of Bacillus calmette-guerin, methotrexate, N-methylformamide, mifepristone, mitoguazone, mitomycin-C, mitotane, mitoxantrone hydrochloride, monocyte/macrophage colony-stimulating factor, nabilone, nafoxidine, neocarzinostatin, octreotide acetate, ormaplatin, oxaliplatin, paclitaxel, pala, pentostatin, piperazinedione, pipobroman, pirarubicin, piritrexim, piroxantrone hydrochloride, PIXY-321, plicamycin, porfimer sodium, prednimustine, procarbazine, progestins, pyrazofurin, razoxane, sargramostim, semustine, spirogermanium, spiromustine, streptonigrin, streptozocin, sulofenur, suramin sodium, tamoxifen, taxotere, tegafur, teniposide, terephthalamidine, teroxirone, thioguanine, thiotepa, thymidine injection, tiazofurin, topotecan, toremifene, tretinoin, trifluoperazine hydrochloride, trifluridine, trimetrexate, tumor necrosis factor (TNF), uracil mustard, vinblastine sulfate, vincristine sulfate, vindesine, vinorelbine, vinzolidine, Yoshi 864, zorubicin, pharmaceutically acceptable salts thereof, and mixtures thereof, etc.

### Glycosylation Variants

In certain embodiments, an antibody provided herein may be altered to increase or decrease the extent to which the antibody is glycosylated. Addition or deletion of glycosylation sites to an antibody may be conveniently accomplished by altering the amino acid sequence such that one or more glycosylation sites is created or removed.

Where the antibody comprises an Fc region, the oligosaccharide attached thereto may be altered. Native antibodies produced by mammalian cells typically comprise a branched, biantennary oligosaccharide that is generally attached by an N-linkage to Asn297 of the CH2 domain of the Fc region. See, e.g., Wright et al. TIBTECH 15:26-32 (1997). The oligosaccharide may include various carbohydrates, e.g., mannose, N-acetyl glucosamine (GlcNAc), galactose, and sialic acid, as well as a fucose attached to a GlcNAc in the “stem” of the biantennary oligosaccharide structure. In some embodiments, modifications of the oligosaccharide in an antibody of the invention may be made in order to create antibody variants with certain improved properties.

In one embodiment, antibody variants are provided having a non-fucosylated oligosaccharide, i.e. an oligosaccharide structure that lacks fucose attached (directly or indirectly) to an Fc region. Such non-fucosylated oligosaccharide (also referred to as “afucosylated” oligosaccharide) particularly is an N-linked oligosaccharide which lacks a fucose residue attached to the first GlcNAc in the stem of the biantennary oligosaccharide structure. In one embodiment, antibody variants are provided having an increased proportion of non-fucosylated oligosaccharides in the Fc region as compared to a native or parent antibody. For example, the proportion of non-fucosylated oligosaccharides may be at least about 20%, at least about 40%, at least about 60%, at least about 80%, or even about 100% (i.e. no fucosylated oligosaccharides are present). The percentage of non-fucosylated oligosaccharides is the (average) amount of oligosaccharides lacking fucose residues, relative to the sum of all oligosaccharides attached to Asn 297 (e. g. complex, hybrid and high mannose structures) as measured by MALDI-TOF mass spectrometry, as described in WO 2006/082515, for example. Asn297 refers to the asparagine residue located at about position 297 in the Fc region (EU numbering of Fc region residues); however, Asn297 may also be located about ±3 amino acids upstream or downstream of position 297, i.e., between positions 294 and 300, due to minor sequence variations in antibodies. Such antibodies having an increased proportion of non-fucosylated oligosaccharides in the Fc region may have improved FcγRIIIa receptor binding and/or improved effector function, in particular improved ADCC function. See, e.g., US 2003/0157108; US 2004/0093621.

Examples of cell lines capable of producing antibodies with reduced fucosylation include Lec13 CH0 cells deficient in protein fucosylation (Ripka et al. Arch. Biochem. Biophys. 249:533-545 (1986); US 2003/0157108; and WO 2004/056312, especially at Example 11), and knockout cell lines, such as alpha-1,6-fucosyltransferase gene, FUT8, knockout CH0 cells (see, e.g., Yamane-Ohnuki et al. Biotech. Bioeng. 87:614-622 (2004); Kanda, Y. et al., Biotechnol. Bioeng., 94(4):680-688 (2006); and WO2003/085107), or cells with reduced or abolished activity of a GDP-fucose synthesis or transporter protein (see, e.g., US2004259150, US2005031613, US2004132140, US2004110282).

In a further embodiment, antibody variants are provided with bisected oligosaccharides, e.g., in which a biantennary oligosaccharide attached to the Fc region of the antibody is bisected by GlcNAc. Such antibody variants may have reduced fucosylation and/or improved ADCC function as described above. Examples of such antibody variants are described, e.g., in Umana et al., Nat Biotechnol 17, 176-180 (1999); Ferrara et al., Biotechn Bioeng 93, 851-861 (2006); WO 99/54342; WO 2004/065540, WO 2003/011878.

Antibody variants with at least one galactose residue in the oligosaccharide attached to the Fc region are also provided. Such antibody variants may have improved CDC function. Such antibody variants are described, e.g., in WO 1997/30087; WO 1998/58964; and WO 1999/22764.

### Fc Domain

In particular embodiments, the antibody or bispecific antibody of the invention may comprise an Fc domain composed of a first sub-unit and a second sub-unit. It is understood, that the features of the Fc domain described herein in relation to the antibody or bispecific antibody can equally apply to an Fc domain comprised in an antibody of the invention.

The Fc domain of the antibody or bispecific antibody may consist of a pair of polypeptide chains comprising heavy chain domains of an immunoglobulin molecule. For example, the Fc domain of an immunoglobulin G (IgG) molecule is a dimer, each sub-unit of which comprises the CH2 and CH3 IgG heavy chain constant domains. The two sub-units of the Fc domain are capable of stable association with each other. In one embodiment, the antibody or bispecific antibody of the invention comprises not more than one Fc domain.

In one embodiment, the Fc domain of the antibody or bispecific antibody may be an IgG Fc domain. In a further particular embodiment, the Fc domain may be a human Fc domain.

### Fc Domain Modifications Promoting Heterodimerization

Antibodies or bispecific antibodies according to the invention may comprise different antigen binding moieties, which may be fused to one or the other of the two sub-units of the Fc domain, thus the two sub-units of the Fc domain are typically comprised in two non-identical polypeptide chains. Recombinant co-expression of these polypeptides and subsequent dimerization leads to several possible combinations of the two polypeptides. To improve the yield and purity of antibodies or bispecific antibodies in recombinant production, it will thus be advantageous to introduce in the Fc domain of the antibody or bispecific antibody a modification promoting the association of the desired polypeptides.

Accordingly, in particular embodiments, the Fc domain of the antibody or bispecific antibody according to the invention may comprise a modification promoting the association of the first and the second sub-unit of the Fc domain. The site of most extensive protein-protein interaction between the two sub-units of a human IgG Fc domain is in the CH3 domain of the Fc domain. Thus, in one embodiment said modification may be in the CH3 domain of the Fc domain.

There exist several approaches for modifications in the CH3 domain of the Fc domain in order to enforce heterodimerization, which are well described e.g. in WO 96/27011, WO 98/050431, EP 1870459, WO 2007/110205, WO 2007/147901, WO 2009/089004, WO 2010/129304, WO 2011/90754, WO 2011/143545, WO 2012058768, WO 2013157954, WO 2013096291. Typically, in all such approaches the CH3 domain of the first sub-unit of the Fc domain and the CH3 domain of the second sub-unit of the Fc domain are both engineered in a complementary manner so that each CH3 domain (or the heavy chain comprising it) can no longer homodimerize with itself but is forced to heterodimerize with the complementarily engineered other CH3 domain (so that the first and second CH3 domain heterodimerize and no homodimers between the two first or the two second CH3 domains are formed). These different approaches for improved heavy chain heterodimerization are contemplated as different alternatives in combination with the heavy-light chain modifications (e.g. VH and VL exchange/replacement in one binding arm and the introduction of substitutions of charged amino acids with opposite charges in the CH1/CL interface) in the antibody or bispecific antibody which reduce heavy/light chain mispairing and Bence Jones-type side products.

In a specific embodiment said modification promoting the association of the first and the second sub-unit of the Fc domain is a so-called “knob-into-hole” modification, comprising a “knob” modification in one of the two sub-units of the Fc domain and a “hole” modification in the other one of the two sub-units of the Fc domain.

The knob-into-hole technology is described e.g. in U.S. Pat. Nos. 5,731,168; 7,695,936; Ridgway et al., Prot Eng 9, 617-621 (1996) and Carter, J Immunol Meth 248, 7-15 (2001). Generally, the method involves introducing a protuberance (“knob”) at the interface of a first polypeptide and a corresponding cavity (“hole”) in the interface of a second polypeptide, such that the protuberance can be positioned in the cavity so as to promote heterodimer formation and hinder homodimer formation. Protuberances are constructed by replacing small amino acid side chains from the interface of the first polypeptide with larger side chains (e.g. tyrosine or tryptophan). Compensatory cavities of identical or similar size to the protuberances are created in the interface of the second polypeptide by replacing large amino acid side chains with smaller ones (e.g. alanine or threonine).

Fc Domain Modifications Reducing Fc Receptor Binding and/or Effector Function

The Fc domain confers to the antibody or bispecific antibody favorable pharmacokinetic properties, including a long serum half-life which contributes to good accumulation in the target tissue and a favorable tissue-blood distribution ratio. At the same time it may, however, lead to undesirable targeting of the antibody or bispecific antibody to cells expressing Fc receptors rather than to the preferred antigen-bearing cells. Moreover, the co-activation of Fc receptor signaling pathways may lead to cytokine release which, in combination with the T cell activating properties (e.g. in embodiments of the bispecific antibody wherein the second antigen binding moiety binds to an activating T cell antigen) and the long half-life of the antibody or bispecific antibody, results in excessive activation of cytokine receptors and severe side effects upon systemic administration. Activation of (Fc receptor-bearing) immune cells other than T cells may even reduce efficacy of the bispecific antibody (particularly a bispecific antibody wherein the second antigen binding moiety binds to an activating T cell antigen) due to the potential destruction of T cells e.g. by NK cells.

Accordingly, in particular embodiments, the Fc domain of the antibody or bispecific antibody according to the invention may exhibit reduced binding affinity to an Fc receptor and/or reduced effector function, as compared to a native IgG₁ Fc domain. In one such embodiment the Fc domain (or the antibody or bispecific antibody comprising said Fc domain) exhibits less than 50%, preferably less than 20%, more preferably less than 10% and most preferably less than 5% of the binding affinity to an Fc receptor, as compared to a native IgG₁ Fc domain (or an antibody or bispecific antibody comprising a native IgG₁ Fc domain), and/or less than 50%, preferably less than 20%, more preferably less than 10% and most preferably less than 5% of the effector function, as compared to a native IgG₁ Fc domain domain (or an antibody or bispecific antibody comprising a native IgG₁ Fc domain). In one embodiment, the Fc domain domain (or the antibody or bispecific antibody comprising said Fc domain) does not substantially bind to an Fc receptor and/or induce effector function. In a particular embodiment the Fc receptor is an Fcγ receptor. In one embodiment the Fc receptor may be a human Fc receptor. In one embodiment the Fc receptor may be an activating Fc receptor. In a specific embodiment the Fc receptor may be an activating human Fcγ receptor, more specifically human FcγRIIIa, FcγRI or FcγRIIa, most specifically human FcγRIIIa. In one embodiment the effector function may be one or more selected from the group of CDC, ADCC, ADCP, and cytokine secretion. In a particular embodiment, the effector function may be ADCC. In one embodiment, the Fc domain domain may exhibit substantially similar binding affinity to neonatal Fc receptor (FcRn), as compared to a native IgG₁ Fc domain domain. Substantially similar binding to FcRn is achieved when the Fc domain (or the antibody or bispecific antibody comprising said Fc domain) exhibits greater than about 70%, particularly greater than about 80%, more particularly greater than about 90% of the binding affinity of a native IgG₁ Fc domain (or the antibody or bispecific antibody comprising a native IgG₁ Fc domain) to FcRn.

In certain embodiments the Fc domain may be engineered to have reduced binding affinity to an Fc receptor and/or reduced effector function, as compared to a non-engineered Fc domain. In particular embodiments, the Fc domain of the antibody or bispecific antibody may comprise one or more amino acid mutation that reduces the binding affinity of the Fc domain to an Fc receptor and/or effector function. Typically, the same one or more amino acid mutation is present in each of the two sub-units of the Fc domain. In one embodiment, the amino acid mutation may reduce the binding affinity of the Fc domain to an Fc receptor. In one embodiment, the amino acid mutation may reduce the binding affinity of the Fc domain to an Fc receptor by at least 2-fold, at least 5-fold, or at least 10-fold. In embodiments where there is more than one amino acid mutation that reduces the binding affinity of the Fc domain to the Fc receptor, the combination of these amino acid mutations may reduce the binding affinity of the Fc domain to an Fc receptor by at least 10-fold, at least 20-fold, or even at least 50-fold. In one embodiment the antibody or bispecific antibody comprising an engineered Fc domain exhibits less than 20%, particularly less than 10%, more particularly less than 5% of the binding affinity to an Fc receptor as compared to an antibody or bispecific antibody comprising a non-engineered Fc domain. In a particular embodiment, the Fc receptor may be an Fcγ receptor. In some embodiments, the Fc receptor may be a human Fc receptor.

In some embodiments, the Fc receptor may be an activating Fc receptor. In a specific embodiment, the Fc receptor may be an activating human Fcγ receptor, more specifically human FcγRIIIa, FcγRI or FcγRIIa, most specifically human FcγRIIIa. Preferably, binding to each of these receptors is reduced. In some embodiments, binding affinity to a complement component, specifically binding affinity to C1q, is also reduced. In one embodiment, binding affinity to neonatal Fc receptor (FcRn) is not reduced. Substantially similar binding to FcRn, i.e. preservation of the binding affinity of the Fc domain to said receptor, is achieved when the Fc domain (or the antibody or bispecific antibody comprising said Fc domain) exhibits greater than about 70% of the binding affinity of a non-engineered form of the Fc domain (or the antibody or bispecific antibody comprising said non-engineered form of the Fc domain) to FcRn. The Fc domain, or bispecific antibody of the invention comprising said Fc domain, may exhibit greater than about 80% and even greater than about 90% of such affinity. In certain embodiments, the Fc domain of the bispecific antibody may be engineered to have reduced effector function, as compared to a non-engineered Fc domain. The reduced effector function can include, but is not limited to, one or more of the following: reduced complement dependent cytotoxicity (CDC), reduced antibody-dependent cell-mediated cytotoxicity (ADCC), reduced antibody-dependent cellular phagocytosis (ADCP), reduced cytokine secretion, reduced immune complex-mediated antigen uptake by antigen-presenting cells, reduced binding to NK cells, reduced binding to macrophages, reduced binding to monocytes, reduced binding to polymorphonuclear cells, reduced direct signaling inducing apoptosis, reduced crosslinking of target-bound antibodies, reduced dendritic cell maturation, or reduced T cell priming. In one embodiment, the reduced effector function may be one or more selected from the group of reduced CDC, reduced ADCC, reduced ADCP, and reduced cytokine secretion. In a particular embodiment, the reduced effector function may be reduced ADCC. In one embodiment the reduced ADCC may be less than 20% of the ADCC induced by a non-engineered Fc domain (or a bispecific antibody comprising a non-engineered Fc domain).

In one embodiment, the amino acid mutation that reduces the binding affinity of the Fc domain to an Fc receptor and/or effector function may be an amino acid substitution. In one embodiment, the Fc domain may comprise an amino acid substitution at a position selected from the group of E233, L234, L235, N297, P331 and P329 (numberings according to Kabat EU index). In a more specific embodiment, the further amino acid substitution may be E233P, L234A, L235A, L235E, N297A, N297D or P331S, preferably L234A or/and L235A.

### Compositions, Formulations, and Routes of Administration

In a further aspect, the invention provides pharmaceutical compositions comprising any of the antibodies or bispecific antibody, e.g., for use in any of the below therapeutic methods. In one embodiment, a pharmaceutical composition comprises any of the antibodies or bispecific antibody provided herein and a pharmaceutically acceptable carrier. In another embodiment, a pharmaceutical composition comprises any of the antibodies or bispecific antibodies provided herein and at least one additional therapeutic agent, e.g., as described below.

Further provided is a method of producing an antibody or bispecific antibody of the invention in a form suitable for administration in vivo, the method comprising (a) obtaining an antibody or bispecific antibody according to the invention, and (b) formulating the antibody or bispecific antibody with at least one pharmaceutically acceptable carrier, whereby a preparation of antibody or bispecific antibody is formulated for administration in vivo.

Pharmaceutical compositions of the present invention comprise a therapeutically effective amount of antibody or bispecific antibody dissolved or dispersed in a pharmaceutically acceptable carrier. The phrases “pharmaceutical or pharmacologically acceptable” refers to molecular entities and compositions that are generally non-toxic to recipients at the dosages and concentrations employed, i.e. do not produce an adverse, allergic or other untoward reaction when administered to an animal, such as, for example, a human, as appropriate. The preparation of a pharmaceutical composition that contains an antibody or bispecific antibody and optionally an additional active ingredient will be known to those of skill in the art in light of the present disclosure, as exemplified by Remington's Pharmaceutical Sciences, 18th Ed. Mack Printing Company, 1990, incorporated herein by reference. Moreover, for animal (e.g., human) administration, it will be understood that preparations should meet sterility, pyrogenicity, general safety and purity standards as required by FDA Office of Biological Standards or corresponding authorities in other countries. Preferred compositions are lyophilized formulations or aqueous solutions. As used herein, “pharmaceutically acceptable carrier” includes any and all solvents, buffers, dispersion media, coatings, surfactants, antioxidants, preservatives (e.g. antibacterial agents, antifungal agents), isotonic agents, absorption delaying agents, salts, preservatives, antioxidants, proteins, drugs, drug stabilizers, polymers, gels, binders, excipients, disintegration agents, lubricants, sweetening agents, flavoring agents, dyes, such like materials and combinations thereof, as would be known to one of ordinary skill in the art (see, for example, Remington's Pharmaceutical Sciences, 18th Ed. Mack Printing Company, 1990, pp. 1289-1329, incorporated herein by reference). Except insofar as any conventional carrier is incompatible with the active ingredient, its use in the therapeutic or pharmaceutical compositions is contemplated.

An antibody or bispecific antibody of the invention (and any additional therapeutic agent) can be administered by any suitable means, including parenteral, intrapulmonary, and intranasal, and, if desired for local treatment, intralesional administration. Parenteral infusions include intramuscular, intravenous, intraarterial, intraperitoneal, or subcutaneous administration. Dosing can be by any suitable route, e.g. by injections, such as intravenous or subcutaneous injections, depending in part on whether the administration is brief or chronic.

Parenteral compositions include those designed for administration by injection, e.g. subcutaneous, intradermal, intralesional, intravenous, intraarterial intramuscular, intrathecal or intraperitoneal injection. For injection, the antibodies or bispecific antibodies of the invention may be formulated in aqueous solutions, preferably in physiologically compatible buffers such as Hanks' solution, Ringer's solution, or physiological saline buffer. The solution may contain formulatory agents such as suspending, stabilizing and/or dispersing agents. Alternatively, the antibodies or bispecific antibodies may be in powder form for constitution with a suitable vehicle, e.g., sterile pyrogen-free water, before use. Sterile injectable solutions are prepared by incorporating the antibodies or bispecific antibodies of the invention in the required amount in the appropriate solvent with various of the other ingredients enumerated below, as required. Sterility may be readily accomplished, e.g., by filtration through sterile filtration membranes.

Generally, dispersions are prepared by incorporating the various sterilized active ingredients into a sterile vehicle which contains the basic dispersion medium and/or the other ingredients. In the case of sterile powders for the preparation of sterile injectable solutions, suspensions or emulsion, the preferred methods of preparation are vacuum-drying or freeze-drying techniques which yield a powder of the active ingredient plus any additional desired ingredient from a previously sterile-filtered liquid medium thereof. The liquid medium should be suitably buffered if necessary and the liquid diluent first rendered isotonic prior to injection with sufficient saline or glucose. The composition must be stable under the conditions of manufacture and storage, and preserved against the contaminating action of microorganisms, such as bacteria and fungi. It will be appreciated that endotoxin contamination should be kept minimally at a safe level, for example, less that 0.5 ng/mg protein. Suitable pharmaceutically acceptable carriers include, but are not limited to: buffers such as phosphate, citrate, and other organic acids; antioxidants including ascorbic acid and methionine; preservatives (such as octadecyldimethylbenzyl ammonium chloride; hexamethonium chloride; benzalkonium chloride; benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as polyethylene glycol (PEG). Aqueous injection suspensions may contain compounds which increase the viscosity of the suspension, such as sodium carboxymethyl cellulose, sorbitol, dextran, or the like.

Optionally, the suspension may also contain suitable stabilizers or agents which increase the solubility of the compounds to allow for the preparation of highly concentrated solutions. Additionally, suspensions of the active compounds may be prepared as appropriate oily injection suspensions. Suitable lipophilic solvents or vehicles include fatty oils such as sesame oil, or synthetic fatty acid esters, such as ethyl cleats or triglycerides, or liposomes. Pharmaceutical compositions comprising the antibodies or bispecific antibodies of the invention may be manufactured by means of conventional mixing, dissolving, emulsifying, encapsulating, entrapping or lyophilizing processes. Pharmaceutical compositions may be formulated in conventional manner using one or more physiologically acceptable carriers, diluents, excipients or auxiliaries which facilitate processing of the proteins into preparations that can be used pharmaceutically. Proper formulation is dependent upon the route of administration chosen.

### Therapeutic Methods and Compositions

Any of the antibodies or bispecific antibodies provided herein may be used in therapeutic methods. Antibodies or bispecific antibodies of the invention may be used as immunotherapeutic agents, for example in the treatment of cancers.

For use in therapeutic methods, antibodies or bispecific antibodies of the invention would be formulated, dosed, and administered in a fashion consistent with good medical practice. Factors for consideration in this context include the particular disorder being treated, the particular mammal being treated, the clinical condition of the individual patient, the cause of the disorder, the site of delivery of the agent, the method of administration, the scheduling of administration, and other factors known to medical practitioners.

In one aspect, antibodies or bispecific antibodies of the invention for use as a medicament are provided. In further aspects, antibodies or bispecific antibodies of the invention for use in treating a disease are provided. In certain embodiments, antibodies or bispecific antibodies of the invention for use in a method of treatment are provided.

In one embodiment, the invention provides an antibody or bispecific antibodies as described herein for use in the treatment of a disease in an individual in need thereof. In certain embodiments, the invention provides an antibody or bispecific antibody for use in a method of treating an individual having a disease comprising administering to the individual a therapeutically effective amount of the antibody or bispecific antibody. In one embodiment the disease may be a cancer. In certain embodiments the method further comprises administering to the individual a therapeutically effective amount of at least one additional therapeutic agent, e.g., an anti-cancer agent if the disease to be treated is cancer. In further embodiments, the invention provides an antibody or bispecific antibody as described herein for use in inducing lysis of a target cell, particularly a tumor cell. In certain embodiments, the invention provides an antibody or bispecific antibody for use in a method of inducing lysis of a target cell, particularly a tumor cell, in an individual comprising administering to the individual an effective amount of the antibody or bispecific antibody to induce lysis of a target cell. An “individual” according to any of the above embodiments is a mammal, preferably a human.

In one embodiment, the disease to be treated may be a cancer.

In one embodiment, the cancer may be a solid cancer or a blood cancer.

In one embodiment, the cancer may be selected from the group consisting of leukemia, rectal cancer, endometrial cancer, nephroblastoma, basal cell carcinoma, nasopharyngeal cancer, bone tumor, esophageal cancer, lymphoma, Hodgkin's lymphoma, non-Hodgkin's lymphoma, follicular thyroid cancer, hepatocellular carcinoma, oral cancer, renal cell carcinoma, multiple myeloma, mesothelioma, osteosarcoma, myelodysplastic syndrome, mesenchymal tumor, soft tissue sarcoma, liposarcoma, gastrointestinal stromal tumor, malignant peripheral nerve sheath tumor (MPNST), ewing sarcoma, leiomyosarcoma, mesenchymal chondrosarcoma, lymphosarcoma, fibrosarcoma, rhabdomyosarcoma, teratoma, neuroblastoma, medulloblastoma, glioma, benign skin tumor, Burkitt's lymphoma, mantle cell lymphoma, diffuse large B cell lymphoma (DLBCL), follicular lymphoma, marginal zone lymphoma, neuroectodermal tumor, epithelial tumor, cutaneous T-cell lymphoma (CTCL), peripheral T cell lymphoma (PTCL), pancreas cancer, haematological malignancies, kidney cancer, tumor vasculature, breast cancer, renal cancer, ovarian cancer, epithelial ovarian cancer, gastric cancer, liver cancer, lung cancer, colorectal cancer, pancreatic cancer, skin cancer, bladder cancer, testicular cancer, uterine cancer, prostate cancer, small cell lung cancer (SCLC), non-small cell lung cancer (NSCLC), neuroblastoma, brain cancer, colon cancer, squamous cell carcinoma, melanoma, myeloma, cervical cancer, thyroid cancer, head and neck cancer and adrenal cancer.

In one embodiment, the leukemia may be selected from the group consisting of acute lymphoblastic leukemia (ALL), chronic lymphocytic leukemia (CLL), hairy-cell leukemia, myelodysplastic syndrome (MDS), chronic myelogenous leukemia (CML) and acute myeloid leukemia (AML), preferably AML.

In one embodiment, the cancer may be a cancer expressing CLL-1.

A skilled artisan readily recognizes that in many cases the antibody or bispecific antibody may not provide a cure but may only provide partial benefit. In some embodiments, a physiological change having some benefit is also considered therapeutically beneficial. Thus, in some embodiments, an amount of antibody or bispecific antibody that provides a physiological change is considered an “effective amount” or a “therapeutically effective amount”. The subject, patient, or individual in need of treatment is typically a mammal, more specifically a human.

In some embodiments, an effective amount of an antibody or bispecific antibody of the invention is administered to a cell. In other embodiments, a therapeutically effective amount of an antibody or bispecific antibody of the invention is administered to an individual for the treatment of disease.

For the prevention or treatment of disease, the appropriate dosage of an antibody or bispecific antibody of the invention (when used alone or in combination with one or more other additional therapeutic agents) will depend on the type of disease to be treated, the route of administration, the body weight of the patient, the type of antibody or bispecific antibody, the severity and course of the disease, whether the antibody or bispecific antibody is administered for preventive or therapeutic purposes, previous or concurrent therapeutic interventions, the patient's clinical history and response to the antibody or bispecific antibody, and the discretion of the attending physician. The practitioner responsible for administration will, in any event, determine the concentration of active ingredient(s) in a composition and appropriate dose(s) for the individual subject. Various dosing schedules including but not limited to single or multiple administrations over various time-points, bolus administration, and pulse infusion are contemplated herein.

The antibody or bispecific antibody is suitably administered to the patient at one time or over a series of treatments. Depending on the type and severity of the disease, about 1 ng/kg to 100 mg/kg of antibody or bispecific antibody can be administered to the patient.

Hereafter, the present disclosure will be described in detail with examples.

The following examples are intended merely to illustrate the disclosure and are not to be construed to restrict the disclosure.

### Example 1: Preparation of Anti-CLL-1 Antibodies

### 1.1. Monoclonal Antibody Screening by Mouse Immunization

To prepare anti-CLL-1 antibodies, mouse immunization was performed to screen monoclonal antibodies.

Briefly, two groups of mice (SJL and Balb/c mice from Charles River Laboratories, Hollister, CA) were immunized by being mixed with human and cynomolgus CLL1 with N-terminus human IgG1 Fc fusion proteins (the mice were produced in-house, and the amino acid sequences of the antigen are shown in Table 4).

Through ELISA (enzyme-linked immunosorbent assay) screening and FACS (fluorescence-activated cell sorting) analysis, three hybridoma clones were screened as human and cynomolgus CLL1 cross-reactive clones. The three clones were 16C6, 33C2 and 84A2 from immunized Balb/c mice group. Also, all clones positively bound to CLL1 expressing cell lines (U937, HL60) and a cell line overexpressing CLL-1 (cynomolgus CLL1 and human CLL1 are overexpressed in HEK293E cells).

TABLE 4

| | | SEQ |
|---|---|---|
| Name | Amino acid sequence of antigens for mouse immunization | ID NO: |
| hFc- | MYRMQLLSCIALSLALVTNSAPLEPKSSDKTHTCPPCPAPELLGGPSVFLFPPKPKD | 72 |
| Human | TLMISRTPEVTCVVVDVSHEDPEVKFNWYVDGVEVHNAKTKPREEQYNSTYRVVSVL | |
| CLL1 | TVLHQDWLNGKEYKCKVSNKALPAPIEKTISKAKGQPREPQVYTLPPSREEMTKNQV | |
| | SLTCLVKGFYPSDIAVEWESNGQPENNYKTTPPVLDSDGSFFLYSKLTVDKSRWQQG | |
| | NVFSCSVMHEALHNHYTQKSLSLSPGARTGGGGSGGGGSHVTLKIEMKKMNKLQNIS | |
| | EELQRNISLQLMSNMNISNKIRNLSTTLQTIATKLCRELYSKEQEHKCKPCPRRWIW | |
| | HKDSCYFLSDDVQTWQESKMACAAQNASLLKINNKNALEFIKSQSRSYDYWLGLSPE | |
| | EDSTRGMRVDNIINSSAWVIRNAPDLNNMYCGYINRLYVQYYHCTYKKRMICEKMAN | |
| | PVQLGSTYFREA | |
| hFc- | MYRMQLLSCIALSLALVTNSAPLEPKSSDKTHTCPPCPAPELLGGPSVFLFPPKPKD | 73 |
| Cyno- | TLMISRTPEVTCVVVDVSHEDPEVKFNWYVDGVEVHNAKTKPREEQYNSTYRVVSVL | |
| molgus | TVLHQDWLNGKEYKCKVSNKALPAPIEKTISKAKGQPREPQVYTLPPSREEMTKNQV | |
| CLL1 | SLTCLVKGFYPSDIAVEWESNGQPENNYKTTPPVLDSDGSFFLYSKLTVDKSRWQQG | |
| | NVFSCSVMHEALHNHYTQKSLSLSPGARTGGGGSGGGGSHITLKTAMKKMNKLQNIN | |
| | EELQRNVSLQLMSNMNSSNKIRNLSTTLQTIATRLCRELYSKEQEHKCKPCPRRWIW | |
| | HKDSCYFLSDDVRTWQESRMACAAQNASLLKINNKNALEFIKSQSTSYPYWLGLSPE | |
| | KDYSYGTSVDDIINSSAWVTRNASDLNNMFCGYINRIYVHYDYCIYRKKMICEKMAN | |
| | PVQLGFIHFREA | |

1.2. Monoclonal Antibody Sequencing from Hybridoma Cells and Antibody Chimerization

To sequence 16C6, 33C2 and 84A2 from immunized Balb/c mice group in Example 1.1., monoclonal antibodies from hybridoma cells were sequenced.

Briefly, total RNA of murine 16C6, 33C2 and 84A2 was isolated from the hybridoma cells following the technical manual of Trizol reagent (Ambion, Cat. No.: 15596-026). Total RNA was then reverse-transcribed into cDNA through RT-PCR using either isotype-specific anti-sense primers or universal primers. Antibody fragments of VH (variable heavy chain) and VL (variable light chain) were amplified by using the rapid amplification of cDNA ends (RACE) technique. Amplified antibody fragments were cloned into cloning vectors and then were subjected to sequencing. Variable regions and CDRs of the three CLL1 positive clones are shown in Table 5, Table 6 and Table 7. In the tables, HCDR is CDR in heavy chain, LCDR is CDR in light chain.

TABLE 5

| | | SEQ |
|---|---|---|
| 16C6 | Amino acid sequence | ID NO: |
| Variable | EVQLQQSGPELVKPGASVKISCKASGYSFTGYYMHWVKQSHVKSLEWIGHVNPY | 25 |
| Heavy | NGATSFNRNFKDKASLTVDKSSSTAYMELHSLTSEDSAVYYCGRSADGYYERHF | |
| Chain | DYWGQGTTLTVSS | |
| HCDR1 | GYYMH | 1 |
| HCDR2 | HVNPYNGATSFNRNFKD | 4 |
| HCDR3 | SADGYYERHFDY | 7 |
| Variable | DIVLTQSPASLAVSLGQRATISCKASQSVDYDGDSYMNWYQQKPGQPPKLLIYA | 36 |
| Light | ASNLQSGIPARFSGSGSGTDFTLNIHPVEEEDAATYYCQQSDKDPLTFGAGTKL | |
| Chain | ELK | |
| LCDR1 | KASQSVDYDGDSYMN | 12 |
| LCDR2 | AASNLQS | 16 |
| LCDR3 | QQSDKDPLT | 19 |

TABLE 6

| | | SEQ |
|---|---|---|
| 33C2 | Amino acid sequence | ID NO: |
| Variable | EVQLQQSGPELVKPGASMKISCKASGYSFTGYAMNWVKQSHGKNLEWIGLINPY | 26 |
| Heavy | NGGAMYNQKFKGKATLTVDKSTSTAYMELLSLTSEDSAVYYCARDYRYDGHLDY | |
| Chain | WGQGTTLTVSS | |
| HCDR1 | GYAMN | 2 |
| HCDR2 | LINPYNGGAMYNQKFKG | 5 |
| HCDR3 | DYRYDGHLDY | 8 |
| Variable | QIVLTQSPVIMSASPGEKVTMTCSATSSVSYMHWYQQKSGTSPKRWIYDTSKLA | 37 |
| Light | SGVPVRFSGSGSGTSYSLTISRMEAEDAATYYCQQWSSDSPTFGVGTKLELK | |
| Chain | | |
| LCDR1 | SATSSVSYMH | 13 |
| LCDR2 | DTSKLAS | 17 |
| LCDR3 | QQWSSDSPT | 20 |

TABLE 7

| | | SEQ |
|---|---|---|
| 84A2 | Amino acid sequence | ID NO: |
| Variable | EVQLQQSGPELVKSGASVKMSCKASGYTFTSYVIHWVKQMPGQGLEWIGLFNPY | 27 |
| Heavy | NDDVNYNEKFKGKATLTSDKYSSTAYLDLSSLTSEDSAVYYCAREGVHYGRPWF | |
| Chain | GYWGQGTLVTVSA | |
| HCDR1 | SYVIH | 3 |
| HCDR2 | LFNPYNDDVNYNEKFKG | 6 |
| HCDR3 | EGVHYGRPWFGY | 9 |
| Variable | DIKMTQSPSSMNASLGERVTITCKASQDIKSYLNWFQQKPGKSPKTLIYRASRL | 38 |
| Light | VDGVPARFSGSESGQDYSLTISSLEFEDMGIYYCLQYDEFPLTFGAGTKLELK | |
| Chain | | |
| LCDR1 | KASQDIKSYLN | 14 |
| LCDR2 | RASRLVD | 18 |
| LCDR3 | LQYDEFPLT | 21 |

To generate a chimeric antibody, the VH and VL of murine antibody were combined with a human IgG1 heavy chain constant region and a human kappa light chain constant region, respectively and cloned into an expression vector. The constant region of the chimeric antibody was modified by introducing more than one mutation or change (e.g., N297A (also referred to as ‘NA’) to null ADCC activity) into the human IgG1. Then, three modified chimeric antibodies of three clones were additionally modified to remove the post translational modification (PTM) or the N-glycosylation residues by using a site-directed mutagenesis. The modified sequences of 16C6, 33C2 and 84A2 as described above are shown in Table 8, Table 9, and Table 10. Hereinafter, ch16C6 refers to a chimeric antibody of 16C6 or 16C6(NA), which comprises a heavy chain having N297A mutant, ch33C2 refers to a chimeric antibody of 33C2 or 33C2(NA), which comprises a heavy chain having N297A mutant, and ch84C2 refers to a chimeric antibody of 84C2 or ch84A2(NA/N12S), which comprises a heavy chain having N297A mutant and a light chain having N12S mutant.

TABLE 8

| | | SEQ |
|---|---|---|
| ch16C6 | Amino acid sequence | ID NO: |
| Variable | EVQLQQSGPELVKPGASVKISCKASGYSFTGYYMHWVKQSHVKSLEWIGHVNPY | 28 |
| Heavy | NGATSFNRNFKDKASLTVDKSSSTAYMELHSLTSEDSAVYYCGRSAYGYYERHF | |
| Chain | DYWGQGTTLTVSS | |
| Constant | ASTKGPSVFPLAPSSKSTSGGTAALGCLVKDYFPEPVTVSWNSGALTSGVHTFP | 23 |
| region | AVLQSSGLYSLSSVVTVPSSSLGTQTYICNVNHKPSNTKVDKKVEPKSCDKTHT | |
| of Heavy | CPPCPAPELLGGPSVFLFPPKPKDTLMISRTPEVTCVVVDVSHEDPEVKFNWYV | |
| chain | DGVEVHNAKTKPREEQYNSTYRVVSVLTVLHQDWLNGKEYKCKVSNKALPAPIE | |
| | KTISKAKGQPREPQVYTLPPSREEMTKNQVSLTCLVKGFYPSDIAVEWESNGQP | |
| | ENNYKTTPPVLDSDGSFFLYSKLTVDKSRWQQGNVFSCSVMHEALHNHYTQKSL | |
| | SLSPGK | |
| Constant | ASTKGPSVFPLAPSSKSTSGGTAALGCLVKDYFPEPVTVSWNSGALTSGVHTFP | 24 |
| region | AVLQSSGLYSLSSVVTVPSSSLGTQTYICNVNHKPSNTKVDKKVEPKSCDKTHT | |
| of Heavy | CPPCPAPELLGGPSVFLFPPKPKDTLMISRTPEVTCVVVDVSHEDPEVKFNWYV | |
| chain | DGVEVHNAKTKPREEQYASTYRVVSVLTVLHQDWLNGKEYKCKVSNKALPAPIE | |
| N297A | KTISKAKGQPREPQVYTLPPSREEMTKNQVSLTCLVKGFYPSDIAVEWESNGQP | |
| mutant | ENNYKTTPPVLDSDGSFFLYSKLTVDKSRWQQGNVFSCSVMHEALHNHYTQKSL | |
| (NA) | SLSPGK | |
| HCDR1 | GYYMH | 1 |
| HCDR2 | HVNPYNGATSFNRNFKD | 4 |
| HCDR3 | SAYGYYERHFDY | 10 |
| Variable | DIVLTQSPASLAVSLGQRATISCKASQSVDYDADSYMNWYQQKPGQPPKLLIYA | 39 |
| Light | ASNLQSGIPARFSGSGSGTDFTLNIHPVEEEDAATYYCQQSDRDPLTFGAGTKL | |
| Chain | ELK | |
| Constant | RTVAAPSVFIFPPSDEQLKSGTASVVCLLNNFYPREAKVQWKVDNALQSGNSQE | 55 |
| region | SVTEQDSKDSTYSLSSTLTLSKADYEKHKVYACEVTHQGLSSPVTKSFNRGEC | |
| of Light | | |
| chain | | |
| LCDR1 | KASQSVDYDADSYMN | 15 |
| LCDR2 | AASNLQS | 16 |
| LCDR3 | QQSDRDPLT | 22 |

TABLE 9

| | | SEQ |
|---|---|---|
| ch33C2 | Amino acid sequence | ID NO: |
| Variable | EVQLQQSGPELVKPGASMKISCKASGYSFTGYAMNWVKQSHGKNLEWIGLINPY | 29 |
| Heavy | NGGAMYNQKFKGKATLTVDKSTSTAYMELLSLTSEDSAVYYCARDYRYEGHLDY | |
| Chain | WGQGTTLTVSS | |
| Constant | ASTKGPSVFPLAPSSKSTSGGTAALGCLVKDYFPEPVTVSWNSGALTSGVHTFP | 23 |
| region | AVLQSSGLYSLSSVVTVPSSSLGTQTYICNVNHKPSNTKVDKKVEPKSCDKTHT | |
| of Heavy | CPPCPAPELLGGPSVFLFPPKPKDTLMISRTPEVTCVVVDVSHEDPEVKFNWYV | |
| chain | DGVEVHNAKTKPREEQYNSTYRVVSVLTVLHQDWLNGKEYKCKVSNKALPAPIE | |
| | KTISKAKGQPREPQVYTLPPSREEMTKNQVSLTCLVKGFYPSDIAVEWESNGQP | |
| | ENNYKTTPPVLDSDGSFFLYSKLTVDKSRWQQGNVFSCSVMHEALHNHYTQKSL | |
| | SLSPGK | |
| Constant | ASTKGPSVFPLAPSSKSTSGGTAALGCLVKDYFPEPVTVSWNSGALTSGVHTFP | 24 |
| region | AVLQSSGLYSLSSVVTVPSSSLGTQTYICNVNHKPSNTKVDKKVEPKSCDKTHT | |
| of Heavy | CPPCPAPELLGGPSVFLFPPKPKDTLMISRTPEVTCVVVDVSHEDPEVKFNWYV | |
| chain | DGVEVHNAKTKPREEQYASTYRVVSVLTVLHQDWLNGKEYKCKVSNKALPAPIE | |
| N297A | KTISKAKGQPREPQVYTLPPSREEMTKNQVSLTCLVKGFYPSDIAVEWESNGQP | |
| mutant | ENNYKTTPPVLDSDGSFFLYSKLTVDKSRWQQGNVFSCSVMHEALHNHYTQKSL | |
| (NA) | SLSPGK | |
| HCDR1 | GYAMN | 2 |
| HCDR2 | LINPYNGGAMYNQKFKG | 5 |
| HCDR3 | DYRYEGHLDY | 11 |
| Variable | QIVLTQSPVIMSASPGEKVTMTCSATSSVSYMHWYQQKSGTSPKRWIYDTSKLA | 37 |
| Light | SGVPVRFSGSGSGTSYSLTISRMEAEDAATYYCQQWSSDSPTFGVGTKLELK | |
| Chain | | |
| Constant | RTVAAPSVFIFPPSDEQLKSGTASVVCLLNNFYPREAKVQWKVDNALQSGNSQE | 55 |
| region | SVTEQDSKDSTYSLSSTLTLSKADYEKHKVYACEVTHQGLSSPVTKSFNRGEC | |
| of Light | | |
| chain | | |
| LCDR1 | SATSSVSYMH | 13 |
| LCDR2 | DTSKLAS | 17 |
| LCDR3 | QQWSSDSPT | 20 |

TABLE 10

| | | SEQ |
|---|---|---|
| ch84C2 | Amino acid sequence | ID NO: |
| Variable | EVQLQQSGPELVKSGASVKMSCKASGYTFTSYVIHWVKQMPGQGLEWIGLFNPY | 27 |
| Heavy | NDDVNYNEKFKGKATLTSDKYSSTAYLDLSSLTSEDSAVYYCAREGVHYGRPWF | |
| Chain | GYWGQGTLVTVSA | |
| Constant | ASTKGPSVFPLAPSSKSTSGGTAALGCLVKDYFPEPVTVSWNSGALTSGVHTFP | 23 |
| region | AVLQSSGLYSLSSVVTVPSSSLGTQTYICNVNHKPSNTKVDKKVEPKSCDKTHT | |
| of Heavy | CPPCPAPELLGGPSVFLFPPKPKDTLMISRTPEVTCVVVDVSHEDPEVKFNWYV | |
| chain | DGVEVHNAKTKPREEQYNSTYRVVSVLTVLHQDWLNGKEYKCKVSNKALPAPIE | |
| | KTISKAKGQPREPQVYTLPPSREEMTKNQVSLTCLVKGFYPSDIAVEWESNGQP | |
| | ENNYKTTPPVLDSDGSFFLYSKLTVDKSRWQQGNVFSCSVMHEALHNHYTQKSL | |
| | SLSPGK | |
| Constant | ASTKGPSVFPLAPSSKSTSGGTAALGCLVKDYFPEPVTVSWNSGALTSGVHTFP | 24 |
| region | AVLQSSGLYSLSSVVTVPSSSLGTQTYICNVNHKPSNTKVDKKVEPKSCDKTHT | |
| of Heavy | CPPCPAPELLGGPSVFLFPPKPKDTLMISRTPEVTCVVVDVSHEDPEVKFNWYV | |
| chain | DGVEVHNAKTKPREEQYASTYRVVSVLTVLHQDWLNGKEYKCKVSNKALPAPIE | |
| N297A | KTISKAKGQPREPQVYTLPPSREEMTKNQVSLTCLVKGFYPSDIAVEWESNGQP | |
| mutant | ENNYKTTPPVLDSDGSFFLYSKLTVDKSRWQQGNVFSCSVMHEALHNHYTQKSL | |
| (NA) | SLSPGK | |
| HCDR1 | SYVIH | 3 |
| HCDR2 | LFNPYNDDVNYNEKFKG | 6 |
| HCDR3 | EGVHYGRPWFGY | 9 |
| Variable | DIKMTQSPSSMsASLGERVTITCKASQDIKSYLNWFQQKPGKSPKTLIYRASRL | 40 |
| Light | VDGVPARFSGSESGQDYSLTISSLEFEDMGIYYCLQYDEFPLTFGAGTKLELK | |
| Chain(N12S) | | |
| Constant | RTVAAPSVFIFPPSDEQLKSGTASVVCLLNNFYPREAKVQWKVDNALQSGNSQE | 55 |
| region of | SVTEQDSKDSTYSLSSTLTLSKADYEKHKVYACEVTHQGLSSPVTKSFNRGEC | |
| Light | | |
| chain | | |
| LCDR1 | KASQDIKSYLN | 14 |
| LCDR2 | RASRLVD | 18 |
| LCDR3 | LQYDEFPLT | 21 |

### 1.3. Antibody Humanization

To humanize the chimeric antibodies of Example 1.2., homology modeling was performed.

Briefly, through homology modeling, the modeled structure of the murine antibody was obtained, and the residues needed for a back mutation were analyzed. A human acceptor framework for VH and VL that has the highest sequence identity with the mouse counterpart was selected. Then, a complementarity-determining region (CDR) of a murine antibody was grafted into the human acceptor framework. Next, a rational back mutation design of the grafted antibody was performed. The designed humanized antibodies were produced and their binding property was evaluated. Among the mouse and chimerized clones, 16C6 and 33C2 were chosen as lead candidate antibodies. Especially, the humanized antibodies having CDRs of chimeric antibodies (i.e., PTM or N-glycosylation residue have been removed by additional modification) are named as 16C6(M14) and 33C2(M12). The sequences of humanized heavy and light chains of 16C6, 16C6(M14), 33C2 and 33C2(M12) are shown in Tables 11 to 14. Hereinafter, hu16C6 and hu16C6(M14) refers to a humanized antibodies of 16C6 and 16C6(M14), respectively, and hu33C2 and 33C2(M12) refers to a humanized antibodies of 33C2 and 33C2(M12), respectively.

TABLE 11

| | | | SEQ |
|---|---|---|---|
| | | | ID |
| hu16C6 | | Amino acid sequence | NO: |
| Variable | VH1 | QVQLVQSGAEVKKPGASVKVSCKASGYSFTGYYMHWVRQAPGQGLEWM | 30 |
| Heavy | | GHVNPYNGATSFNRNFKDRVTMTRDTSTSTVYMELSSLRSEDTAVYYC | |
| Chain | | ARSADGYYERHFDYWGQGTLVTVSS | |
| | VH2 | QVQLVQSGAEVKKPGASVKVSCKASGYSFTGYYMHWVRQAPGQGLEWI | 31 |
| | | GHVNPYNGATSFNRNFKDRVTMTVDTSTSTVYMELSSLRSEDTAVYYC | |
| | | GRSADGYYERHFDYWGQGTLVTVSS | |
| | VH6 | QVQLVQSGAEVKKPGASVKISCKASGYSFTGYYMHWVKQSHGQGLEWI | 32 |
| | | GHVNPYNGATSFNRNFKDRASLTVDTSTSTVYMELSSLRSEDSAVYYC | |
| | | GRSADGYYERHFDYWGQGTLVTVSS | |

| HCDR1 | GYYMH | | 1 |
|---|---|---|---|
| HCDR2 | HVNPYNGATSFNRNFKD | | 4 |
| HCDR3 | SADGYYERHFDY | | 7 |
| Variable | VL1 | DIVMTQSPDSLAVSLGERATINCKASQSVDYDGDSYMNWYQQKPGQPP | 41 |
| Light | | KLLIYAASNLQSGVPDRFSGSGSGTDFTLTISSLQAEDVAVYYCQQSD | |
| Chain | | KDPLTFGGGTKLEIK | |

| | | |
|---|---|---|
| LCDR1 | KASQSVDYDGDSYMN | 12 |
| LCDR2 | AASNLQS | 16 |
| LCDR3 | QQSDKDPLT | 19 |

TABLE 12

| | | | SEQ |
|---|---|---|---|
| | | | ID |
| hu16C6(M14) | | Amino acid sequence | NO: |
| Variable | VH1 | QVQLVQSGAEVKKPGASVKISCKASGYSFTGYYMHWVKQSHGQGLEWI | 74 |
| Heavy | | GHVNPYNGATSFNRNFKDRASLTVDTSTSTVYMELSSLRSEDSAVYYC | |
| Chain | | GRSAYGYYERHFDYWGQGTLVTVSS | |
| HCDR1 | GYYMH | | 1 |
| HCDR2 | HVNPYNGATSFNRNFKD | | 4 |
| HCDR3 | SAYGYYERHFDY | | 10 |
| Variable | VL1 | DIVMTQSPDSLAVSLGERATINCKASQSVDYDADSYMNWYQQKPGQPP | 75 |
| Light | | KLLIYAASNLQSGVPDRFSGSGSGTDFTLTISSLQAEDVAVYYCQQSD | |
| Chain | | RDPLTFGGGTKLEIK | |

| | | |
|---|---|---|
| LCDR1 | KASQSVDYDADSYMN | 15 |
| LCDR2 | AASNLQS | 16 |
| LCDR3 | QQSDRDPLT | 22 |

TABLE 13

| | | | SEQ |
|---|---|---|---|
| | | | ID |
| hu33C2 | | Amino acid sequence | NO: |
| Variable | VH3 | QVQLVQSGAEVKKPGASVKVSCKASGYSFTGYAMNWVRQAPGQRLEW | 33 |
| Heavy | | IGLINPYNGGAMYNQKFKGRATLTVDTSASTAYMELSSLRSEDTAVY | |
| Chain | | YCARDYRYDGHLDYWGQGTLVTVSS | |
| | VH4 | QVQLVQSGAEVKKPGASVKISCKASGYSFTGYAMNWVKQAPGQRLEW | 35 |
| | | IGLINPYNGGAMYNQKFKGRATLTVDTSASTAYMELSSLRSEDTAVY | |
| | | YCARDYRYDGHLDYWGQGTLVTVSS | |
| HCDR1 | GYAMN | | 2 |
| HCDR2 | LINPYNGGAMYNQKFKG | | 5 |
| HCDR3 | DYRYDGHLDY | | 8 |
| Variable | VL6 | EIVLTQSPATLSLSPGERATLSCSATSSVSYMHWYQQKPGQAPRRWI | 42 |
| Light | | YDTSKLASGIPARFSGSGSGTSYTLTISSLEPEDFAVYYCQQWSSDS | |
| Chain | | PTFGQGTKLEIK | |
| | VL7 | EIVLTQSPATLSLSPGERATMSCSATSSVSYMHWYQQKPGTAPRRWI | 43 |
| | | YDTSKLASGIPARFSGSGSGTDYTLTISSLEPEDFAVYYCQQWSSDS | |
| | | PTFGQGTKLEIK | |
| | VL8 | EIVLTQSPATLSLSPGERATLSCSATSSVSYMHWYQQKSGTAPRRWI | 44 |
| | | YDTSKLASGIPARFSGSGSGTDYTLTISSLEPEDAAVYYCQQWSSDS | |
| | | PTFGQGTKLEIK | |

| | | |
|---|---|---|
| LCDR1 | SATSSVSYMH | 13 |
| LCDR2 | DTSKLAS | 17 |
| LCDR3 | QQWSSDSPT | 20 |

TABLE 14

| | | | SEQ |
|---|---|---|---|
| | | | ID |
| hu33C2(M12) | | Amino acid sequence | NO: |
| Variable | VH3 | QVQLVQSGAEVKKPGASVKVSCKASGYSFTGYAMNWVRQAPGQRLEW | 34 |
| Heavy | | IGLINPYNGGAMYNQKFKGRATLTVDTSASTAYMELSSLRSEDTAVY | |
| Chain | | YCARDYRYEGHLDYWGQGTLVTVSS | |
| HCDR1 | GYAMN | | 2 |
| HCDR2 | LINPYNGGAMYNQKFKG | | 5 |
| HCDR3 | DYRYEGHLDY | | 11 |
| Variable | VL6 | EIVLTQSPATLSLSPGERATLSCSATSSVSYMHWYQQKPGQAPRRWI | 42 |
| Light | | YDTSKLASGIPARFSGSGSGTSYTLTISSLEPEDFAVYYCQQWSSDS | |
| Chain | | PTFGQGTKLEIK | |

| | | |
|---|---|---|
| LCDR1 | SATSSVSYMH | 13 |
| LCDR2 | DTSKLAS | 17 |
| LCDR3 | QQWSSDSPT | 20 |

### Example 2. Evaluation of Activity of Anti-CLL-1 Monospecific Antibodies

### 2.1. Ligand Binding Activity Test (ELISA)

Antibody candidates of Example 1.2. were analyzed to compare the ligand binding activity by ELISA, using huCLL1-His and hFc-huCLL1 as ligands.

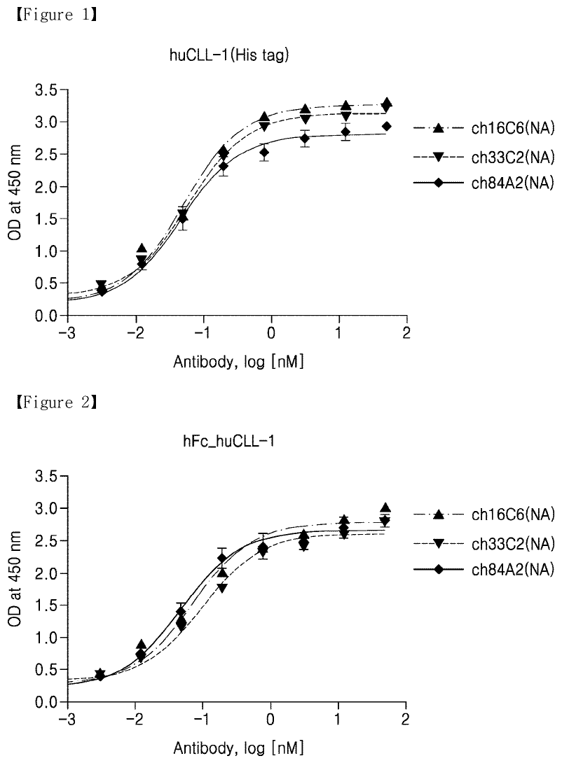

Briefly, the antigen (ligand) was coated overnight at 4VC. The antigen coated plate was blocked with 1% BSA in PBS at 4VC for 2 hr and incubated with antibodies at 4° C. for 2 hrs. To find out the EC₅₀ (nM) of each antibody, 4-fold serial dilutions were prepared from an antibody solution of a concentration of 50 nM to 0.2 pM and diluted antibodies were treated to each well, and the plate was washed with 1×PBST. Then Goat anti-Human IgG F(ab′)2 cross-adsorbed secondary antibody conjugated with HRP (horseradish peroxidase) (Thermo, 31414) was added to each well and incubated at 4° C. for 1 hr. After wash, a TMB (3,3′,5,5′-tetramethylbenzidine) substrate (Sigma, T0440) was added to each well and the OD at 450 nm was measured by an ELISA plate reader. The results are shown in FIGS. 1 and 2.

FIG. 1 is a graph showing ligand binding activity of three chimeric antibodies according to an embodiment using huCLL1-His as a ligand.

FIG. 2 is a graph showing ligand binding activity of three chimeric antibodies according to an embodiment using hFc-huCLL1 as a ligand.

As shown in FIGS. 1 and 2, it was confirmed that all three chimeric antibodies according to an embodiment bound strongly to both human CLL1-His and hFc-human CLL1 antigen.

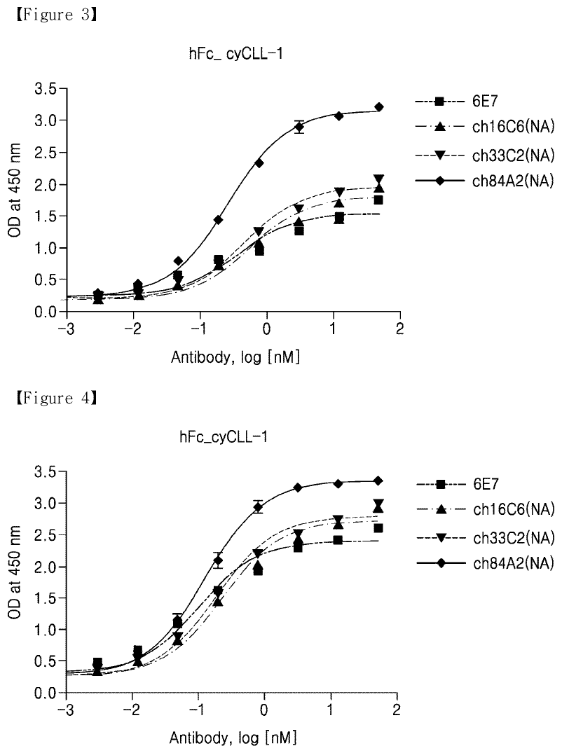

Continuously, antibody candidates of Example 1.2. were analyzed to compare the ligand binding activities by ELISA, using 50 ng/well and 100 ng/well of hFc-Cynomolgus CLL1 as a ligandin the same manner as described above. Genentech's anti-CLL-1 antibody 6E7 was used as a reference. The results are shown in FIGS. 3 and 4.

FIG. 3 is a graph showing ligand binding activity of three chimeric antibodies according to an embodiment using 50 ng/well of hFc-Cynomolgus CLL1 as a ligand.

FIG. 4 is a graph showing ligand binding activity of three chimeric antibodies according to an embodiment using 100 ng/well of hFc-Cynomolgus CLL1 as a ligand.

As shown in FIGS. 3 and 4, it was confirmed that all three chimeric antibodies according to an embodiment bound strongly to the hFc-cynomolgus CLL1 regardless of the antigen concentration. Also, it was confirmed that all three chimeric antibodies according to an embodiment has a stronger ligand binding activity than that of Genentech's antibody, 6E7.

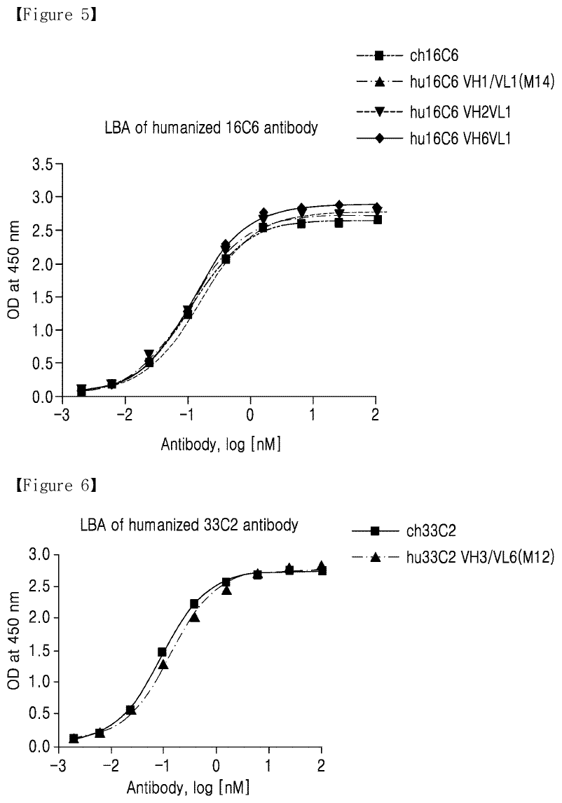

The ligand binding activities of the chimeric antibody of Example 1.2. and the humanized antibody of Example 1.3. were compared in the same manner as described above. The results are shown in FIGS. 5, 6 and 7.

FIG. 5 is a graph showing ligand binding activity of a chimeric antibody and humanized antibodies hu16C6 according to an embodiment.

FIG. 6 is a graph showing ligand binding activity of a chimeric antibody and humanized antibodies hu33C2 according to an embodiment.

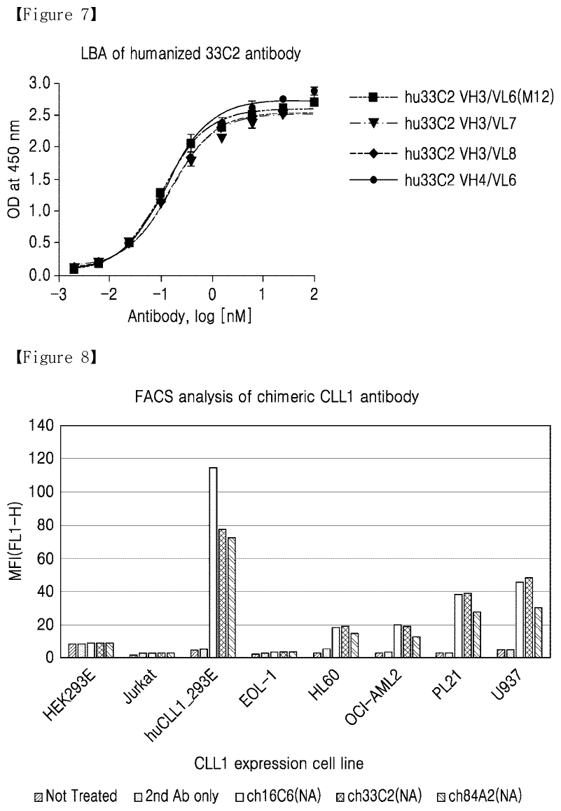

FIG. 7 is a graph showing ligand binding activity of various humanized antibodies hu33C2 according to an embodiment.

As shown in FIGS. 5 to 7, the humanized antibody according to an embodiment exhibit binding affinity equivalent to that of the chimeric antibody according to an embodiment. Since humanization of murine or chimeric antibodies usually results in reduced binding affinities or the binding affinity is even lost, these results demonstrate that the humanized antibodies according to an embodiment are advantageous.

### 2.2. Cell Binding Activity Test (FACS)

To evaluate cell binding property, the chimeric antibody candidates of Example 1.2. were analyzed in CLL1 negative cells (HEK293E and Jurkat) and various CLL1 expressing cancer cells by using FACS.

Briefly, cells were incubated with antibodies at 4° C. for 1 hr. Firstly, the cells were washed with an assay buffer (1% BSA in PBS). Then, an anti-human IgG Fc conjugated with FITC (Fluorescein isothiocyanate) (Sigma, F9512) was added to each well and incubated at 4° C. for 1 hr. After wash, the MFI (Median Fluorescence Intensity) of FITC was measured by BD FACS Cal ibur (BD Biosciences).

The results are shown in Table 15 and FIG. 8.

TABLE 15

| | | | | | | OCI- | | |
|---|---|---|---|---|---|---|---|---|
| MFI(FL1-H) | HEK293E | Jurkat | huCLL1_293E | EOL-1 | HL60 | AML2 | PL21 | U937 |
| Not Treated | 8.18 | 1.44 | 4.59 | 2.29 | 2.85 | 2.67 | 2.52 | 4.66 |
| 2nd Ab only | 8.51 | 2.7 | 5.05 | 2.53 | 5.09 | 3.28 | 2.82 | 4.77 |
| ch16C6(NA) | 8.82 | 2.8 | 115 | 3.73 | 18.5 | 19.5 | 38.6 | 45.8 |
| ch33C2(NA) | 8.81 | 2.75 | 77.7 | 3.6 | 19.1 | 19.3 | 38.8 | 48.1 |
| ch84A2(NA) | 8.76 | 2.69 | 72.5 | 3.36 | 14.5 | 13 | 27.6 | 30.5 |

FIG. 8 is a graph showing cell binding activity of the chimeric antibodies according to an embodiment in CLL1 negative cells and various CLL1-expressing cancer cells.

As shown in Table 15 and FIG. 8, it was confirmed that all the chimeric antibodies according to an embodiment have binding potency in various CLL1-expressing cell lines.

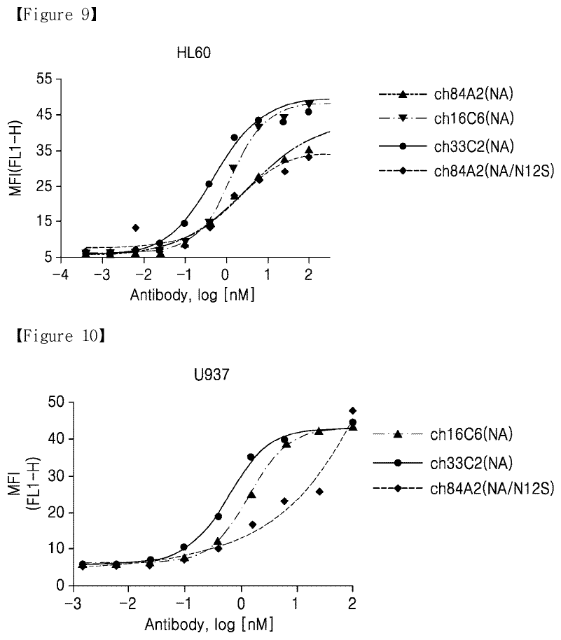

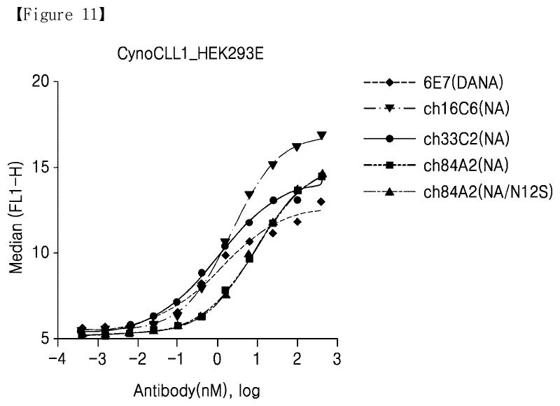

Continuously, the cell binding property of three chimeric antibodies of Example 1.2. are evaluated in HL60 and U937, which are CLL1-expressing tumor cell lines, by using FACS in the same manner as described above. The results are shown in FIGS. 9 and 10.

FIG. 9 is a graph showing cell binding activity of the chimeric antibodies according to an embodiment in HL60.

FIG. 10 is a graph showing cell binding activity of the chimeric antibodies according to an embodiment in U937.

As shown in FIGS. 9 and 10, it was confirmed that all the chimeric antibodies according to an embodiment have binding potency in leukemic cell line, and especially, ch16C6 and ch33C2 clones showed superior binding potency.

Continuously, the cell binding property of three chimeric antibodies of Example 1.2. are evaluated in CynoCLL1_HEK293E by using FACS as a same manner described above. Genentech's anti-CLL-1 antibody 6E7 was used as a reference. The results are shown in FIG. 11.

FIG. 11 is a graph showing cell binding activity of the chimeric antibodies according to an embodiment in HEK293E overexpressing cynomolgus CLL-1.

As shown in FIG. 11, it was confirmed that all the chimeric antibodies according to an embodiment have higher binding potency than 6E7 in HEK293E overexpressing cynomolgus CLL-1. Also, it was confirmed that all three chimeric antibodies according to an embodiment has a higher cell binding activity than that of Genentech's antibody, 6E7.

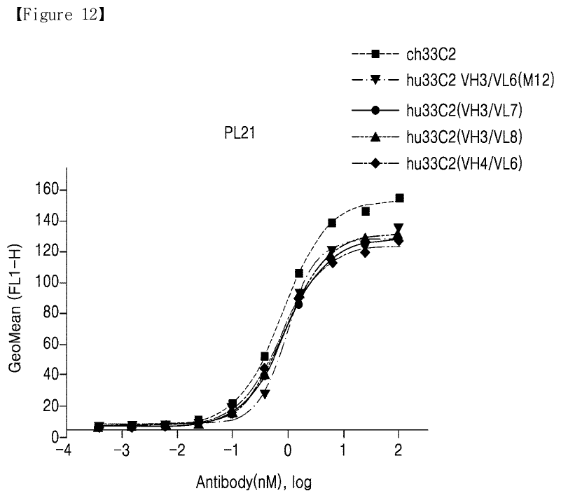

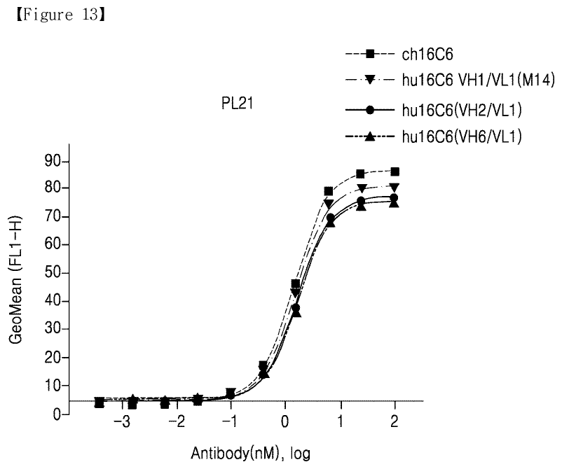

To compare antigen binding properties of the chimeric antibody of Example 1.2. and the humanized candidates of Example 1.3., the antibodies were analyzed using PL21 cell line as a same manner described above. The results are shown in FIGS. 12 and 13.

FIG. 12 is a graph showing cell binding activity of the chimeric antibody and various humanized antibodies hu33C2 according to an embodiment.

FIG. 13 is a graph showing cell binding activity of the chimeric antibody and various humanized antibodies hu16C6 according to an embodiment.

As shown in FIGS. 12 and 13, the humanized antibodies according to an embodiment exhibited cell binding activity equivalent to that of the chimeric antibody according to an embodiment.

### 2.3. Antibody-Dependent Cell Cytotoxicity (ADCC) Evaluation

To assess the in vitro activity of anti-CLL-1 antibodies according to an embodiment in tumor cell killing through effector-mediated immunity, an ADCC reporter bioassay kit (Promega, G7102) was used.

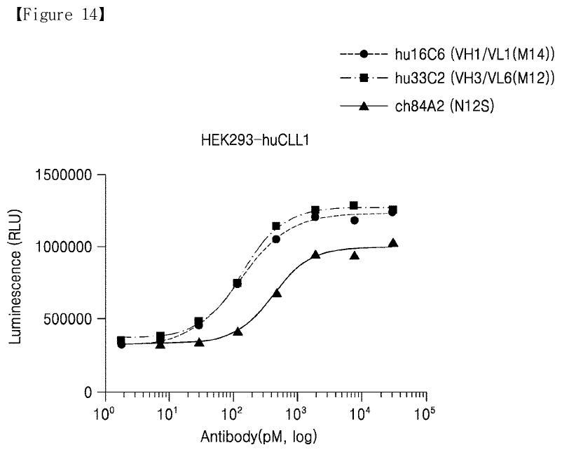

Briefly, Target cells (HEK293-huCLL-1, HEK293, which are cell lines expressing exogenous human CLL-1) were plated into a 96-well flat white bottom plate on the day of the experiment in an assay buffer (RPMI 1640 with 0.5% low IgG FBS). Serial dilutions of antibodies were then added to the plate. The ADCC effector cells were added to each well, and the plates were incubated in a C02 incubator for 6 hrs at 37° C. After the incubation, the plates were left at room temperature for about 10 minutes, and then a Bio-Glo luciferase assay reagent (Promega, G7940) was added to each well. The degree of luminescence was measured with PHERAster FS BMG LABTECH to analyze the degree of ADCC induction. The dose-response curve was fitted with a 4-parameter model using GraphPad Prism 8. The results are shown in FIG. 14.

FIG. 14 is a graph showing ADCC of ch84A2, hu16C6 and hu33C2 according to an embodiment.

As shown in FIG. 14, it was confirmed that the antibodies according to an embodiment, hu16C6 (VH1/VL1(M14)), hu33C2 (VH3/VL6 (M12)), ch84A2 (N12S) induce ADCC by effector cells. And the EC50 of hu16C6 (VH1/VL1(M14)), hu33C2 (VH3/VL6 (M12)), ch84A2 (N12S) were 130.1 pM, 140.5 pM, and 416.5 pM, respectively.

### 2.4. Evaluation of Cytotoxic Activity of ADC (Antibody Drug Conjugation)

To evaluate cytotoxicity of ADC comprising the chimeric antibodies of Example 1.2., various ADCs were prepared using the chimeric antibodies, and cell proliferation inhibition activity thereof was analyzed.

Briefly, Valine (V) at position 205 (according to Kabat numbering, which is also applies below) of the existing antibody light chain is mutated to cysteine (C) and allowed to react with a reducing agent such as dithiothreitol (DTT) to generate a thiol group on the antibody light chain V205C (V205C T), and the antibody was conjugated with a drug by the thioether bond generated between the thiol group and the drug. Hereinafter, three ADSs prepared are referred to as “ch16C6(V205C)-T-AB009”, “ch33C2(V205C)-T-AB009” “ch84A2(V205C)-T-AB009” for each chimeric antibody. Also, Genentech's anti-CLL-1 antibody 6E7 was used as a reference, and ADC of the 6E7 was prepared in the same manner (hereinafter, referred to as “6E7(N54A/V205C)-T-AB009”).

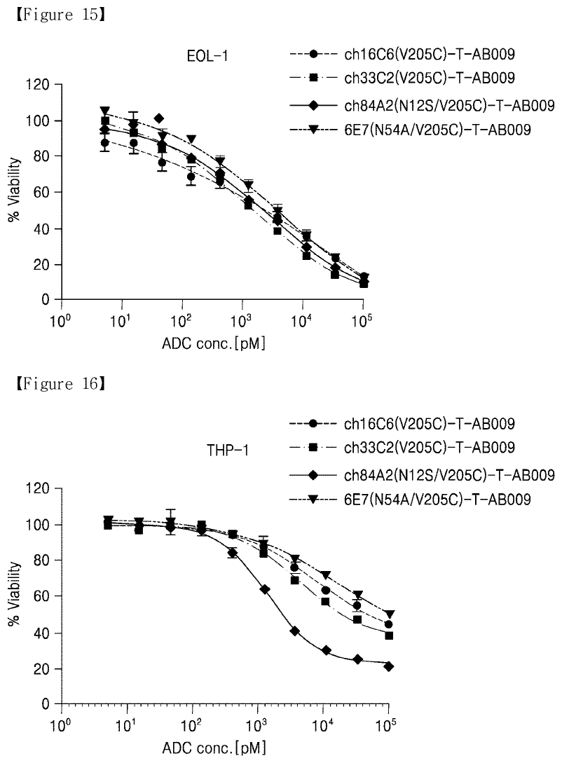

Then, a cancer cell proliferation inhibition activity of the ADCs prepared above was measured using commercially available cancer cell lines (EOL-1, THP-1 cell lines (ATCC)). In a 96-well plate, each well was seeded with 5000 cells of each cancer cell line. After culturing for 24 hours, they were treated with the ADCs of a concentration of 5 to 100000 pM (serially diluted threefold) as shown in Table 16. Six days later, the number of live cells was measured using a WST-8 (Dojindo Molecular Technology Inc.) dye. The results are shown in Table 17, FIGS. 15 and 16.

TABLE 16

| | Final | Stock | | | Total | Final |
|---|---|---|---|---|---|---|
| | Conc. | Conc. | Sample | Media | Volume | Volume |
| | (pM) | (pM) | (μL) | (μL) | (μL) | (μL) |
| 1 | 100000 | 200000 | 3.26 | 446.74 | 450 | 300 |
| 2 | 33333.33 | 66666.67 | 150 | 300 | 450 | 300 |
| 3 | 11111.11 | 22222.22 | 150 | 300 | 450 | 300 |
| 4 | 3703.7 | 7407.41 | 150 | 300 | 450 | 300 |
| 5 | 1234.57 | 2469.13 | 150 | 300 | 450 | 300 |
| 6 | 411.5 | 823.05 | 150 | 300 | 450 | 300 |
| 7 | 137.17 | 274.35 | 150 | 300 | 450 | 300 |
| 8 | 45.72 | 91.45 | 150 | 300 | 450 | 300 |
| 9 | 15.24 | 30.48 | 150 | 300 | 450 | 300 |
| 10 | 5.08 | 10.16 | 150 | 300 | 450 | 450 |

TABLE 17

| | ch16C6(V205C)- | ch33C2(V205C)- | ch84A2(V205C)- | 6E7(N54A/V205C)- |
|---|---|---|---|---|
| | T-AB009 | T-AB009 | T-AB009 | T-AB009 |
| EOL-1 | 18.1 | 1.48 | 2.6 | 3.4 |
| THP-1 | 7.7 | 4.48 | 1.39 | 19.5 |

FIG. 15 is a graph showing cell proliferation inhibition activity of the ADCs according to an embodiment in EOL-1.

FIG. 16 is a graph showing cell proliferation inhibition activity of the ADCs according to an embodiment in THP-1.

As shown in Table 17, FIGS. 15 and 16, it was confirmed that the anti-CLL-1 monoclonal antibodies, 16C6, 33C2 and 84A2 have an improved ability to kill cancer cells than 6E7 in E0L-1 and THP-1 cell lines.

### Example 3. Preparation of Anti-CLL-1/Anti-CD3 Bispecific Antibody

The anti-CLL-1 clone hu33C2 (VH3/VL6 (M12)) prepared in Example 1.3., and CD3 clone disclosed in PCT/CN2018/106618 were selected to prepare an anti-CLL-1/anti-CD3 bispecific antibody. The bispecific antibody was produced according to the WuXiBody generation method disclosed in PCT/CN2018/106766.

Briefly, the DNA fragments of VLA-CL were inserted into a linearized vector which contains a CMV promoter and a human light chain signal peptide. The DNA fragments of VHB-CH1 (33C2)-(G₄S)₃-VHA-CH1 (CD3) or VHB-CH1 (33C2)-(G₄S)₂-VHA-CH1 (CD3) were inserted into a linearized vector containing human IgG1 constant region CH2-CH3 with knob mutations. The DNA fragments of VHB-CH1 were inserted into a linearized vector containing human IgG1 constant region CH2-CH3 with a hole and N297A mutations. Both vectors contained a CMV promoter and a human antibody heavy chain signal peptide. The DNA fragments of VHB-CL were inserted into a linearized vector which contains a CMV promoter and a human light chain signal peptide. Wherein VLA-CL is a light chain variable domain of anti-CD3 antibody (VLA)—a light chain constant domain of anti-CD3 antibody (CL), VHB-CH1 is a heavy chain variable domain of anti-CLL-1 antibody (VHB)—the first constant domain of a heavy chain of anti-CLL-1 antibody (CHi), G₄S is a peptide linker consisting of amino acid sequence of GGGGS, VHA-CH1 is a heavy chain variable domain of anti-CD3 antibody (VHB)—the first constant domain of a heavy chain of anti-CD3 antibody (CHi), and VHB-CL is a heavy chain variable domain of anti-CLL-1 antibody (VHB)—a light chain constant domain of anti-CLL-1 antibody (CL).

The DNA ratio for expression of light chain of anti-CD3 part: heavy chain of anti-CD3 part: heavy chain of anti-CLL1 part: light chain of anti-CLL1 part were 4:1:1:2. 2.94×10⁶/mL of Expi293F cells with a viability higher than 95% were prepared in 300 mL of cell culture medium. Plasmid DNA and ExpiFectamine™293 Reagent were mixed and then added to the cell culture medium.

The cell culture was incubated in a platform shaker at a rotation rate of 150 rpm. The temperature was maintained at 37° C. while CO₂ level was maintained at 8%. After six days of incubation, cells were pelleted by centrifuging at 4000 rpm, at 25° C. for 10 minutes. The supernatant was collected for purification and gel electrophoresis. The supernatant was loaded onto SDS-PAGE gel, following the instructions of NuPAGE™ 4% to 12% Bis-Tris Protein Gels (ThermoFisher), PageRuler™ Unstained Protein Ladder (ThermoFisher) were used alongside the antibody samples to determine the molecular weight of the antibody. The remaining supernatant of each variant was used for subsequence purification. A protein A column was pre-packed with 1 mL of MabSelect Sure resin. The column was equilibrated with 0.1 M of Tris at pH 7.0 before being loaded with the cell culture medium. Subsequently, after the loading, the column was washed with 0.1 M of Tris at pH 7.0 and eluted by 0.1 M of citrate at pH 3.5. The eluted solution was then neutralized by adding 0.1 M of Tris at pH 9.0. The samples were then dialyzed in a PBS buffer (Sangon Biotech, B548117-0500). Finally, the antibodies were filtered through 0.2 μm filters, and aseptically divided into 0.2 mL or 0.5 mL of aliquots in 1.5 mL tubes. Antibodies were frozen, stored at −80° C., and shipped by ABL or in vitro assays at WuXi Biologics.

In conclusion, two kinds of bispecific antibodies that differ only in types of the linker ((G4S)2 or (G4S)3) were produced. The bispecific antibody having (G4S)2 linker is marked as R2 and the one having (G4S)3 was marked as R3. The amino acid sequence of the bispecific antibodies is shown in Table 18. Hereinafter, the bispecific antibody having a R2 linker is also referred to as 33C2/CD3-R2, and the one having a R3 linker is also referred to as 33C2/CD3-R3.

TABLE 18

| | | | SEQ |
|---|---|---|---|
| 33C2/CD3-R2 or | | | ID |
| 33C2/CD3-R3 | | Amino acid sequence | NO: |
| Heavy | Heavy | QVQLVQSGAEVKKPGASVKVSCKASGYSFTGYAMNWVRQAPGQRLEWIGL | 45 |
| Chain-1 | chain | INPYNGGAMYNQKFKGRATLTVDTSASTAYMELSSLRSEDTAVYYCARDY | |
| | of CLL1 | RYEGHLDYWGQGTLVTVSSASTKGPSVFPLAPSSKSTSGGTAALGCLVKD | |
| | | YFPEPVTVSWNSGALTSGVHTFPAVLQSSGLYSLSSVVTVPSSSLGTQTY | |
| | | ICNVNHKPSNTKVDKKVEPKSC | |
| | Linker | GGGGSGGGGS | 59 |
| | (G4S)2 | | |
| | for R2 | | |
| | Linker | GGGGSGGGGSGGGGS | 63 |
| | (G4S)3 | | |
| | for R3 | | |
| | Heavy | QVQLVQSGAEVKKPGSSVKVSCKASGFAFTDYYIHWVRQAPGQGLEWMGW | 53 |
| | chain | ISPGNVNTKYNENFKGRVTITADKSTSTAYMELSSLRSEDTAVYYCARDG | |
| | of CD3 | YSLYYFDYWGQGTLVTVLEDLKNVFPPEVAVFEPSEAEISHTQKATLVCL | |
| | | ATGFYPDHVELSWWVNGKEVHSGVCTDPQPLKEQPALQDSRYALSSRLRV | |
| | | SATFWQNPRNHFRCQVQFYGLSENDEWTQDRAKPVTQIVSAEAWGR | |
| | Fc | ASDKTHTCPPCPAPELLGGPSVFLFPPKPKDTLMISRTPEVTCVVVDVSH | 56 |
| | | EDPEVKFNWYVDGVEVHNAKTKPREEQYASTYRVVSVLTVLHQDWLNGKE | |
| | | YKCKVSNKALPAPIEKTISKAKGQPREPQVYTLPPCREEMTKNQVSLWCL | |
| | | VKGFYPSDIAVEWESNGQPENNYKTTPPVLDSDGSFFLYSKLTVDKSRWQ | |
| | | QGNVFSCSVMHEALHNHYTQKSLSLSPGK | |
| Heavy | Heavy | QVQLVQSGAEVKKPGASVKVSCKASGYSFTGYAMNWVRQAPGQRLEWIGL | 45 |
| Chain-2 | chain | INPYNGGAMYNQKFKGRATLTVDTSASTAYMELSSLRSEDTAVYYCARDY | |
| | of CLL1 | RYEGHLDYWGQGTLVTVSSASTKGPSVFPLAPSSKSTSGGTAALGCLVKD | |
| | | YFPEPVTVSWNSGALTSGVHTFPAVLQSSGLYSLSSVVTVPSSSLGTQTY | |
| | | ICNVNHKPSNTKVDKKVEPKSC | |
| | Fc | DKTHTCPPCPAPELLGGPSVFLFPPKPKDTLMISRTPEVTCVVVDVSHED | 57 |
| | | PEVKFNWYVDGVEVHNAKTKPREEQYASTYRVVSVLTVLHQDWLNGKEYK | |
| | | CKVSNKALPAPIEKTISKAKGQPREPQVCTLPPSREEMTKNQVSLSCAVK | |
| | | GFYPSDIAVEWESNGQPENNYKTTPPVLDSDGSFFLVSKLTVDKSRWQQG | |
| | | NVFSCSVMHEALHNHYTQKSLSLSPGK | |
| Light | Light | EIVLTQSPATLSLSPGERATLSCSATSSVSYMHWYQQKPGQAPRRWIYDT | 46 |
| Chain | Chain | SKLASGIPARFSGSGSGTSYTLTISSLEPEDFAVYYCQQWSSDSPTFGQG | |
| | of CLL1 | TKLEIKRTVAAPSVFIFPPSDEQLKSGTASVVCLLNNFYPREAKVQWKVD | |
| | | NALQSGNSQESVTEQDSKDSTYSLSSTLTLSKADYEKHKVYACEVTHQGL | |
| | | SSPVTKSFNRGEC | |
| | Light | DIVMTQSPDSLAVSLGERATINCKSSQSLLNSRTRKNYLAWYQQKPGQPP | 54 |
| | Chain | KLLIYWASTRQSGVPDRFSGSGSGTDFTLTISSLQAEDVAVYYCTQSHTL | |
| | of CD3 | RTFGGGTKVEIKPDIQNPDPAVYQLRDSKSSDKSVCLFTDFDSQTQVSQS | |
| | | KDSDVYITDKCVLDMRSMDFKSNSAVAWSQKSDFACANAFQNSIIPEDTF | |
| | | FPSPESS | |

### Example 4. Evaluation of Activity of Anti-CLL1/Anti-CD3 Bispecific Antibody

### 4.1. Evaluation of Binding Activity in CLL-1-Expressing Cells (FACS)

To evaluate the tumor antigen binding property of the bispecific antibody of Example 3, the binding ability of CLL1 targeting portions of 33C2/CD3-R2 and 33C2/CD3-R3 to CLL1 expressed in mammalian cells were analyzed by using FACS.

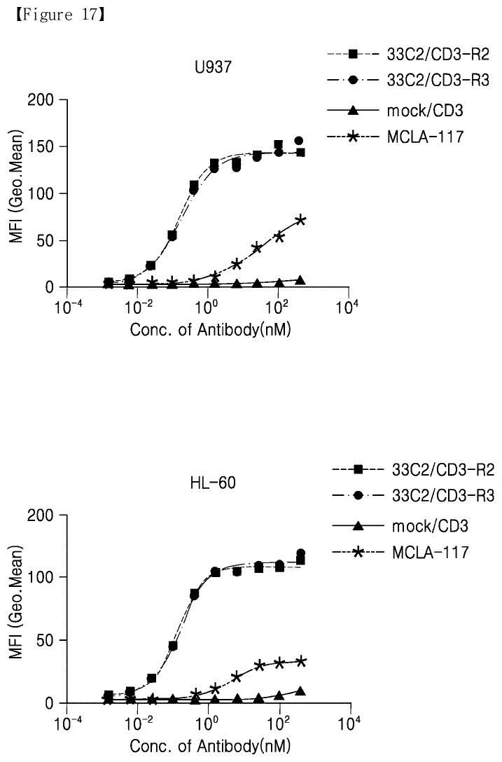

Briefly, CLL1 positive cells (U937 and HL60) were incubated with 33C2/CD3-R2 and 33C2/CD3-R3 antibodies. After washing with a FACS buffer (1% BSA in PBS), the PE-anti-human IgG antibodies were added to each well and incubated at 4° C. for 60 min. The MFI (Median Fluorescence Intensity) of PE (phycoerythrin) was evaluated by FACS Calibur. BsAb mock/CD3, which has an anti-CD3 antigen-binding fragment at one arm, and no antigen-binding fragment at another arm, was used as a control group. Merus's anti-CLL-1/anti-CD3 bispecific antibody MCLA-117 was used as a reference. The results are shown in FIG. 17 and Table 19.

TABLE 19

| | | |
|---|---|---|
| Bispecific antibody | HL-60(EC50) | U937(EC50) |

| | | | |
|---|---|---|---|
| 33C2/CD3-R2 | 0.09 | nM | 0.23 nM |
| 33C2/CD3-R3 | 0.09 | nM | 0.25 nM |
| MCLA-117 | 35.09 | nM | 4.53 nM |

| | | |
|---|---|---|
| mock/CD3 | — | — |

FIG. 17 is a graph showing cell binding activity of the bispecific antibodies according to an embodiment in CLL1 expressing cancer cells.

As shown in FIG. 17 and Table 19, it was confirmed that the binding activity of the bispecific antibodies according to an embodiment in CLL1 positive cancer cell lines was significantly higher than that of MCLA-117 antibodies.

### 4.2. Evaluation of Binding Activity in CD3-Expressing Cells (FACS)

To evaluate the CD3 binding property of the bispecific antibody of Example 3, the binding activity of CD3 targeting portions of 33C2/CD3-R2 and 33C2/CD3-R3 to CD3 expressing mammalian cells were analyzed by using FACS.

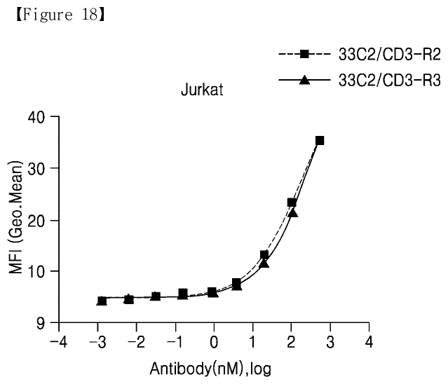

Briefly, CD3 expressing Jurkat cell line was incubated with 33C2/CD3-R2 and 33C2/CD3-R3 antibodies. After washing with a FACS buffer (1% BSA in PBS), the PE-anti-human IgG antibody was added to each well and incubated at 4° C. for 60 min. The MFI (Median Fluorescence Intensity) of PE was evaluated by FACS Calibur. The results are shown in FIG. 18.

FIG. 18 is a graph showing cell binding activity of the bispecific antibodies according to an embodiment.

As shown in FIG. 18, it was confirmed that the bispecific antibodies according to an embodiment strongly bound to CD3 expressing Jurkat cell line in a dose-dependent manner.

### 4.3. In Vitro T Cell Activation of Bispecific Antibodies in Various CLL1-Expressed Conditions

To investigate the CLL1-specific T-cell activation activity of 33C2/CD3-R2 and 33C2/CD3-R3 bispecific antibodies (BsAbs), a Promega kit using AML cell lines (U937 and HL-60) was used.

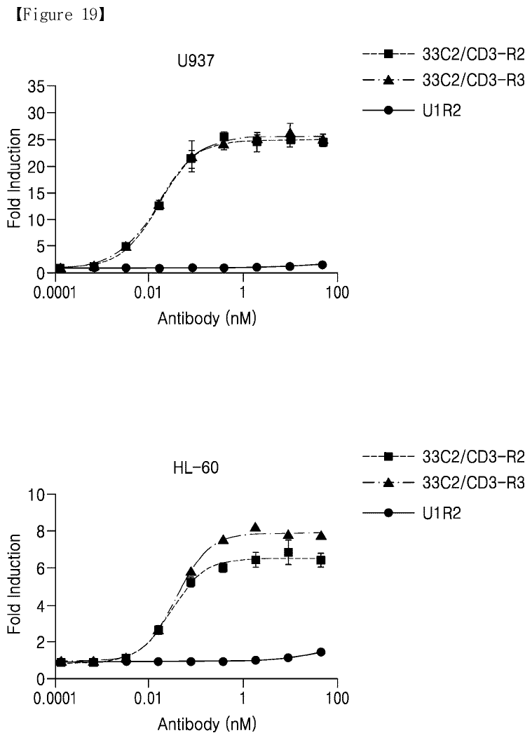

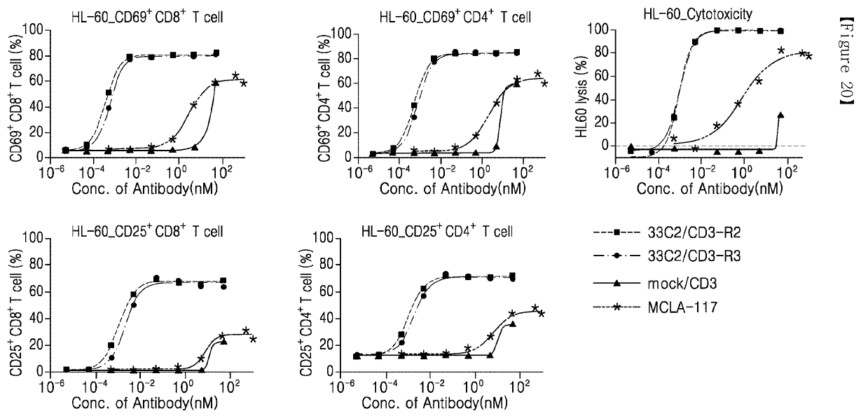

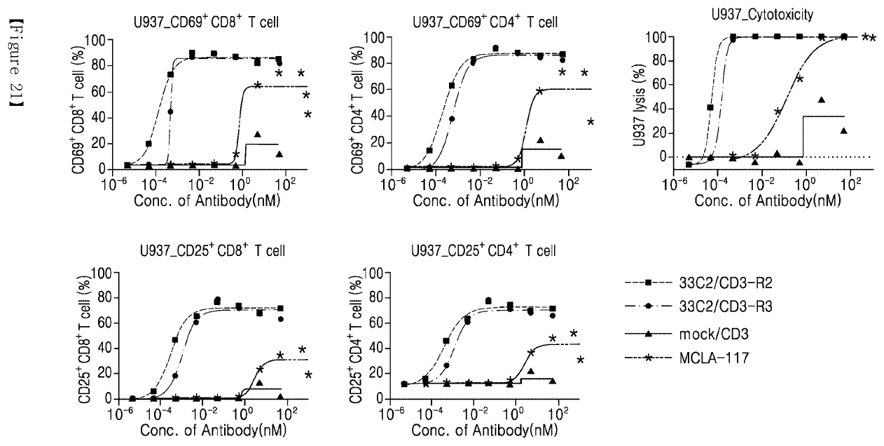

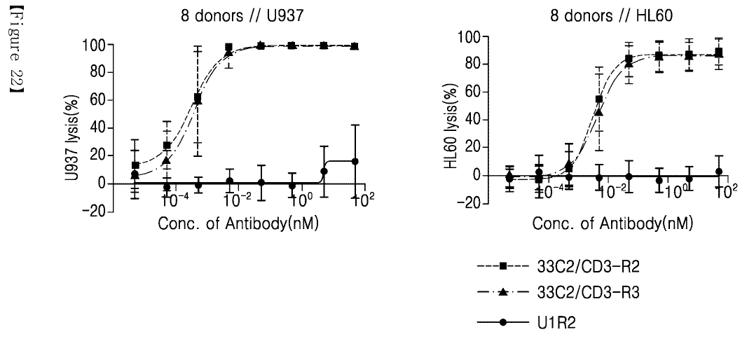

Briefly, 2×10⁴ cells of U937 or HL-60 cancer cells were seeded into 96-well plates in Roswell Park Memorial Institute (RPMI)-1640 medium with 10% FBS. BsAbs and 1×10⁵ TCR/CD3 effector cells (Nuclear factor of activated T-cells, NFAT) were added. T-cell activation was assessed after 6 hours of incubation at 37° C. in 5% CO₂ atmosphere. Then, Bio-Glo Reagent (Promega) was added, and luminescence was measured with a luminescence plate reader. BsAbs U1R2, a bispecific antibody having anti-Claudin18.2 (irrelevant antibody) and anti-CD3 antigen-binding fragments, were used as a negative control. The results are shown in FIG. 19 and Table 20.

TABLE 20

| Bispecific antibody | HL-60(EC50) | U937(EC50) |
|---|---|---|
| 33C2/CD3-R2 | 0.03 nM | 0.016 nM |
| 33C2/CD3-R3 | 0.04 nM | 0.016 nM |
| U1R2 | — | — |

FIG. 19 is a graph showing T cell activation of the bispecific antibodies according to an embodiment.

As shown in FIG. 19 and Table 20, the bispecific antibodies according to an embodiment significantly induced CLL1-specific T-cell activation in a dose-dependent manner, whereas a control group BsAb had no effect.

### 4.4. T Cell Activation and Cytolytic Activity of Bispecific Antibodies in Various CLL1 Expressing AML Cell Lines

To investigate T cell activation and cytolytic activity of the bispecific antibodies of Example 3, FACS was performed in AML cell lines (U937 or HL60).

Briefly, U937 or HL60 suspension target cells (2×10⁴ cells) were seeded onto 96-well U bottom plates. Various concentrations of BsAbs or control molecules (U1R2) and human purified T cells (purified from PBMCs) were added to the plates at an effector:target ratio of 5:1. After 48 hrs, T cell activation and the number of remaining CD33 positive target cells were quantified by flow cytometry. The percentage of specific cell lysis was calculated as follows:

100−[100×(number of target cells treated with BsAbs)/number of target cells number treated with PBS)].

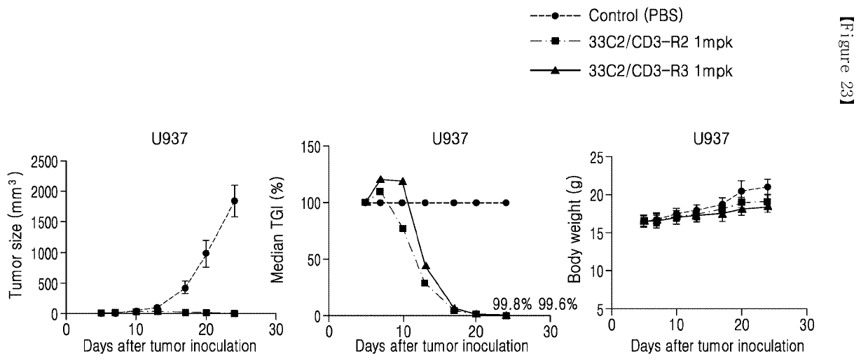

T cells were analyzed by flow cytometry with cell surface levels of CD25 and CD69 as markers of T cell activation. BsAbs mock/CD3 were used as a control group and Merus's anti-CLL-1/anti-CD3 bispecific antibody MCLA-117 was used as a reference. The results of EC50 in HL60 and U937 are shown in FIGS. 20 and 21, respectively, and Tables 21 and 22, respectively.

TABLE 21

| | 33C2/ | 33C2/ | MCLA- | Mock/ |
|---|---|---|---|---|
| Functional | CD3-R2 | CD3-R3 | 117 | CD3 |
| read-out | EC50 (pM) | EC50 (pM) | EC50(pM) | EC50 (pM) |
| CD69 on CD4 T | 0.54 | 0.80 | 2329 | — |
| cells | | | | |
| CD25 on CD4 T | 1.15 | 1.94 | 7116 | — |
| cells | | | | |
| CD69 on CD8 T | 0.36 | 0.57 | 2891 | — |
| cells | | | | |
| CD25 on CD8 T | 1.09 | 2.09 | 7680 | — |
| cells | | | | |
| U937 cell lysis | 0.86 | 0.97 | 799.5 | — |

TABLE 22

| | 33C2/ | 33C2/ | MCLA- | Mock/ |
|---|---|---|---|---|
| Functional | CD3-R2 | CD3-R3 | 117 | CD3 |
| read-out | EC50 (pM) | EC50 (pM) | EC50(pM) | EC50 (pM) |
| CD69 on CD4 T | 0.21 | 0.61 | 1190 | — |
| cells | | | | |
| CD25 on CD4 T | 0.39 | 1.23 | 2526 | — |
| cells | | | | |
| CD69 on CD8 T | 0.14 | ~0.5 | ~713 | — |
| cells | | | | |
| CD25 on CD8 T | 0.30 | 1.10 | 2879 | — |
| cells | | | | |
| U937 cell lysis | 0.05 | 0.15 | 127.7 | — |

FIG. 20 is a graph showing T cell activation and cell lysis activity of the bispecific antibodies according to an embodiment in HL-60.

FIG. 21 is a graph showing T cell activation and cell lysis activity of the bispecific antibodies according to an embodiment in U937.

As shown in FIGS. 20 and 21, and Tables 21 and 22, it was confirmed that the bispecific antibodies according to an embodiment significantly induced CLL1-specific T-cell activation (measured by CD25 and CD69 upregulation) and cancer cell lysis in a dose-dependent manner more than MCLA-117, whereas a control group BsAb had no effect.

### 4.5. In Vitro Cytotoxicity in Various CLL1 Expressing Cell Lines

To determine the ability of the bispecific antibodies according to an embodiment to promote antigen-dependent lysis, in vitro cytotoxicity of BsAbs in FarRed-labelled U937 or HL60 was analyzed.

Briefly, FarRed-labelled U937 or HL6 suspension target cells (1.5×10⁴ cells) were seeded onto 96-well U bottom plates. Various concentrations of BsAbs or control molecules(U1R2) and human PBMC effector cells obtained from various donors were added to the plates at an effector:target ratio of 10:1 or 5:1. All experiments were performed in triplicate. After 48 hrs, target cell killing was assessed using a Zombie Violet™ Fixable Viability Kit (BioLegend, Cat #423114) in flow cytometry. The percentage of specific cell lysis was calculated as follows:

100×[FarRed pos dead cell/(FarRed pos live cell+FarRed pos dead cell)]

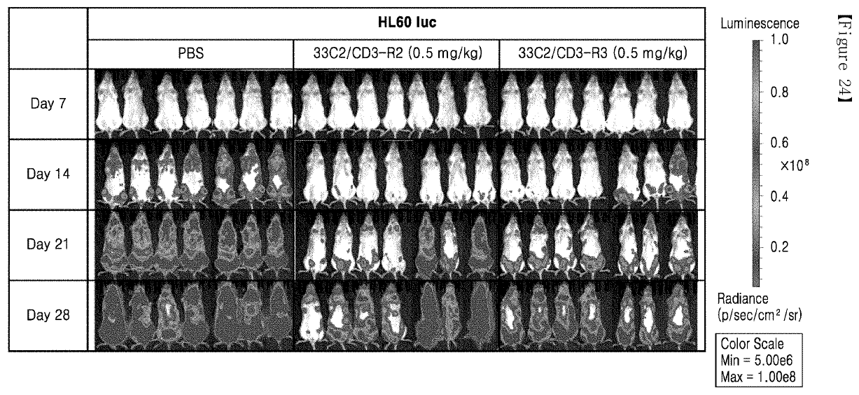

The results are shown in Table 23 and FIG. 22.

TABLE 23

| | | 33C2/CD3-R2 | 33C2/CD3-R3 |
|---|---|---|---|
| Cell line | PBMC donor | (EC50, pM) | (EC50, pM) |
| U937 | ABL19 | 0.32 | 0.39 |
| | ABL16 | 0.05 | 0.03 |
| | ABL15 | 0.16 | 0.04 |
| | ABL06 | 0.54 | 0.57 |
| | ABL17 | 1.1 | 1.69 |
| | ABL10 | 0.05 | 0.15 |
| | ABL18 | 1.63 | 3.05 |
| | ABL09 | 0.13 | 0.08 |
| HL-60 | ABL19 | 9.09 | 8.18 |
| | ABL16 | 5.14 | 7.01 |
| | ABL15 | 3.16 | 3.04 |
| | ABL06 | 4.42 | 5.07 |
| | ABL17 | 5.85 | 16.64 |
| | ABL10 | 0.86 | 0.97 |
| | ABL18 | 3.97 | 9.43 |
| | ABL09 | 1.27 | 1.26 |

FIG. 22 is a graph showing antigen-dependent cell lysis activity of the bispecific antibodies according to an embodiment.

As shown in FIG. 22 and Table 23, it was confirmed that the bispecific antibodies according to an embodiment significantly induced the T-cell-mediated lysis of CLL1-positive U937 and HL60 cells in a concentration-dependent manner, whereas a control group BsAb (U1R2) had no effect.

### 4.6. In Vivo Efficacy in Xenograft Model (U937 Model)

Efficacy of the bispecific antibodies of Example 3 was evaluated in a subcutaneous U937 xenograft model.

Specifically, in vivo antitumor activity was evaluated in NOG mice with U937 subcutaneous xenografts. Brief experimental schemes are as follows:
Tumor cell line: U937
Mouse: NOG group=9
Tumor cell s.c. injection: 5×10⁶ cells/head
Expansion T cell i.p. injection on day 5: 1×10⁷ cells/head, donors (ABL02T) (E:T=2:1)
hIgG1(Fc block) i.p. injection on day 6
Drug (BsAbs) i.p. injection start on day 7(1 mpk, 2QW (twice per week), total 6 times)
Grouping: on day 7;
Group 1(control group): PBS (vehicle);
Group 2: 33C2/CD3-R2; and
Group 3: 33C2/CD3-R3

Tumor size, Median Tumor growth inhibition (% TGI), and Body weight was measured on days 5, 7, 10, 13, 17, 20, and 24.

Since a CD3 arm of the bispecific antibodies has no cross-reactivity with mouse CD3, human T cells were injected intraperitoneally into the mice on day 5, after which 33C2/CD3-R2(Group 2) and 33C2/CD3-R3(Group 3), or PBS (vehicle) (Group 1) was administered twice per week for 3 weeks at a dose of 1 mg/kg. Tumor size (mm³)(=(W)×(L)×(H)×0.5), Median Tumor growth inhibition (% TGI), and Body weight of the mice were measured, and the results are shown in FIG. 23.

FIG. 23 is a graph showing in vivo efficacy of the bispecific antibodies according to an embodiment in U937 xenograft model.

As shown in FIG. 23, it was confirmed that body weights of the mice did not change significantly in both the experimental groups (Group 2 and Group 3) and the control group (Group 1). Also, it was confirmed that the control group had significantly higher tumor volumes compared to the experimental groups, 33C2/CD3-R2, 33C2/CD3-R3 BsAbs-treated mice. Notably, the bispecific antibodies according to an embodiment induced complete tumor regression in U937 xenograft models.

### 4.7. In Vivo Efficacy in Xenograft Model (HL60-Lu Orthotopic AML Model)

Efficacy evaluation of the bispecific antibodies of Example 3 was conducted in an IV HL60-Lu orthotopic AML model.

Specifically, in vivo antitumor activity was evaluated in NOG mice with HL60-Lu IV (intravenous) xenografts. Brief experimental schemes are as follows:
Tumor cell line: HL 601uc
Mouse: NOG group=9
Tumor cell I.V. injection: 1×10⁷ cells/head
Expansion T cell i.p. injection on day 5: 1.5×10⁷ cells/head, donors (ABL14) (E:T=1.5:1)
hIgG1(Fc block) i.p. injection on day 6, 30 mpk
Drug (BsAbs) i.p. injection start on day 7(2QW (twice per week), total 7 times)
Grouping: on day 7;
Group 1(control group): PBS (vehicle);
Group 2: 33C2/CD3-R2; and
Group 3: 33C2/CD3-R3 BLI (Bioluminescence index) was performed on days 7, 14, 21, and 28.

An evaluation of efficacy of the bispecific antibodies according to an embodiment was conducted in a HL60-Lu orthotopic AML model. Specifically, in the established disseminated HL60-luc model, 33C2/CD3-R2 or 33C2/CD3-R3 treatment was initiated after homing of AML cells to bone marrow was confirmed following an IV injection.

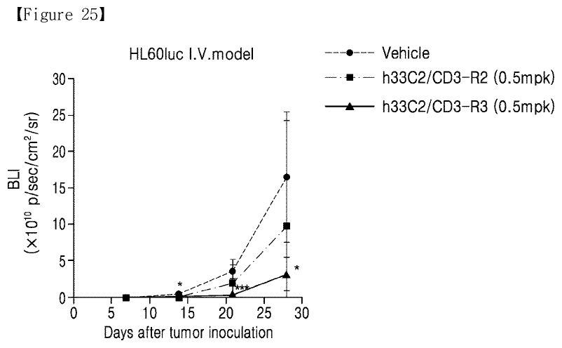

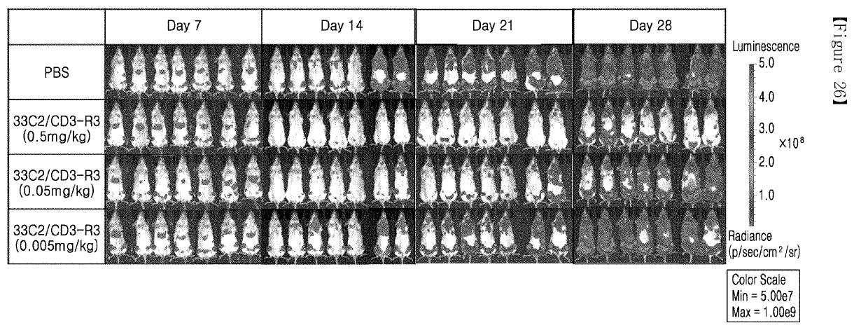

Briefly, to generate a systemic luciferase labeled HL60 orthotopic model, 1×10⁷ HL60-luc cells were injected intravenously in the tail vein on Day 0. Animals were randomized into groups of 7 on Day 6 by bioluminescence intensity. Beginning on Day 7, mice were injected intraperitoneally twice a week 33C2/CD3-R2, 33C2/CD3-R3 at 0.5 mg/kg or vehicle for a total of 7 doses. Analysis of BLI (Bioluminescence index) was performed on days 7, 14, 21, and 28, and the results are shown in FIG. 24 and its quantitative analysis is shown in FIG. 25. Median TGI were also measured on days 14, 21, and 28, and the results are shown in Table 24.

FIG. 24 is images showing BLI (Bioluminescence index) of the administration of the bispecific antibodies according to an embodiment in an HL60-Lu orthotopic AML model.

FIG. 25 is a graph showing results of a quantitative analysis of BLI of the administration of the bispecific antibodies according to an embodiment in HL60-Lu orthotopic AML model (Statistical analysis: Two-way ANOVA (Bonferroni 's multiple comparisons test), *p<0.05, **p<0.01, ***p<0.001.).

TABLE 24

| Median TGI (%) | 33C2/CD3-R2 | 33C2/CD3-R3 |
|---|---|---|
| Day 14 | 94 | 89 |
| Day 21 | 95 | 91 |
| Day 28 | 90 | 80 |

As shown in FIGS. 24 and 25, and Table 24, it was confirmed that the administration of 0.5 mg/kg of the bispecific antibodies according to an embodiment significantly inhibited tumor growth as assessed by bioluminescence (90%, and 80%, respectively) compared with vehicle-treated mice on day 28. It was also confirmed that the administration of the bispecific antibodies according to an embodiment resulted in a significantly reduced tumor burden in the bone marrow, spine, and hind limb as observed by bioluminescence.

### 4.8. Evaluation of Effective Dose in Xenograft Model (HL60-Lu Orthotopic AML Model)

Evaluation of effective dose of 33C2/CD3-R3 bispecific antibodies in HL60-Lu orthotopic AML model was performed in the same manner as in Example 4.7., except that the administered dose of the bispecific antibodies was changed to 0.5 mg/kg, 0.05 mg/kg, and 0.005 mg/kg.

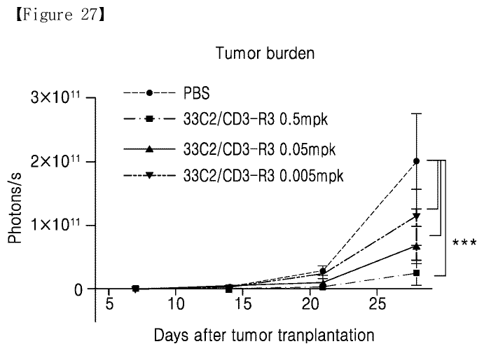

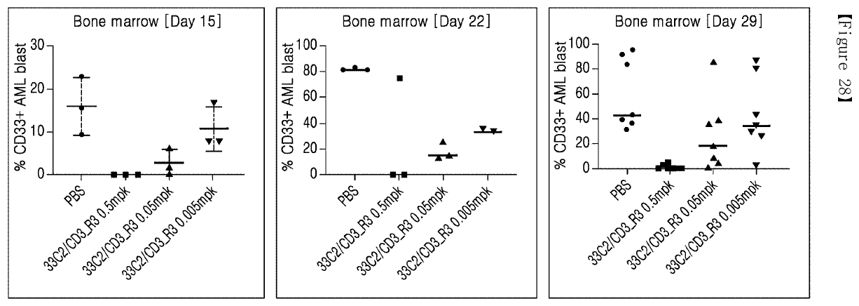

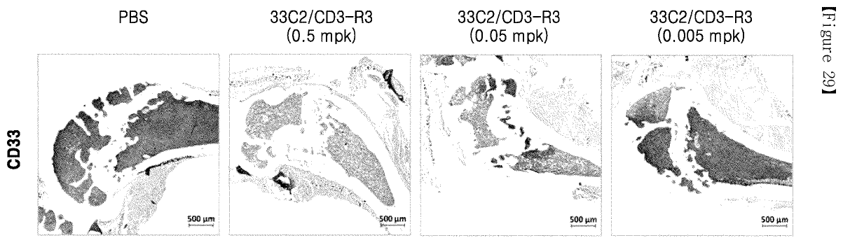

An analysis of BLI (Bioluminescence index) was performed on days 7, 14, 21, and 28, and the results are shown in FIG. 26 and its quantitative analysis is shown in FIG. 27, Median TGIs on days 14, 21, and 28 were also measured, and the results are shown in Table 25. In addition, tumor cells in the bone marrow were measured by using a flow cytometric analysis and IHC staining, and the results are shown in FIGS. 28 and 29, respectively.

FIG. 26 is images showing BLI (Bioluminescence index) of the administration of the bispecific antibodies according to an embodiment in an HL60-Lu orthotopic AML model.

FIG. 27 is a graph showing a quantitative analysis of BLI of the administration of the bispecific antibodies according to an embodiment in an HL60-Lu orthotopic AML model (***=P<0.005).

FIG. 28 is graphs showing results of measuring tumor cells in the bone marrow by FACS after the administration of the bispecific antibodies according to an embodiment.

FIG. 29 is images showing results of measuring tumor cells in the bone marrow by IHC staining after the administration of the bispecific antibodies according to an embodiment.

TABLE 25

| Median TGI (%) | Day 14 | Day 21 | Day 28 |
|---|---|---|---|
| 33C2/CD3-R2: 0.5 mpk | 86 | 91 | 86 |
| 33C2/CD3-R2: 0.05 mpk | 60 | 78 | 60 |
| 33C2/CD3-R2: 0.005 mpk | 13 | 32 | 31 |

As shown in FIGS. 26 and 27, and Table 25, it was confirmed that the administration of 0.5 mg/kg, 0.05 mg/kg, 0.005 mg/kg of the bispecific antibodies according to an embodiment significantly inhibited tumor growth as assessed by bioluminescence (91%, 78%, and 32%, respectively) compared with vehicle-treated mice on day 28. It was also confirmed that the administration of the bispecific antibodies according to an embodiment resulted in a significantly reduced tumor burden in a dose-dependent manner in the bone marrow, spine, and hind limb as observed by bioluminescence.

As shown in FIGS. 28 and 29, it was confirmed that tumor cells in the bone marrow were significantly reduced by the administration of the bispecific antibodies according to an embodiment. These results mean that the antibody according to an embodiment may also inhibit cancer metastasis.

4.9. T Cell Activation and Cytotoxicity in AML Blasts from Primary Patient

To investigate activity of the bispecific antibodies of Example 3, AML blasts from PBMC obtained from primary patient were used. AML blasts correspond to conditions closer to the clinical environment than animal models or cell lines.

First, PBMCs were isolated by density-gradient centrifugation using Ficoll (GE Healthcare), stained with antibodies and analyzed using FACS LSR Fortessa (BD Biosciences) immediately after isolation to investigate the expression of CLL1 and CD33 in the AML blast from 4 primary patients. Data were analyzed with FlowJo (BD Biosciences) and GraphPad Prism ver. 9 (GraphPad Software, Inc.) software. Percent-positive cells and relative MFI were determined relative to each IgG negative control staining of AML blast population. The results of the expression of CLL1 and CD3 in total blood cells and AML blasts were shown in Table 26.

TABLE 26

| | Total blood cells | | | AML blasts | | | |
|---|---|---|---|---|---|---|---|
| AML | % | | | | | CLL1 | CD33 |
| Pt | AML | % | E:T | % | % | (R. | (R. |
| No. | blast | CD3+ | ratio | CLL1 | CD33 | MFI) | MFI) |
| 7 | 61.36 | 7.15 | 1:8.58 | 89.36 | 97.58 | 28.15 | 36.17 |
| 8 | 81.98 | 1.565 | 1:52.38 | 97.04 | 98 | 38.55 | 17.39 |
| 10 | 88.62 | 1.76 | 1:50.35 | 92.93 | 99.33 | 64.83 | 72.70 |
| 11 | 45.15 | 10.37 | 1:4.35 | 80.96 | 85.93 | 53.74 | 30.02 |

In 4 patient AML samples, median CLL1 expression in AML blasts was similar to that of CD33, both in terms of percentage of positive cells and MFI (Mean Fluorescence intensity).

The ability of the bispecific antibodies according to an embodiment to induce cytotoxicity and T cell activation was assessed in an ex vivo cytotoxicity assay using PBMC from 11 AML patients.

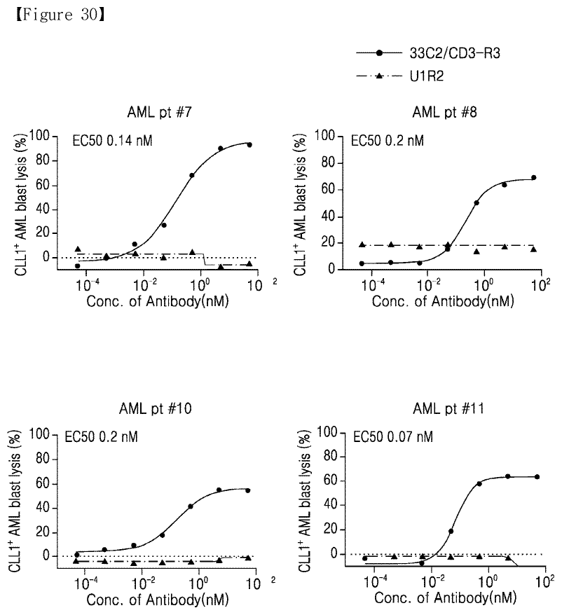

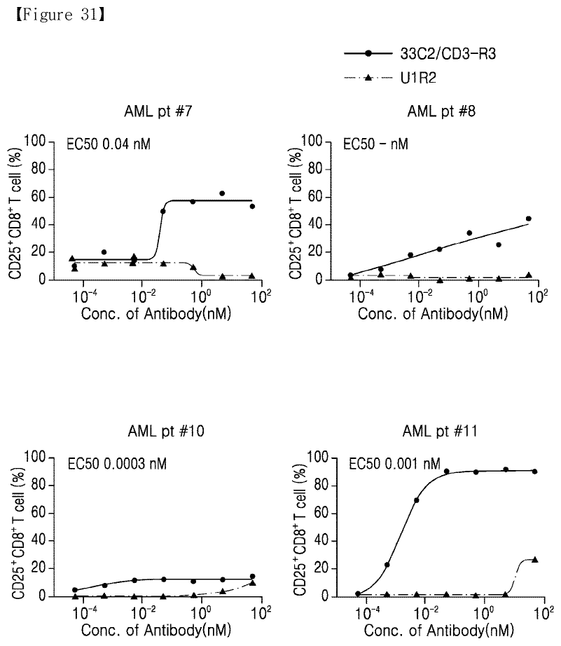

Briefly, AML PBMCs (2×10⁵) isolated from fresh blood from AML patients as above were seeded in triplicate onto 96-well U-bottom plates. BsAbs were added to the plate at 10-fold serial dilutions from 50 nM. U1R2 were used as a control group. After 72 hours, Cell lysis (%) and T cell activation (%) of CLL1 positive AML blasts were analyzed by flow cytometry, and the results are shown in FIGS. 30 and 31, respectively.

FIG. 30 is graphs showing the activity of the cell lysis of the bispecific antibodies according to an embodiment in AML blasts.

FIG. 31 is graphs showing the activity of the T cell activation of the bispecific antibodies according to an embodiment in AML blasts.

As shown in FIG. 30, it was confirmed that the bispecific antibodies according to an embodiment induced a significant cytotoxicity in AML blasts (EC50: 0.07 to 0.2 nM) in a concentration-dependent manner. As shown in FIG. 31, it was confirmed that the results of cytotoxicity as shown above also correlated with increased T-cell activation (EC50: 0.0003 to 0.04 nM) in 4 patient samples. These data indicate that the bispecific antibodies according to an embodiment were effective in killing CLL1 positive AML cells in an ex vivo setting more similar to an in vivo condition.

1. An isolated anti-CLL-1 antibody or an antigen-binding fragment thereof comprising:
(a) a VH CDR1 comprising an amino acid sequence selected from the group consisting of SEQ ID NOs: 1, 2 and 3;
(b) a VH CDR2 comprising an amino acid sequence selected from the group consisting of SEQ ID NOs: 4, 5 and 6;
(c) a VH CDR3 comprising an amino acid sequence selected from the group consisting of SEQ ID NOs: 7, 8, 9, 10 and 11;
(d) a VL CDR1 comprising an amino acid sequence selected from the group consisting of SEQ ID NOs: 12, 13, 14 and 15;
(e) a VL CDR2 comprising an amino acid sequence selected from the group consisting of SEQ ID NOs: 16, 17 and 18; and
(f) a VL CDR3 comprising an amino acid sequence selected from the group consisting of SEQ ID NOs: 19, 20, 21 and 22.

2. The anti-CLL-1 antibody of claim 1,
wherein the anti-CLL-1 antibody or fragment thereof comprises a heavy chain constant region comprising an amino acid sequence selected from the group consisting of SEQ ID NOs: 23 and 24.

3. The anti-CLL-1 antibody of claim 1,
wherein the anti-CLL-1 antibody or fragment thereof comprises a light chain constant region comprising an amino acid sequence consisting of SEQ ID NO: 55.

4. The anti-CLL-1 antibody of claim 1,
wherein the anti-CLL-1 antibody or fragment thereof comprises a heavy chain variable region comprising an amino acid sequence selected from the group consisting of SEQ ID NOs: 25, 26, 27, 28, 29, 30, 31, 32, 33, 34, 35 and 74.

5. The anti-CLL-1 antibody of claim 1,
wherein the anti-CLL-1 antibody or fragment thereof comprises a light chain variable region comprising an amino acid sequence selected from the group consisting of SEQ ID NOs: 36, 37, 38, 39, 40, 41, 42, 43, 44 and 75.

6. The anti-CLL-1 antibody of claim 1,
wherein the anti-CLL-1 antibody or fragment thereof comprises a heavy chain comprising an amino acid sequence consisting of SEQ ID NO: 45.

7. The anti-CLL-1 antibody of claim 14
wherein the anti-CLL-1 antibody or fragment thereof comprises a light chain comprising an amino acid sequence consisting of SEQ ID NO: 46.

8. The anti-CLL-1 antibody of claim 1,
wherein the anti-CLL-1 antibody or fragment thereof comprises the sequence of CDRH1, CDRH2 and CDRH3 of the heavy chain variable region and CDRL1, CDRL2 and CDRL3 of the light chain variable region is any one of the followings:
(a) CDRH1, CDRH2 and CDRH3 are SEQ ID NOs: 1, 4, and 7, respectively, and CDRL1, CDRL2, and CDRL3 are SEQ ID NOs: 12, 16 and 19, respectively;
(b) CDRH1, CDRH2 and CDRH3 are SEQ ID NOs: 2, 5, and 8, respectively, and CDRL1, CDRL2, and CDRL3 are SEQ ID NOs: 13, 17 and 20, respectively;
(c) CDRH1, CDRH2 and CDRH3 are SEQ ID NOs: 3, 6, and 9, respectively, and CDRL1, CDRL2, and CDRL3 are SEQ ID NOs: 14, 18 and 21, respectively;
(d) CDRH1, CDRH2 and CDRH3 are SEQ ID NOs: 1, 4, and 10, respectively, and CDRL1, CDRL2, and CDRL3 are SEQ ID NOs: 15, 16 and 22, respectively; and
(e) CDRH1, CDRH2 and CDRH3 are SEQ ID NOs: 2, 5, and 11, respectively, and CDRL1, CDRL2, and CDRL3 are SEQ ID NOs: 13, 17 and 20, respectively.

9. The anti-CLL-1 antibody of claim 1,
wherein the anti-CLL-1 antibody or antigen-binding fragment thereof is a mouse antibody, a chimeric antibody, a humanized antibody, or a fully human antibody.

10. The anti-CLL-1 antibody of claim 1,
wherein the anti-CLL-1 antibody or antigen-binding fragment thereof is selected from a group consisting of a whole IgG, Fab, Fab′, F(ab′)2, xFab, scFab, dsFv, Fv, scFv, scFv-Fc, scFab-Fc, diabody, minibody, scAb, dAb, half-IgG and combinations thereof.

11. The anti-CLL-1 antibody of claim 1,
which is in the form of IgG1.

12. The anti-CLL-1 antibody of claim 1,
wherein the anti-CLL-1 antibody or antigen-binding fragment thereof is Fab molecule.

13. The anti-CLL-1 antibody of claim 1, wherein the anti-CLL-1 antibody or antigen-binding fragment has one or more of the following characteristics:
(a) binds to human CLL-1;
(b) binds to cynomolgus monkey CLL-1;
(c) binds to CLL-1 on the surface of human peripheral blood mononucleocytes (PBMCs);
(d) binds to CLL-1 on the surface of cynomolgus monkey PBMCs; and
(e) binds to CLL-1 on the surface of a cancer cell.

14. The anti-CLL-1 antibody of claim 1,
a cytotoxic agent is conjugated to at least portion of the anti-CLL-1 antibody or antigen-binding fragment.

15. The anti-CLL-1 antibody of claim 14,
wherein the cytotoxic agent is attached to the anti-CLL-1 antibody or antigen-binding fragment through the linker.

16. The anti-CLL-1 antibody of claim 15,
wherein the linker is cleavable by a protease.

17-18. (canceled)

19. An immunoconjugate comprising a formula Ab-(L-D)p, wherein: (a) Ab is the anti-CLL-1 antibody of claim 1; (b) L is a linker; (c) D is a cytotoxic agent; and (d) p ranges from 1 to 8.

20. An isolated nucleic acid encoding the anti-CLL-1 antibody of claim 1.

21. A method for treating or preventing a cancer in a patient in need thereof, comprising administering to the patient an effective amount of the anti-CLL-1 antibody of claim 1.

22-24. (canceled)

25. The method of claim 21, wherein the cancer is a cancer expressing CLL-1.

26. The method of claim 21,
wherein the cancer is selected from the group consisting of leukemia, rectal cancer, endometrial cancer, nephroblastoma, basal cell carcinoma, nasopharyngeal cancer, bone tumor, esophageal cancer, lymphoma, Hodgkin's lymphoma, non-Hodgkin's lymphoma, follicular thyroid cancer, hepatocellular carcinoma, oral cancer, renal cell carcinoma, multiple myeloma, mesothelioma, osteosarcoma, myelodysplastic syndrome, mesenchymal tumor, soft tissue sarcoma, liposarcoma, gastrointestinal stromal tumor, malignant peripheral nerve sheath tumor (MPNST), ewing sarcoma, leiomyosarcoma, mesenchymal chondrosarcoma, lymphosarcoma, fibrosarcoma, rhabdomyosarcoma, teratoma, neuroblastoma, medulloblastoma, glioma, benign skin tumor, Burkitt's lymphoma, mantle cell lymphoma, diffuse large B cell lymphoma (DLBCL), follicular lymphoma, marginal zone lymphoma, neuroectodermal tumor, epithelial tumor, cutaneous T-cell lymphoma (CTCL), peripheral T cell lymphoma (PTCL), pancreas cancer, haematological malignancies, kidney cancer, tumor vasculature, breast cancer, renal cancer, ovarian cancer, epithelial ovarian cancer, gastric cancer, liver cancer, lung cancer, colorectal cancer, pancreatic cancer, skin cancer, bladder cancer, testicular cancer, uterine cancer, prostate cancer, small cell lung cancer (SCLC), non-small cell lung cancer (NSCLC), neuroblastoma, brain cancer, colon cancer, squamous cell carcinoma, melanoma, myeloma, cervical cancer, thyroid cancer, head and neck cancer and adrenal cancer.

27-29. (canceled)
